SEQUENCE LISTING (45 sequences)

SEQ ID NO: 1 - DNA, 8 bp, Artificial
  Synthetic promoter sequence
cgaccntt

SEQ ID NO: 2 - DNA, 8 bp, Artificial
  Synthetic promoter sequence
ccacgtgc

SEQ ID NO: 3 - DNA, 10 bp, Artificial
  Synthetic promoter sequence
cacgccrcag

SEQ ID NO: 4 - DNA, 10 bp, Artificial
  Synthetic promoter sequence
nanngcsgcw

SEQ ID NO: 5 - DNA, 6 bp, Artificial
  Synthetic promoter sequence
agtcgg

SEQ ID NO: 6 - DNA, 8 bp, Artificial
  Synthetic promoter sequence
tagycrgc

SEQ ID NO: 7 - DNA, 10 bp, Artificial
  Synthetic promoter sequence
trtccgtacg

SEQ ID NO: 8 - DNA, 6 bp, Artificial
  Synthetic promoter sequence
nangag

SEQ ID NO: 9 - DNA, 8 bp, Artificial
  Synthetic promoter sequence
tgatcctt

SEQ ID NO: 10 - DNA, 8 bp, Artificial
  Synthetic promoter sequence
cgaccctt

SEQ ID NO: 11 - DNA, 8 bp, Artificial
  Synthetic promoter sequence
cgaccttt

SEQ ID NO: 12 - DNA, 8 bp, Artificial
  Synthetic promoter sequence
cgtgcctt

SEQ ID NO: 13 - DNA, 8 bp, Artificial
  Synthetic promoter sequence
cgaccatg

SEQ ID NO: 14 - DNA, 8 bp, Artificial
  Synthetic promoter sequence
ccaccgtt

SEQ ID NO: 15 - DNA, 8 bp, Artificial
  Synthetic promoter sequence
cgatcgtt

SEQ ID NO: 16 - DNA, 8 bp, Artificial
  Synthetic promoter sequence
cgacactt

SEQ ID NO: 17 - DNA, 8 bp, Artificial
  Synthetic promoter sequence
cgatcttt

SEQ ID NO: 18 - DNA, 8 bp, Artificial
  Synthetic promoter sequence
cggccctg

SEQ ID NO: 19 - DNA, 8 bp, Artificial
  Synthetic promoter sequence
cgatccgt

SEQ ID NO: 20 - DNA, 8 bp, Artificial
  Synthetic promoter sequence
cgatcctt

SEQ ID NO: 21 - DNA, 10 bp, Artificial
  Synthetic promoter sequence
aaccnancaa

SEQ ID NO: 22 - DNA, 6 bp, Artificial
  Synthetic promoter sequence
acggat

SEQ ID NO: 23 - DNA, 6 bp, Artificial
  Synthetic promoter sequence
acgtac

SEQ ID NO: 24 - DNA, 8 bp, Artificial
  Synthetic promoter sequence
gatggant

SEQ ID NO: 25 - DNA, 12 bp, Artificial
  Synthetic promoter sequence
tcggttcgcgaa

SEQ ID NO: 26 - DNA, 6 bp, Artificial
  Synthetic promoter sequence
tactat

SEQ ID NO: 27 - DNA, 8 bp, Artificial
  Synthetic promoter sequence
aacaggcg

SEQ ID NO: 28 - DNA, 10 bp, Artificial
  Synthetic promoter sequence
cctctctctc

SEQ ID NO: 29 - DNA, 8 bp, Artificial
  Synthetic promoter sequence
gnacgtac

SEQ ID NO: 30 - DNA, 11 bp, Artificial
  Synthetic promoter sequence
ttcaccttcca

SEQ ID NO: 31 - DNA, 9 bp, Artificial
  Synthetic promoter sequence
taccttttc

SEQ ID NO: 32 - DNA, 10 bp, Artificial
  Synthetic promoter sequence
tccttttcct

SEQ ID NO: 33 - DNA, 10 bp, Artificial
  Synthetic promoter sequence
tggtttccct

SEQ ID NO: 34 - DNA, 10 bp, Artificial
  Synthetic promoter sequence
catgcaacaa

SEQ ID NO: 35 - DNA, 8 bp, Artificial
  Synthetic promoter sequence
acaccacg

SEQ ID NO: 36 - DNA, 8 bp, Artificial
  Synthetic promoter sequence
cccccgtt

SEQ ID NO: 37 - DNA, 9 bp, Artificial
  Synthetic promoter sequence
gtttgtttg

SEQ ID NO: 38 - DNA, 10 bp, Artificial
  Synthetic promoter sequence
tngaaacaaa

SEQ ID NO: 39 - DNA, 6 bp, Artificial
  Synthetic promoter sequence
ccacgc

SEQ ID NO: 40 - DNA, 10 bp, Artificial
  Synthetic promoter sequence
tccgtttccc

SEQ ID NO: 41 - DNA, 8 bp, Artificial
  Synthetic promoter sequence
cagactgc

SEQ ID NO: 42 - DNA, 6 bp, Artificial
  Synthetic promoter sequence
ggaacg

SEQ ID NO: 43 - DNA, 6 bp, Artificial
  Synthetic promoter sequence
accatt

SEQ ID NO: 44 - DNA, 8 bp, Artificial
  Synthetic promoter sequence
gsgtgatt

SEQ ID NO: 45 - DNA, 8 bp, Artificial
  Synthetic promoter sequence
gggccnsg

(19) United States
(12) Patent Application Publication
(10) Pub. No.: US 2014/0173779 A1
(43) Pub. Date: Jun. 19, 2014

(54) Methods and Compositions for Effecting Developmental Gene Expression in Plants

(71) Applicants: Syngenta Participations AG, Basel (CH); The University of Guelph, Guelph (CA)

(72) Inventors: Christophe Liseron-Monfils, Guelph (CA); Manish N. Raizada, Guelph (CA); Joseph Colasanti, Guelph (CA); Gregory Downs, Guelph (CA); Yong-Mei Bi, Guelph (CA); Lewis N. Lukens, Guelph (CA); Steven J. Rothstein, Guelph (CA); Tong Zhu, Research Triangle Park, NC (US); Xi Chen, Research Triangle Park, NC (US)

(73) Assignees: The University of Guelph, Guelph (CA); Syngenta Participations AG, Basel (CH)

(21) Appl. No.: 13/857,793

(22) Filed: Apr. 5, 2013

Related U.S. Application Data
(60) Provisional application No. 61/621,016, filed on Apr. 6, 2012.

(51) Int. Cl. C12N 15/82

(52) U.S. Cl. CPC C12N 15/8223
USPC 800/287; 536/24.1; 435/320.1; 435/419; 800/298

(57) ABSTRACT

The invention provides nucleotide sequences that can be used in operable association with a promoter to express a polynucleotide sequence of interest in a plant, plant part or plant cell at particular stages of development and/or in specific tissues. and methods for directing developmental stage specific and/or tissue specific expression of a polynucleotide of interest.

### STATEMENT OF PRIORITY

This application claims the benefit, under 35 U.S.C. §119 (e), of U.S. Provisional Application No. 61/621,016 was filed on Apr. 6, 2012, the entire contents of which is incorporated by reference herein.

### STATEMENT REGARDING ELECTRONIC FILING OF A SEQUENCE LISTING

A Sequence Listing in ASCII text format, submitted under 37 C.F.R. §1.821, entitled 9207-78TS_ST25.txt, 10,169 bytes in size, generated on Apr. 5, 2013 and filed via EFS-Web, is provided in lieu of a paper copy. This Sequence Listing is hereby incorporated herein by reference into the specification for its disclosures.

### FIELD OF THE INVENTION

The present invention relates to compositions and methods for expressing nucleotide sequences in a plant, plant part or plant cell.

### BACKGROUND OF THE INVENTION

Shoot development in higher plants including maize (Zea mays L.) is divided into three phases: juvenile, adult and reproductive (Poethig R S. SCIENCE 1990; 250:923-30; Poethig R S. PLANT PHYSIOLOGY 2010; 154:541-4). Morphological markers can distinguish juvenile from adult leaves: in maize, juvenile leaves are narrow and possess a thin cuticle, but their adaxial surface is covered with epiculticular wax. By contrast, adult maize leaves are wide, have a thick cuticle, and their adaxial surface lacks wax (Poethig R S. SCIENCE 1990; 250:923-30). In maize, the juvenile phase starts with coleoptile leaf emergence (Ve) and ends after emergence of the first 4-6 embryonic leaves which are pre-formed during seed development (Kiesselbach T A. Reproduction and Kernel Development. In: Kiesselbach T A, ed. THE STRUCTURE AND REPRODUCTION OF CORN. New York: Cold Spring Harbor Laboratory press, 1999:63-81; Kiesselbach T A. Development and Structure of Vegetative Parts. In: Kiesselbach T A, ed. THE STRUCTURE AND REPRODUCTION OF CORN. New York: Cold Spring Harbor Laboratory press 1999:8-36). The V1 stage (one leaf-stem node visible) corresponds to two leaves, while the V2 stage (two leaf-stem nodes visible) corresponds to four leaves, which is followed by the adult phase (here, ≧V5 stage, five stem-nodes formed; eight leaves emerged) until all leaf primordia on the main shoot axis are formed. The shoot apical meristem then converts into an inflorescence meristem which initiates the reproductive phase. The reproductive phase is characterized by the emergence of reproductive organs and is further divided into stages of seed development based on the number of days after pollination (DAP). Underground, the juvenile maize root system consists of a single primary root and variable number of seminal roots. Early during juvenile development, by the V2 stage, shoot-borne nodal roots (crown roots) initiate below ground, and subsequently form the backbone of the adult and reproductive root systems (Gaudin et al. PLANT, CELL & ENVIRONMENT 2011; 34:2122-37). Limited information exists about the role that roots play during phase change, though roots have been shown to help regulate the vegetative to reproductive transition in maize (Poethig R S. SCIENCE 1990; 250:923-30).

The existence of these major developmental phases has also been demonstrated at the molecular level. The two phase transitions have been shown to be regulated by transcription factors, miRNAs and chromatin remodelling factors that work together in a complex network that integrates environmental cues (Poethig R S. PLANT PHYSIOLOGY 2010; 154:541-4; Kaufmann et al. Nat Rev Genet 2010; 11:830-42). In maize and Arabidopsis, a small RNA (miRNA156) was found to be a necessary and sufficient regulator of juvenility: it was shown to build up during the juvenile phase then decline during the adult transition (Wu et al. Development 2006; 133:3539-47; Chuck et al. NAT GENET 2007; 39:544-9; Wu et al. CELL 2009; 138:750-9). The targets of miR156 are Squamosa Promoter Binding Protein (SBP) genes (Rhoades et al. CELL 2002; 110:513-20; Schwab et al. DEVELOPMENTAL CELL 2005; 8:517-27). SBP and Squamosa promoter binding-like proteins (SPL) have been shown to be involved in juvenile, adult and reproductive phase transitions (Wu et al. DEVELOPMENT 2006; 133:3539-47; Shikata et al. PLANT AND CELL PHYSIOLOGY 2009; 50:2133-45). When over-expressed, SBP genes targeted by miR156 cause early flowering (Wu et al. Development 2006; 133:3539-47; Cardon et al. THE PLANT JOURNAL 1997; 12:367-77). Recently, the MADS box transcription factor, FLOWERING LOCUS C (FLC), previously shown to repress genes that promote flowering, was also shown to delay the transition from juvenile to adult phases via SPL15 through binding of its cis-element targets, of which 505 were identified in Arabidopsis (Deng et al., PROC. NATL. ACAD. SCI. 108:6680 (2011)). Of these targets, 69% were similar to the canonical MADS box cis-element (CArG motif), while 39% bound to G-A rich cis-acting promoter elements. These observations confirm an antagonistic connection at the molecular level between juvenility and flowering, which has long been known morphologically. The results also demonstrate that large scale transcriptional networks are involved in phase change.

The juvenile phase of maize development, including emergence, is an important stage for seedling establishment. Recent transcriptome studies have described gene expression patterns in juvenile maize leaves, including describing diurnal gene regulation in a juvenile leaf (Jonczyk et al. PLoS ONE 2011; 6:e23628). A remarkable study by Li et al. (Li et al. NAT GENET 2010; 42:1060-7) described gene expression along a tip-base gradient within a juvenile maize leaf, and demonstrated that the growing base of the leaf was dominated by genes involved in respiration, cell elongation and the production of chlorophyll precursors including heme-binding proteins; whereas genes expressed at the tip were involved in photosynthesis, sugar storage and export (Id.). An additional study examined gene expression in maize leaves during the transition from juvenile to adult stages (Strable et al. THE PLANT JOURNAL 2008; 56:1045-57). A final exhaustive study compared gene expression in different organs across maize development, focusing on the lignin biosynthetic pathway (Sekhon et al. THE PLANT JOURNAL 2011; 66:553-63).

### SUMMARY OF THE INVENTION

The present invention provides methods and compositions for expressing polynucleotides of interest in a developmental and/or tissue specific manner in plants, plant parts and plant cells.

Accordingly, in one aspect, the invention provides an isolated nucleic acid comprising a promoter comprising one or more nucleotide sequences selected from the nucleotide sequences of SEQ ID NO:1, SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, and/or SEQ ID NO:45, wherein the promoter confers developmental stage specific transcription when operably linked to a polynucleotide of interest. In some aspects of the invention, a promoter comprising one or more nucleotide sequences of this invention is operably linked to a polynucleotide sequence of interest. In other aspects of the invention, a promoter operably linked to a polynucleotide of interest and comprising one or more nucleotide sequences of this invention can confer juvenile root (e.g., maize Ve-V2) and/or juvenile leaf (e.g., maize Ve-V5) specific transcription; juvenile to adult leaf (e.g., maize Ve to V5) specific transcription; adult to post-flowering leaf specific (e.g., maize V5-R31) transcription; coleoptile specific (e.g., Ve maize) transcription; juvenile leaf specific (e.g., V2 maize) transcription; juvenile root specific (e.g., Ve, V1, V2 maize) transcription; adult leaf specific (e.g., V5, leaf 8, maize) transcription; adult root specific (e.g., V5, maize) transcription; pre-flowering leaf specific (e.g., lower growing) transcription; reproductive stage leaf specific (e.g., fully expanded) transcription; and/or reproductive stage root specific transcription upon the operably linked polynucleotide of interest. As a person of skill in the art would appreciate, a promoter operably linked to a polynucleotide of interest and comprising one or more nucleotide sequences of this invention can be used to effect developmental stage expression and/or tissue specific expression in any angiosperm having analogous developmental stages to those described herein for the nucleotide sequences of this invention. In some aspects, the promoter comprising the nucleotide sequences of this invention can be a minimal promoter.

A further aspect of the present invention provides an isolated nucleic acid comprising a promoter comprising one or more nucleotide sequences of SEQ ID NO:7, SEQ ID NO:27, SEQ ID NO: 28 and/or SEQ ID NO:29, wherein the promoter confers root specific transcription when operably linked to a polynucleotide of interest. In some aspects of the invention, a promoter comprising one or more nucleotide sequences of this invention is operably linked to a polynucleotide sequence of interest. In other aspects of the invention, the root specific transcription is seminal root specific transcription and/or nodal root specific transcription. In some aspects, the promoter is a minimal promoter.

The present invention further provides an expression cassette and/or a vector comprising a nucleic acid of this invention. Additionally, the present invention provides plants, plant parts and/or cells and/or progeny thereof comprising a nucleic acid, an expression cassette, and/or a vector of the present invention.

A further aspect of the invention provides a method for directing juvenile root- and/or juvenile leaf-specific transcription of a polynucleotide of interest in a plant, the method comprising: introducing into a plant cell a nucleic acid comprising a promoter that comprises one or more nucleotide sequences of SEQ ID NO:1, SEQ ID NO:2, SEQ ID NO:3, SEQ ID NO:4, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and/or SEQ ID NO:20, wherein the promoter is operably linked to the polynucleotide of interest; and regenerating a plant from said plant cell, whereby the polynucleotide of interest is expressed in juvenile roots and/or juvenile leaves.

In another aspect of the invention, a method for directing juvenile to adult transition leaf-specific transcription of a polynucleotide of interest in a plant is provided, the method comprising: introducing into a plant cell a nucleic acid comprising a promoter that comprises the nucleotide sequence of SEQ ID NO:5 and/or SEQ ID NO:6, wherein the promoter is operably linked to the polynucleotide of interest; and regenerating a plant from said plant cell, whereby the polynucleotide of interest is expressed in juvenile to adult transition leaves.

A further aspect of the invention provides a method for directing adult to post flowering leaf-specific transcription of a polynucleotide of interest in a plant, the method comprising: introducing into a plant cell a nucleic acid comprising a promoter that comprises the nucleotide sequence of SEQ ID NO:8, wherein the promoter is operably linked to the polynucleotide of interest; and regenerating a plant from said plant cell, whereby the polynucleotide of interest is expressed in adult to post-flowering leaves.

A further aspect of the invention provides a method for directing coleoptile-specific transcription of a polynucleotide of interest in a plant, the method comprising: introducing into a plant cell a nucleic acid comprising a promoter that comprises a nucleotide sequence of SEQ ID NO:21 and/or SEQ ID NO:22, wherein the promoter is operably linked to the polynucleotide of interest; and regenerating a plant from said plant cell, whereby the polynucleotide of interest is expressed in coleoptiles.

A further aspect of the invention provides a method for directing juvenile leaf-specific transcription of a polynucleotide of interest in a plant, the method comprising: introducing into a plant cell a nucleic acid comprising a promoter that comprises one or more nucleotide sequences of SEQ ID NO:23, SEQ ID NO:30, and/or SEQ ID NO:31, wherein the promoter is operably linked to the polynucleotide of interest; and regenerating a plant from said plant cell, whereby the polynucleotide is expressed in juvenile leaves.

A further aspect of the invention provides a method for directing juvenile root-specific transcription of a polynucleotide of interest in a plant, the method comprising: introducing into a plant cell a nucleic acid comprising a promoter that comprises one or more nucleotide sequences of SEQ ID NO:24, SEQ ID NO:25, SEQ ID NO:26, SEQ ID NO:32 and/or SEQ ID NO:33, wherein the promoter is operably linked to the polynucleotide of interest; and regenerating a plant from said plant cell, whereby the polynucleotide of interest is expressed in juvenile roots.

A further aspect of the invention provides a method for directing adult leaf-specific transcription of a polynucleotide of interest in a plant, the method comprising: introducing into a plant cell a nucleic acid comprising a promoter that comprises one or more nucleotide sequences of SEQ ID NO:34, SEQ ID NO:35, and/or SEQ ID NO:36, wherein the promoter is operably linked to the polynucleotide of interest; and regenerating a plant from said plant cell, whereby the polynucleotide of interest is expressed in adult leaves.

A further aspect of the invention provides a method for directing adult root-specific transcription of a polynucleotide of interest in a plant, the method comprising: introducing into a plant cell a nucleic acid comprising a promoter that comprises one or more nucleotide sequences of SEQ ID NO:37, SEQ ID NO:38, SEQ ID NO:39, SEQ ID NO:40, and/or SEQ ID NO:41, wherein the promoter is operably linked to the polynucleotide of interest; and regenerating a plant from said plant cell, whereby the polynucleotide of interest is expressed in adult roots.

A further aspect of the invention provides a method for directing preflowering leaf-specific transcription of a polynucleotide of interest in a plant, the method comprising: introducing into a plant cell a nucleic acid comprising a promoter that comprises the nucleotide sequence of SEQ ID NO:42, wherein the promoter is operably linked to the polynucleotide of interest; and regenerating a plant from said plant cell, whereby the polynucleotide of interest is expressed in pre-flowering leaves.

A further aspect of the invention provides a method for directing reproductive stage leaf-specific transcription of a polynucleotide of interest in a plant, the method comprising: introducing into a plant cell a nucleic acid comprising a promoter that comprises the nucleotide sequence of SEQ ID NO:43, wherein the promoter is operably linked to the polynucleotide of interest; and regenerating a plant from said plant cell, whereby the polynucleotide of interest is expressed in the reproductive stage leaves.

A further aspect of the invention provides a method for directing reproductive stage root-specific transcription of a polynucleotide of interest in a plant, the method comprising: introducing into a plant cell a nucleic acid comprising a promoter that comprises the nucleotide sequence of SEQ ID NO:44 and/or SEQ ID NO:45, wherein the promoter is operably linked to the polynucleotide of interest; and regenerating a plant from said plant cell, whereby the polynucleotide of interest is expressed in the reproductive stage roots.

A further aspect of the invention provides a method for directing root-specific transcription of a polynucleotide of interest in a plant, the method comprising: introducing into a plant cell a nucleic acid comprising a promoter that comprises one or more nucleotide sequences of SEQ ID NO:7, SEQ ID NO:27, SEQ ID NO: 28 and/or SEQ ID NO:29, wherein the promoter is operably linked to the polynucleotide of interest; and regenerating a plant from said plant cell, whereby the polynucleotide of interest is expressed in roots. In some representative embodiments, the promoter comprises the nucleotide sequence of SEQ ID NO:27 and the polynucleotide of interest is expressed in seminal roots. In other embodiments, the promoter comprises the nucleotide sequence of SEQ ID NO:28 and/or SEQ ID NO:29 and the polynucleotide of interest is expressed in nodal roots.

A further aspect of the invention provides a method of producing a plant comprising a nucleic acid of this invention, an expression cassette of this invention and/or a vector of this invention, the method comprising: introducing into a plant cell a nucleic acid of this invention, an expression cassette of this invention and/or a vector of this invention to produce a stably transformed plant cell; and regenerating a stably transformed plant from the plant cell.

The present invention further provides transgenic plants, plants parts including seeds comprising the nucleic acids of this invention, crops comprising said plants, and products produced from the transgenic plants and plant parts of this invention.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### DETAILED DESCRIPTION OF THE INVENTION

Unless the context indicates otherwise, it is specifically intended that the various features of the invention described herein can be used in any combination.

Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

The present invention provides compositions and methods for expressing nucleotide sequences in a plant, plant part or plant cell in a developmental and/or tissue specific manner Specifically, the present inventors have used microarray analysis to identify gene expression clusters in roots and leaves at juvenile, adult, pre-flowering and post-flowering stages and also in roots and leaves at different stages within the juvenile development phase. These gene expression clusters were then used to identify over-represented motifs in the promoters of the genes within each expression cluster. The motifs associated with changes in gene expression from the juvenile-to-adult transition and from pre-flowering to flowering stages are shared by both root and leaf gene clusters. Also identified are shared genes that are up-regulated in both roots and shoots during phase transition. Combined, these results suggest root and shoot coordination of phase change in plant development. Further, the motifs described herein may be used to effect developmentally regulated and/or tissue specific transcription of polynucleotide sequences of interest.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like, is meant to encompass variations of ±20%, ±10%, ±5%, ±1%, ±0.5%, or even ±0.1% of the specified amount.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

Juvenile leaves can be distinguished from adult leaves by certain morphological markers. Thus, for example, for maize, juvenile leaves are narrow and possess a thin cuticle, but their adaxial surface is covered with epicuticular wax. By contrast, adult maize leaves are wide, have a thick cuticle, and their adaxial surface lacks wax.

Further, in maize, the juvenile phase starts with coleoptile leaf emergence (Ve) and ends after emergence of the first 4-6 embryonic leaves which are pre-formed during seed development. The V1 stage (one leaf-stem node visible) corresponds to two leaves, while the V2 stage (two leaf-stem nodes visible) corresponds to four leaves.

The juvenile stage is followed by the adult phase (here, ≧V5 stage, five stem-nodes formed; eight leaves emerged) until all leaf primordia on the main shoot axis are formed. The shoot apical meristem then converts into an inflorescence meristem which initiates the reproductive phase. The reproductive phase is characterized by the emergence of reproductive organs and is further divided into stages of seed development based on the number of days after pollination (DAP).

The juvenile maize root system consists of a single primary root and variable number of seminal roots. In early juvenile development, by the V2 stage, shoot-borne nodal roots (crown roots) initiate below ground, and subsequently form the backbone of the adult and reproductive root systems. In adult and reproductive roots, the main roots are the crown roots from which the lateral roots branch.

As is well-known, all angiosperms have juvenile, adult and reproductive developmental stages and stage transitions therein. Thus, such developmental stages and stage transitions can be observed in, for example, annuals, perennials, monocots, dicots, herbaceous species and tree species all have (Poethig R S. SCIENCE 1990; 250:923-30; Poethig R S. PLANT PHYSIOLOGY 2010; 154:541-4).

These developmental stages have been identified by studying the leaf morphology in, for example, flowering trees including Eucalyptus (See, e.g., Potts, B M and Jordan, G J (1994) “Genetic variation in the juvenile leaf morphology of Eucalyptus globulus Labill. ssp globules,” Forest Genetics, 1 (2):81-95) and in Arabidopsis and maize (See, e.g., Lawson, E. J. & Poethig, R. S. (1995) “Shoot development in plants: time for a change,” Trends Genet. 11:263 268); Kerstetter, R. A. & Poethig, R. S. (1998) “The specification of leaf identity during shoot development,” Anna Rev. Cell Dev. Biol. 14, 373 398). Thus, it is understood that similar developmental stages and stage transitions as described above can be described for various other angiosperms.

The term “modulate” (and grammatical variations) refers to an increase or decrease.

As used herein, the terms “increase,” “increases,” “increased,” “increasing” and similar terms indicate an elevation of at least about 5%, 10%, 15%, 20%, 25%, 30%, 35%, 40%, 45%, 50%, 55%, 60%, 75%, 80%, 85%, 90%, 95%, 100%, 150%, 200%, 300%, 400%, 500% or more as compared to a control (e.g., a plant that does not comprise at least one isolated nucleic acid of the present invention).

As used herein, the terms “reduce,” “reduces,” “reduced,” “reduction” and similar terms mean a decrease of at least about 5%, 10%, 15%, 20%, 25%, 30%, 35%, 40%, 45%, 50%, 55%, 60%, 75%, 80%, 85%, 90%, 95%, or 100% as compared to a control (e.g., a plant that does not comprise at least one isolated nucleic acid of the present invention). In particular embodiments, the reduction results in no or essentially no (i.e., an insignificant amount, e.g., less than about 10%, less than about 5% or even less than about 1%) detectable activity or amount.

As used herein, the term “heterologous” means foreign, exogenous, non-native and/or non-naturally occurring.

As used here, “homologous” means native. For example, a homologous nucleotide sequence or amino acid sequence is a nucleotide sequence or amino acid sequence naturally associated with a host cell into which it is introduced, a homologous promoter sequence is the promoter sequence that is naturally associated with a coding sequence, and the like.

As used herein a “chimeric nucleic acid,” “chimeric nucleotide sequence” or “chimeric polynucleotide” comprises a promoter that is operably linked to one or more nucleotide sequence of this invention (e.g., SEQ ID NOs:1-45) and/or to a polynucleotide of interest each of which are heterologous to the promoter (or vice versa). In particular embodiments, the “chimeric nucleic acid,” “chimeric nucleotide sequence” or “chimeric polynucleotide” comprises a nucleic acid encoding a promoter sequence that is operably linked to one or more nucleotide sequences of this invention and to a heterologous polynucleotide of interest.

A “promoter” is a nucleotide sequence that controls or regulates the transcription of a nucleotide sequence (i.e., a coding sequence) that is operatively associated with the promoter. The coding sequence may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

The nucleotide sequences of the present invention (e.g., promoter motifs, SEQ ID NOs:1-45) can be used in combination with any heterologous promoter nucleotide sequence (e.g., the one or more nucleotide sequences of this invention can be operably associated with a promoter), thereby producing a recombinant or synthetic promoter that confers developmental stage specific transcription when operably linked to a polynucleotide sequence of interest. Thus, in some embodiments, the present invention excludes promoters that are homologous (native) to the nucleotide sequences of the present invention (e.g., SEQ ID NOs:1-45).

A “heterologous promoter” is any promoter that is heterologous (e.g., foreign or non-native) to a nucleotide sequence of the invention (e.g., a promoter motif as described herein).

The choice of promoters useable with the present invention can be made among many different types of promoters. Thus, the choice of promoter depends upon several factors, including, but not limited to, cell- or tissue-specific expression, desired expression level, efficiency, inducibility and/or selectability. For example, where expression in a specific tissue or organ is desired in addition to inducibility, a tissue-specific promoter can be used (e.g., a root specific promoter). In contrast, where expression in response to a stimulus is desired a promoter inducible by other stimuli or chemicals can be used. Where continuous expression is desired throughout the cells of a plant a constitutive promoter can be chosen.

Non-limiting examples of constitutive promoters include cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter.

Some non-limiting examples of tissue-specific promoters useable with the present invention include those encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Thus, in some embodiments, the promoters associated with these tissue-specific nucleic acids can be used in the present invention.

Additional examples of tissue-specific promoters include, but are not limited to, the promoters comprising root hair-specific cis-elements (RHES) (Kim et al. The Plant Cell 18:2958-2970 (2006)), root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al, (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet, 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J 10:2605-2612). Particularly useful for seed-specific expression is the pea vicilin promoter (Czako et al. (1992) Mol. Gen. Genet. 235:33-40; as well as U.S. Pat. No. 5,625,136). Other useful promoters for expression in mature leaves are those that are switched on at the onset of senescence, such as the SAG promoter from Arabidopsis (Gan et al. (1995) Science 270:1986-1988).

In addition, promoters functional in plastids can be used. Non-limiting examples of such promoters include the bacteriophage T3 gene 9 5′ UTR and other promoters disclosed in U.S. Pat. No. 7,579,516. In other embodiments, promoters useful with the present invention include but are not limited to the S-E9 small subunit RuBP carboxylase promoter and the Kunitz trypsin inhibitor gene promoter (Kti3).

In some instances, inducible promoters are useable with the present invention. Examples of inducible promoters useable with the present invention include, but are not limited to, tetracycline repressor system promoters, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters. Other non-limiting examples of inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421) the benzene sulphonamide-inducible promoters (U.S. Pat. No. 5,364,780) and the glutathione S-transferase promoters. Likewise, one can use any appropriate inducible promoter described in Gatz (1996) Current Opinion Biotechnol. 7:168-172 and Gatz (1997) Annu. Rev. Plant Physiol. Plant Mol. Biol, 48:89-108.

In some embodiments of the present invention, a “minimal promoter” or “basal promoter” is used. A minimal promoter is capable of recruiting and binding RNA polymerase II complex and its accessory proteins to permit transcriptional initiation and elongation. In some embodiments, a minimal promoter is constructed to comprise only the nucleotides/nucleotide sequences from a selected promoter that are required for binding of the transcription factors and transcription of a nucleotide sequence of interest that is operably associated with the minimal promoter including but not limited to TATA box sequences. In other embodiments, the minimal promoter lacks cis sequences that recruit and bind transcription factors that modulate (e.g., enhance, repress, confer tissue specificity, confer inducibility or repressibility) transcription. A minimal promoter is generally placed upstream (i.e., 5′) of a nucleotide sequence to be expressed. Thus, nucleotides/nucleotide sequences from any promoter useable with the present invention can be selected for use as a minimal promoter.

Any promoter, such as those described herein, may be altered to generate a minimal promoter by progressively removing nucleotides from the promoter until the promoter ceases to function in order to identify the minimal promoter. Thus, the smallest fragment of a promoter which still functions as a promoter can also be considered a minimal promoter. Accordingly, in some embodiments, a minimal promoter comprising the nucleotide sequences of the invention can be used to drive developmental gene expression in plants. In some particular embodiments, the minimal promoter is a CaMV 35S minimal promoter. Thus, in some embodiments of the invention, a minimal promoter can be operably linked to one or more nucleotide sequences of the invention (e.g., SEQ ID NOs:1-45) and thereby confer developmental stage- or tissue-specific transcription upon a polynucleotide sequence of interest that is also operably linked to said promoter.

Thus, any promoter suitable for use with this invention can be manipulated to produce synthetic or chimeric promoters that combine cis elements from two or more promoters, for example, by adding a heterologous regulatory sequence to an active promoter with its own partial or complete regulatory sequences (Ellis et al., EMBO J. 6:11 16, 1987; Strittmatter and Chua, Proc. Nat. Acad. Sci. USA 84:8986 8990, 1987; Poulsen and Chua, Mol. Gen. Genet. 214:16 23, 1988; Comai et al., Plant. Mol. Biol. 15:373 381, 1991) (See also U.S. Pat. No. 7,202,085). Alternatively, a synthetic promoter can be produced by adding one or more heterologous regulatory sequences (e.g., the nucleotide sequences of this invention, SEQ ID NOs:1-45) to the 5′ upstream region of minimal promoter, (Fluhr et al., Science 232:1106 1112, 1986; Strittmatter and Chua, Proc. Nat. Acad. Sci. USA 84:8986 8990, 1987; Aryan et al., Mol. Gen. Genet. 225:65 71, 1991; Shen and Ho, Physiol. Plantarum 101:653-664 (1997)). Cis elements such as the nucleotide sequences of this invention (SEQ ID NOs:1-45) can be obtained by chemical synthesis or by cloning from promoters that includes such elements, and they can be synthesized with additional flanking sequences that contain useful restriction enzyme sites to facilitate subsequent manipulation.

Thus, in some embodiments, an expression cassette comprising the nucleotide sequences of the present invention (e.g., promoter motifs, SEQ ID NOs:1-45) can comprise other regulatory elements including but not limited to enhancers, introns, targeting sequences, transcriptional regulatory regions, and translational termination regions, insulators, Kozak sequences, 5′ or/and 3′ UTRs, different polyA sequences (terminators), matrix attachment regions (MARS), and the like.

“Polynucleotide of interest” or “nucleotide sequence of interest” refers to any polynucleotide sequence which, when introduced into a plant, confers upon the plant a desired characteristic such as tolerance to abiotic stress, antibiotic resistance, virus resistance, insect resistance, disease resistance, or resistance to other pests, herbicide tolerance, improved nutritional value, improved performance in an industrial process or altered reproductive capability. The “polynucleotide sequence of interest” may also be one that is transferred to plants for the production of commercially valuable products such as enzymes or metabolites in the plant. A “polynucleotide sequence of interest” can encode a polypeptide and/or an inhibitory polynucleotide (e.g., a functional RNA).

A “heterologous polynucleotide sequence of interest” is heterologous (e.g., foreign) to the promoter with which it is operatively associated. Thus, a polynucleotide sequence of interest that is operatively associated with a recombinant or synthetic promoter comprising the polynucleotide sequences of the invention (e.g., SEQ ID NOs:1-45) as described herein is heterologous to that recombinant/synthetic promoter.

A “functional” RNA includes any untranslated RNA that has a biological function in a cell, e.g., regulation of gene expression. Such functional RNAs include but are not limited to RNAi (e.g., siRNA, shRNA), miRNA, antisense RNA, ribozymes, RNA aptamers and the like.

By “operably linked” or “operably associated” as used herein, it is meant that the indicated elements are functionally related to each other, and are also generally physically related. For example, a promoter is operatively linked or operably associated to a coding sequence (e.g., polynucleotide sequence of interest) if it controls the transcription of the sequence. Thus, the term “operatively linked” or “operably associated” as used herein, refers to nucleotide sequences on a single nucleic acid molecule that are functionally associated. Those skilled in the art will appreciate that the control sequences (e.g., promoter) need not be contiguous with the coding sequence, as long as they functions to direct the expression thereof. Thus, for example, intervening untranslated, yet transcribed, sequences can be present between a promoter and a coding sequence, and the promoter sequence can still be considered “operably linked” to the coding sequence.

By the term “express,” “expressing” or “expression” (or other grammatical variants) of a nucleic acid coding sequence, it is meant that the sequence is transcribed. In particular embodiments, the terms “express,” “expressing” or “expression” (or other grammatical variants) can refer to both transcription and translation to produce an encoded polypeptide.

“Wild-type” nucleotide sequence or amino acid sequence refers to a naturally occurring (“native”) or endogenous nucleotide sequence (including a cDNA corresponding thereto) or amino acid sequence.

The terms “nucleic acid,” “polynucleotide” and “nucleotide sequence” are used interchangeably herein unless the context indicates otherwise. These terms encompass both RNA and DNA, including cDNA, genomic DNA, partially or completely synthetic (e.g., chemically synthesized) RNA and DNA, and chimeras of RNA and DNA. The nucleic acid, polynucleotide or nucleotide sequence may be double-stranded or single-stranded, and further may be synthesized using nucleotide analogs or derivatives (e.g., inosine or phosphorothioate nucleotides). Such nucleotides can be used, for example, to prepare nucleic acids, polynucleotides and nucleotide sequences that have altered base-pairing abilities or increased resistance to nucleases. The present invention further provides a nucleic acid, polynucleotide or nucleotide sequence that is the complement (which can be either a full complement or a partial complement) of a nucleic acid, polynucleotide or nucleotide sequence of the invention. Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR §1.822 and established usage.

The nucleic acids and polynucleotides of the invention can be isolated. An “isolated” nucleic acid molecule or polynucleotide is a nucleic acid molecule or polynucleotide that, by the hand of man, exists apart from its native environment and is therefore not a product of nature. An isolated nucleic acid molecule or isolated polynucleotide may exist in a purified form or may exist in a non-native environment such as, for example, a recombinant host cell. Thus, for example, the term “isolated” means that it is separated from the chromosome and/or cell in which it naturally occurs. A nucleic acid or polynucleotide is also isolated if it is separated from the chromosome and/or cell in which it naturally occurs and is then inserted into a genetic context, a chromosome, a chromosome location, and/or a cell in which it does not naturally occur. The recombinant nucleic acid molecules and polynucleotides of the invention can be considered to be “isolated.”

Further, an “isolated” nucleic acid or polynucleotide can be a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. The “isolated” nucleic acid or polynucleotide can exist in a cell (e.g., a plant cell), optionally stably incorporated into the genome. According to this embodiment, the “isolated” nucleic acid or polynucleotide can be foreign to the cell/organism into which it is introduced, or it can be native to an the cell/organism, but exist in a recombinant form (e.g., as a chimeric nucleic acid or polynucleotide) and/or can be an additional copy of an endogenous nucleic acid or polynucleotide. Thus, an “isolated nucleic acid molecule” or “isolated polynucleotide” can also include a nucleotide sequence derived from and inserted into the same natural, original cell type, but which is present in a non-natural state, e.g., present in a different copy number, in a different genetic context and/or under the control of different regulatory sequences than that found in the native state of the nucleic acid molecule or polynucleotide.

In representative embodiments, the “isolated” nucleic acid or polynucleotide is substantially free of cellular material (including naturally associated proteins such as histones, transcription factors, and the like), viral material, and/or culture medium (when produced by recombinant DNA techniques), or chemical precursors or other chemicals (when chemically synthesized). Optionally, in representative embodiments, the isolated nucleic acid or polynucleotide is at least about 10%, 20%, 30%, 40%, 50%, 60%, 70%, 80%, 90%, 95% or more pure.

As used herein, the term “recombinant” nucleic acid, polynucleotide or nucleotide sequence refers to a nucleic acid, polynucleotide or nucleotide sequence that has been constructed, altered, rearranged and/or modified by genetic engineering techniques. The term “recombinant” does not refer to alterations that result from naturally occurring events, such as spontaneous mutations, or from non-spontaneous mutagenesis.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in the cell, i.e., capable of nucleic acid replication under its own control. The term “vector” includes both viral and nonviral (e.g., plasmid) nucleic acid molecules for introducing a nucleic acid into a cell in vitro, ex vivo, and/or in vivo, and is optionally an expression vector. A large number of vectors known in the art may be used to manipulate, deliver and express polynucleotides. Vectors may be engineered to contain sequences encoding selectable markers that provide for the selection of cells that contain the vector and/or have integrated some or all of the nucleic acid of the vector into the cellular genome. Such markers allow identification and/or selection of host cells that incorporate and express the proteins encoded by the marker. A “recombinant” vector refers to a viral or non-viral vector that comprises one or more nucleotide sequences of interest (e.g., transgenes), e.g., two, three, four, five or more polynucleotide sequences of interest.

Viral vectors have been used in a wide variety of gene delivery applications in cells, as well as living animal subjects. Plant viral vectors that can be used include, but are not limited to, geminivirus vectors and/or tobomovirus vectors. Non-viral vectors include, but are not limited to, plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (e.g., delivery to specific tissues, duration of expression, etc.).

The term “fragment,” as applied to a nucleic acid or polynucleotide, will be understood to mean a nucleotide sequence of reduced length relative to the reference or full-length nucleotide sequence and comprising, consisting essentially of and/or consisting of contiguous nucleotides from the reference or full-length nucleotide sequence. Such a fragment according to the invention may be, where appropriate, included in a larger polynucleotide of which it is a constituent. In some embodiments, such fragments can comprise, consist essentially of, and/or consist of oligonucleotides having a length that greater than and/or is at least about 8, 10, 12, 15, 16, 17, 18, 19, 20, 25, 30, 35, 40, 45, 50, 75, 100, 125, 150, 175, 200, 250, 300, 350, 400, 450, 500, 600, 700, 800, 900, 1000 nucleotides (optionally, contiguous nucleotides) or more from the reference or full-length nucleotide sequence, as long as the fragment is shorter than the reference or full-length nucleotide sequence. In representative embodiments, the fragment is a biologically active nucleotide sequence, as that term is described herein.

A “biologically active” nucleotide sequence is one that substantially retains at least one biological activity normally associated with the wild-type nucleotide sequence, for example, promoter activity that confers developmental stage specific and/or tissue specific transcription. In particular embodiments, the “biologically active” nucleotide sequence substantially retains all of the biological activities possessed by the unmodified sequence. By “substantially retains” biological activity, it is meant that the nucleotide sequence retains at least about 50%, 60%, 75%, 85%, 90%, 95%, 97%, 98%, 99%, or more, of the biological activity of the native nucleotide sequence (and can even have a higher level of activity than the native nucleotide sequence). Methods of measuring promoter activity are known in the art.

Two nucleotide sequences are said to be “substantially identical” to each other when they share at least about 60%, 70%, 75%, 80%, 85%, 90%, 95%, 97%, 98%, 99% or even 100% sequence identity. In some particular embodiments, the nucleotide sequences of the present invention include nucleotides sequences having 90%, 95%, 97%, 98%, or 99% sequence identity to the nucleotide sequences of the invention (e.g., SEQ ID NOs:1-45).

Two amino acid sequences are said to be “substantially identical” or “substantially similar” to each other when they share at least about 60%, 70%, 75%, 80%, 85%, 90%, 95%, 97%, 98%, 99% or even 100% sequence identity or similarity, respectively.

As used herein “sequence identity” refers to the extent to which two optimally aligned polynucleotide or polypeptide sequences are invariant throughout a window of alignment of components, e.g., nucleotides or amino acids.

As used herein “sequence similarity” is similar to sequence identity (as described herein), but permits the substitution of conserved amino acids (e.g., amino acids whose side chains have similar structural and/or biochemical properties), which are well-known in the art.

As is known in the art, a number of different programs can be used to identify whether a nucleic acid has sequence identity or an amino acid sequence has sequence identity or similarity to a known sequence. Sequence identity or similarity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35, 351-360 (1987); the method is similar to that described by Higgins & Sharp, CABIOS 5, 151-153 (1989).

Another example of a useful algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. Nucleic Acids Res. 25, 3389-3402 (1997).

The CLUSTAL program can also be used to determine sequence similarity. This algorithm is described by Higgins et al. (1988) Gene 73:237; Higgins et al. (1989) CABIOS 5:151-153; Corpet et al. (1988) Nucleic Acids Res. 16: 10881-90; Huang et al. (1992) CABIOS 8: 155-65; and Pearson et al. (1994) Meth. Mol. Biol. 24: 307-331.

The alignment may include the introduction of gaps in the sequences to be aligned. In addition, for sequences which contain either more or fewer nucleotides than the nucleic acids disclosed herein, it is understood that in one embodiment, the percentage of sequence identity will be determined based on the number of identical nucleotides acids in relation to the total number of nucleotide bases. Thus, for example, sequence identity of sequences shorter than a sequence specifically disclosed herein, will be determined using the number of nucleotide bases in the shorter sequence, in one embodiment. In percent identity calculations relative weight is not assigned to various manifestations of sequence variation, such as, insertions, deletions, substitutions, etc.

Two nucleotide sequences can also be considered to be substantially identical when the two sequences hybridize to each other under stringent conditions. A nonlimiting example of “stringent” hybridization conditions include conditions represented by a wash stringency of 50% formamide with 5×Denhardt's solution, 0.5% SDS and 1×SSPE at 42° C. “Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). In some representative embodiments, two nucleotide sequences considered to be substantially identical hybridize to each other under highly stringent conditions. Generally, highly stringent hybridization and wash conditions are selected to be about 5° C. lower than the thermal melting point (Tₘ) for the specific sequence at a defined ionic strength and pH.

As used herein, the term “polypeptide” encompasses both peptides and proteins (including fusion proteins), unless indicated otherwise.

A “fusion protein” is a polypeptide produced when two heterologous nucleotide sequences or fragments thereof coding for two (or more) different polypeptides not found fused together in nature are fused together in the correct translational reading frame.

An “isolated” polypeptide is a polypeptide that, by the hand of man, exists apart from its native environment and is therefore not a product of nature. An isolated polypeptide may exist in a purified form or may exist in a non-native environment such as, for example, a recombinant host cell.

In representative embodiments, an “isolated” polypeptide means a polypeptide that is separated or substantially free from at least some of the other components of the naturally occurring organism or virus, for example, the cell or viral structural components or other polypeptides or nucleic acids commonly found associated with the polypeptide. In particular embodiments, the “isolated” polypeptide is at least about 1%, 5%, 10%, 25%, 50%, 60%, 70%, 75%, 80%, 85%, 90%, 95%, 97%, 98%, 99% or more pure (w/w). In other embodiments, an “isolated” polypeptide indicates that at least about a 5-fold, 10-fold, 25-fold, 100-fold, 1000-fold, 10,000-fold, or more enrichment of the protein (w/w) is achieved as compared with the starting material.

A “biologically active” polypeptide is one that substantially retains at least one biological activity normally associated with the wild-type polypeptide. In particular embodiments, the “biologically active” polypeptide substantially retains all of the biological activities possessed by the unmodified (e.g., native) sequence. By “substantially retains” biological activity, it is meant that the polypeptide retains at least about 50%, 60%, 75%, 85%, 90%, 95%, 97%, 98%, 99%, or more, of the biological activity of the native polypeptide (and can even have a higher level of activity than the native polypeptide).

“Introducing” in the context of a plant cell, plant tissue, plant part and/or plant means contacting a nucleic acid molecule with the plant cell, plant tissue, plant part, and/or plant in such a manner that the nucleic acid molecule gains access to the interior of the plant cell or a cell of the plant tissue, plant part or plant. Where more than one nucleic acid molecule is to be introduced, these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, e.g., as part of a breeding protocol.

The term “transformation” as used herein refers to the introduction of a heterologous and/or isolated nucleic acid into a cell. Transformation of a cell may be stable or transient. Thus, a transgenic plant cell, plant tissue, plant part and/or plant of the invention can be stably transformed or transiently transformed.

“Transient transformation” in the context of a polynucleotide means that a polynucleotide is introduced into the cell and does not integrate into the genome of the cell.

As used herein, “stably introducing,” “stably introduced,” “stable transformation” or “stably transformed” (and similar terms) in the context of a polynucleotide introduced into a cell, means that the introduced polynucleotide is stably integrated into the genome of the cell (e.g., into a chromosome or as a stable-extra-chromosomal element). As such, the integrated polynucleotide is capable of being inherited by progeny cells and plants.

“Genome” as used herein includes the nuclear and/or plastid genome, and therefore includes integration of a polynucleotide into, for example, the chloroplast genome. Stable transformation as used herein can also refer to a polynucleotide that is maintained extrachromosomally, for example, as a minichromosome.

As used herein, the terms “transformed” and “transgenic” refer to any plant, plant cell, plant tissue (including callus), or plant part that contains all or part of at least one recombinant or isolated nucleic acid, polynucleotide or nucleotide sequence. In representative embodiments, the recombinant or isolated nucleic acid, polynucleotide or nucleotide sequence is stably integrated into the genome of the plant (e.g., into a chromosome or as a stable extra-chromosomal element), so that it is passed on to subsequent generations of the cell or plant.

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, ears, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs plant cell tissue cultures, plant calli, plant clumps, and the like. As used herein, “shoot” refers to the above ground parts including the leaves and stems.

The term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ.

Any plant (or groupings of plants, for example, into a genus or higher order classification) can be employed in practicing the present invention including angiosperms. Thus, a monocot plant and/or a dicot plant can be employed in practicing the present invention. Exemplary plants include, but are not limited to corn (Zea mays), canola (Brassica napus, Brassica rapa ssp.), alfalfa (Medicago saliva), rice (Oryza sativa, including without limitation Indica and/or Japonica varieties), rape (Brassica napus), rye (Secale cereale), sorghum (Sorghum bicolor, Sorghum vulgare), sunflower (Helianthus annus), wheat (Triticum aestivum), soybean (Glycine max), tobacco (Nicotiana tobacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), apple (Maluspumila), blackberry (Rebus), strawberry (Fragaria), walnut (Juglans regia), grape (Vitis vinifera), apricot (Prunus armeniaca), cherry (Prunus), peach (Prunus persica), plum (Prunus domestica), pear (Pyrus communis), watermelon (Citrullus vulgaris), duckweed (Lemna), oats (Avena sativa), barley (Hordium vulgare), vegetables, ornamentals, conifers, and turfgrasses (e.g., for ornamental, recreational or forage purposes), and biomass grasses (e.g., switchgrass and Miscanthus).

Vegetables include Solanaceous species (e.g., tomatoes; Lycopersicon esculentum), lettuce (e.g., Lactuea sativa), carrots (Caucus carota), cauliflower (Brassica oleracea), celery (Apium graveolens), eggplant (Solanum melongena), asparagus (Asparagus officinalis), ochra (Abelmoschus esculentus), green beans (Phaseolus vulgaris), lima beans (Phaseolus limensis), peas (Lathyrus spp.), members of the genus Cucurbita such as Hubbard squash (C. Hubbard), Butternut squash (C. moschata), Zucchini (C. pepo), Crookneck squash (C. crookneck), C. argyrosperma, C. argyrosperma ssp, sororia, C. digitata, C. ecuadorensis, C. foetidissima, C. lundelliana, and C. martinezii, and members of the genus Cucumis such as cucumber (Cucumis sativus), cantaloupe (C. cantalupensis), and musk melon (C. melo).

Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherima), and chrysanthemum.

Conifers, which may be employed in practicing the present invention, include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Turfgrass include but are not limited to zoysia grass, bent grass, fescue grass, bluegrass, St. Augustine grass, Bermuda grass, buffalo grass, rye grass, and orchard grass.

Also included are plants that serve primarily as laboratory models, e.g., Arabidopsis.

### I. Promoter Motif Sequences.

The present invention provides nucleotide sequences (e.g., SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, and/or SEQ ID NO:45) that can be operably associated with a heterologous promoter to produce a recombinant promoter that can confer developmental stage specific transcription, thereby resulting in the expression of a polynucleotide sequence of interest operably linked to said recombinant promoter in roots and/or leaves at specific stages of development (e.g., juvenile (Ve-V2), adult (≧V5), reproductive to post-flowering (V5-R31)). Thus, in some embodiments, the recombinant promoter can confer juvenile root (maize Ve-V2) and juvenile leaf (maize Ve-V5) specific transcription, juvenile to adult leaf (maize Ve to V5) specific transcription, leaf specific (adult to post flowering stages of development, maize V5-R31) transcription, coleoptile specific (Ve maize) transcription, juvenile leaf specific (V2 maize) transcription, juvenile root specific (Ve, V1, V2 maize) transcription, adult leaf specific (V5, leaf 8, maize) transcription, adult root specific (V5, maize) transcription, leaf specific (preflowering, lower growing) transcription, leaf specific (fully expanded reproductive stage) transcription, reproductive stage root specific transcription, seminal root specific transcription and/or nodal root specific transcription on a polynucleotide sequence of interest operably linked to said recombinant promoter.

Accordingly, in additional embodiments, the invention provides a nucleic acid (e.g., a recombinant or isolated nucleic acid) comprising, consisting essentially of, or consisting of a nucleotide sequence selected from the group consisting of: (a) SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, and/or SEQ ID NO:45; (b) a nucleotide sequence that hybridizes to the complete complement of the nucleotide sequence of (a) under stringent hybridization conditions; and (c) a nucleotide sequence having at least about 90%, 95%, 97%, 98%, 99% sequence identity to the nucleotide sequences of any of (a) to (b). In some embodiments, the nucleotide sequence of the present invention is a biologically active promoter motif sequence that can confer developmental stage specific transcription when comprised in a promoter operably associated with a polynucleotide sequence of interest to be expressed, wherein said promoter effects developmental stage transcription and/or tissue specific transcription of the operably associated polynucleotide of interest. In some particular embodiments, the isolated nucleic acid of the present invention does not comprise the nucleotide sequence of SEQ ID NO:1, SEQ ID NO:2, SEQ ID NO:4, SEQ ID NO:5, SEQ ID NO:6, SEQ ID NO:7, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:24, SEQ ID NO:26, SEQ ID NO:42, and/or SEQ ID NO:43. In further embodiments, the isolated nucleic acid of the present invention does not comprise the nucleotide sequence of SEQ ID NO:27, SEQ ID NO:28, SEQ ID NO:29, SEQ ID NO:44, and/or SEQ ID NO:45.

In some embodiments, a recombinant promoter comprising one or more nucleotide sequences of the invention (e.g., SEQ ID NOs:1-45) (e.g., a promoter in operable association with one or more nucleotide sequences of the invention) is operably associated with a polynucleotide of interest. According to this embodiment, a recombinant promoter comprising one or more nucleotide sequences of the invention controls or regulates expression (e.g., transcription and, optionally, translation) of the polynucleotide of interest.

Accordingly, in one aspect, the invention provides an isolated nucleic acid comprising a recombinant promoter comprising, consisting of, or consisting essentially of one or more nucleotide sequences selected from the nucleotide sequences of SEQ ID NO:1, SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, and/or SEQ ID NO:45, wherein said recombinant promoter confers developmental stage specific transcription when operably linked polynucleotide of interest. In representative embodiments, the one or more nucleotide sequences can be a combination of one or more different nucleotide sequences of the invention (e.g., SEQ ID NOs:1-6, 8-26, 30-45), one or more of the same nucleotide sequence of the present invention (e.g., SEQ ID NOs:1-6, 8-26, 30-45), or any combination thereof. In some particular embodiments, the recombinant promoter does not comprise, consist essentially of, or consist of SEQ ID NO:1, SEQ ID NO:2, SEQ ID NO:4, SEQ ID NO:5, SEQ ID NO:6, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:24, SEQ ID NO:26, SEQ ID NO:42, and/or SEQ ID NO:43.

In some aspects of the invention, a promoter comprising one or more nucleotide sequences of this invention is operably linked to a polynucleotide sequence of interest. The promoter comprising the nucleotide sequences of the invention can be any suitable promoter. In some embodiments, the promoter comprising the nucleotide sequences of the invention is a minimal promoter. In particular embodiments, the promoter can be a CaMV 35S minimal promoter.

In other aspects, the invention provides an isolated nucleic acid comprising a recombinant promoter comprising, consisting of, or consisting essentially of one or more nucleotide sequences selected from the nucleotide sequences of SEQ ID SEQ ID NO:2, SEQ ID NO:3, SEQ ID NO:4, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and/or SEQ ID NO:20 that when operably linked to at least one polynucleotide of interest, said promoter confers juvenile root (e.g., maize Ve-V2) and/or juvenile leaf (e.g., maize Ve-V5) specific transcription on said at least one operably linked polynucleotide of interest. In some particular embodiments, the recombinant promoter does not comprise the nucleotide sequence of SEQ ID NO:1, SEQ ID NO:2, SEQ ID NO:4, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and/or SEQ ID NO:20.

In a further aspect, the invention provides an isolated nucleic acid comprising a recombinant promoter comprising, consisting of, or consisting essentially of one or more nucleotide sequences selected from the nucleotide sequences of SEQ ID NO:5 and/or SEQ ID NO:6 that when operably linked to at least one polynucleotide of interest, said promoter confers juvenile to adult transition leaf (e.g., maize Ve to V5) specific transcription on said at least one operably linked polynucleotide of interest. In an additional aspect, the invention provides an isolated nucleic acid comprising a recombinant promoter comprising, consisting of, or consisting essentially of the nucleotide sequence of SEQ ID NO:8 that when operably linked to at least one polynucleotide of interest, said promoter confers adult to post flowering transition leaf specific (e.g., maize V5-R31) transcription on said at least one operably linked polynucleotide of interest. In a further aspect, the invention provides an isolated nucleic acid comprising a recombinant promoter comprising, consisting of, or consisting essentially of one or more nucleotide sequences selected from the nucleotide sequences of SEQ ID NO:21 and/or SEQ ID NO:22 that when operably linked to at least one polynucleotide of interest, said promoter confers coleoptile specific (e.g., Ve maize) transcription on said at least one operably linked polynucleotide of interest.

In other aspects, the invention provides an isolated nucleic acid comprising a recombinant promoter comprising, consisting of, or consisting essentially of one or more nucleotide sequences selected from the nucleotide sequences of SEQ ID NO:23, SEQ ID NO:30, and/or SEQ ID NO:31 that when operably linked to at least one polynucleotide of interest, said promoter confers juvenile leaf specific (e.g., V2 maize) transcription on said at least one operably linked polynucleotide. In further aspects, the invention provides an isolated nucleic acid comprising a recombinant promoter comprising, consisting of, or consisting essentially of one or more nucleotide sequences selected from the nucleotide sequences of SEQ ID NO:34, SEQ ID NO:35, and/or SEQ ID NO:36 that when operably linked to at least one polynucleotide of interest, said promoter confers adult leaf specific (e.g., V5, leaf 8, maize) transcription on said at least one operably linked polynucleotide. Other aspects of the invention provide an isolated nucleic acid comprising a recombinant promoter comprising, consisting of, or consisting essentially of the nucleotide sequence of SEQ ID NO:42 that when operably linked to at least one polynucleotide of interest, said promoter confers pre-flowering leaf specific (lower growing) transcription on said at least one operably linked polynucleotide. In additional aspects, the invention provides an isolated nucleic acid comprising a recombinant promoter comprising, consisting of, or consisting essentially of the nucleotide sequence of SEQ ID NO:43 that when operably linked to at least one polynucleotide of interest, said promoter confers reproductive stage leaf specific (fully expanded) transcription on said at least one operably linked polynucleotide.

Additional aspects of the invention provide an isolated nucleic acid comprising a recombinant promoter comprising, consisting of, or consisting essentially of one or more nucleotide sequences selected from the nucleotide sequences of SEQ ID NO:24 (e.g., Ve), SEQ ID NO:25 (e.g., V1), SEQ ID NO:26 (e.g., V2), SEQ ID NO:32 (e.g., V2) and/or SEQ ID NO:33 (e.g., V2) that when operably linked to at least one polynucleotide of interest, said promoter confers juvenile root specific (e.g., Ve, V1, V2 maize) transcription on said at least one operably linked polynucleotide. Further aspects of the invention provide an isolated nucleic acid comprising a recombinant promoter comprising, consisting of, or consisting essentially of one or more nucleotide sequences selected from the nucleotide sequences of SEQ ID NO:37, SEQ ID NO:38, SEQ ID NO:39, SEQ ID NO:40, and/or SEQ ID NO:41 that when operably linked to at least one polynucleotide of interest, said promoter confers adult root specific (e.g., V5, maize) transcription on said at least one operably linked polynucleotide. In a further aspect, the invention provides an isolated nucleic acid comprising a recombinant promoter comprising, consisting of, or consisting essentially of one or more nucleotide sequences selected from the nucleotide sequences of SEQ ID NO:44 and/or SEQ ID NO:45 that when operably linked to at least one polynucleotide of interest, said promoter confers reproductive stage root specific transcription on said at least one operably linked polynucleotide.

In additional aspects, the invention provides an isolated nucleic acid comprising a recombinant promoter comprising, consisting of, or consisting essentially of one or more nucleotide sequences selected from the nucleotide sequences of SEQ ID NO:7, SEQ ID NO:27, SEQ ID NO: 28 and/or SEQ ID NO:29, wherein the recombinant promoter confers root specific transcription when operably linked to a polynucleotide of interest. In representative embodiments, the one or more nucleotide sequences can be a combination of one or more different nucleotide sequences of the invention (e.g., SEQ ID NOs:7, 27-29), one or more of the same nucleotide sequence of the present invention (e.g., SEQ ID NOs:7, 27-29), or any combination thereof.

In particular aspects, the invention provides an isolated nucleic acid comprising a recombinant promoter comprising, consisting of, or consisting essentially of the nucleotide sequence of SEQ ID NO:27 that when operably linked to at least one polynucleotide of interest, said promoter confers seminal root specific transcription on said at least one operably linked polynucleotide. Still other aspects of the invention provide an isolated nucleic acid comprising a recombinant promoter comprising, consisting of, or consisting essentially of the nucleotide sequence of SEQ ID NO:28 and/or SEQ ID NO:29 that when operably linked to at least one operably linked polynucleotide of interest, said promoter confers nodal root specific transcription on said at least one polynucleotide.

The present invention further provides an expression cassette and/or a vector comprising a nucleic acid of this invention. Additionally, the present invention provides transformed plants, plant parts and/or cells and/or progeny thereof comprising a nucleic acid, an expression cassette, and/or a vector of the present invention.

Thus, the invention also provides an expression cassette comprising a nucleic acid of the invention (e.g., a recombinant promoter comprising a nucleotide sequence of the invention, e.g., SEQ ID NOs:1-45), wherein the recombinant promoter is optionally in operable association with a polynucleotide sequence of interest. The expression cassette can further have a plurality of restriction sites for insertion of a polynucleotide sequence of interest to be operably linked to the regulatory regions. In particular embodiments, the expression cassette comprises more than one (e.g., two, three, four or more) nucleotide sequences of interest.

The expression cassettes of the invention may further comprise a transcriptional termination sequence. Any suitable termination sequence known in the art may be used in accordance with the present invention. The termination region may be native with the transcriptional initiation region, may be native with the polynucleotide sequence of interest, or may be derived from another source. Convenient termination regions are available from the Ti-plasmid of A. tumefaciens, such as the octopine synthetase and nopaline synthetase termination regions. See also, Guerineau et al., Mol. Gen. Genet. 262, 141 (1991); Proudfoot, Cell 64, 671 (1991); Sanfacon et al., Genes Dev. 5, 141 (1991); Mogen et al., Plant Cell 2, 1261 (1990); Munroe et al., Gene 91, 151 (1990); Ballas et al., Nucleic Acids Res. 17, 7891 (1989); and Joshi et al., Nucleic Acids Res. 15, 9627 (1987). Additional exemplary termination sequences are the pea RubP carboxylase small subunit termination sequence and the Cauliflower Mosaic Virus 35S termination sequence. Other suitable termination sequences will be apparent to those skilled in the art.

Further, in particular embodiments, the polynucleotide sequence of interest can be operably associated with a translational start site. The translational start site can be the native translational start site associated with a heterologous polynucleotide sequence of interest, or any other suitable translational start codon.

In illustrative embodiments, the expression cassette includes in the 5′ to 3′ direction of transcription, a promoter comprising, consisting essentially of, consisting of a nucleotide sequence of the present invention (e.g., SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, and/or SEQ ID NO:45), a polynucleotide sequence of interest, and a transcriptional and translational termination region functional in plants.

Those skilled in the art will understand that the expression cassettes of the invention can further comprise enhancer elements and/or tissue preferred elements in combination with the promoter.

Further, in some embodiments, it is advantageous for the expression cassette to comprise a selectable marker gene for the selection of transformed cells. Suitable selectable marker genes include without limitation genes encoding antibiotic resistance, such as those encoding neomycin phosphotransferase II (NEO) and hygromycin phosphotransferase (HPT), as well as genes conferring resistance to herbicidal compounds. Herbicide resistance genes generally code for a modified target protein insensitive to the herbicide or for an enzyme that degrades or detoxifies the herbicide in the plant before it can act. See, DeBlock et al., EMBO J. 6, 2513 (1987); DeBlock et al., Plant Physiol. 91, 691 (1989); Fromm et al., BioTechnology 8, 833 (1990); Gordon-Kamm et al., Plant Cell 2, 603 (1990). For example, resistance to glyphosphate or sulfonylurea herbicides has been obtained using genes coding for the mutant target enzymes, 5-enolpyruvylshikimate-3-phosphate synthase (EPSPS) and acetolactate synthase (ALS). Resistance to glufosinate ammonium, boromoxynil, and 2,4-dichlorophenoxyacetate (2,4-D) have been obtained by using bacterial genes encoding phosphinothricin acetyltransferase, a nitrilase, or a 2,4-dichiorophenoxyacetate monooxygenase, which detoxify the respective herbicides.

Selectable marker genes that can be used according to the present invention further include, but are not limited to, genes encoding: neomycin phosphotransferase II (Fraley et al., CRC Critical Reviews in Plant Science 4, 1 (1986)); cyanamide hydratase (Maier-Greiner et al., Proc. Natl. Acad. Sci. USA 88, 4250 (1991)); aspartate kinase; dihydrodipicolinate synthase (Perl et al., BioTechnology 11, 715 (1993)); the bar gene (Toki et al., Plant Physiol. 100, 1503 (1992); Meagher et al., Crop Sci. 36, 1367 (1996)); tryptophane decarboxylase (Goddijn et al., Plant Mol. Biol. 22, 907 (1993)); neomycin phosphotransferase (NEO; Southern et al., J Mol. Appl. Gen. 1, 327 (1982)); hygromycin phosphotransferase (HPT or HYG; Shimizu et al., Mol. Cell. Biol. 6, 1074 (1986)); dihydrofolate reductase (DHFR; Kwok et al., Proc. Natl. Acad. Sci. USA 83, 4552 (1986)); phosphinothricin acetyltransferase (DeBlock et al., EMBO J. 6, 2513 (1987)); 2,2-dichloropropionic acid dehalogenase (Buchanan-Wollatron et al., J Cell. Biochem. 13D, 330 (1989)); acetohydroxyacid synthase (U.S. Pat. No. 4,761,373 to Anderson et al.; Haughn et al., Mol. Gen, Genet. 221, 266 (1988)); 5-enolpyruvyl-shikimate-phosphate synthase (aroA; Comai et al., Nature 317, 741 (1985)); haloarylnitrilase (WO 87/04181 to Stalker et al.); acetyl-coenzyme A carboxylase (Parker et al., Plant Physiol. 92, 1220 (1990)); dihydropteroate synthase (sulI; Guerineau et al., Plant Mol. Biol. 15, 127 (1990)); and 32 kDa photosystem II polypeptide (psbA; Hirschberg et al., Science 222, 1346 (1983)).

Also included are genes encoding resistance to: chloramphenicol (Herrera-Estrella et al., EMBO J. 2, 987 (1983)); methotrexate (Herrera-Estrella et al., Nature 303, 209 (1983); Meijer et al., Plant Mol. Biol. 16, 807 (1991)); hygromycin (Waldron et al., Plant Mol. Biol. 5, 103 (1985); Zhijian et al., Plant Science 108, 219 (1995); Meijer et al., Plant Mol. Bio. 16, 807 (1991)); streptomycin (Jones et al., Mol. Gen, Genet. 210, 86 (1987)); and spectinomycin (Bretagne-Sagnard et al., Transgenic Res. 5, 131 (1996)); bleomycin (Hille et al., Plant Mol. Biol. 7, 171 (1986)); sulfonamide (Guerineau et al., Plant Mol. Bio. 15, 127 (1990); bromoxynil (Stalker et al., Science 242, 419 (1988)); 2,4-D (Streber et al., Bio/Technology 7, 811 (1989)); phosphinothricin (DeBlock et al., EMBO J 6, 2513 (1987)); spectinomycin (Bretagne-Sagnard and Chupeau, Transgenic Research 5, 131 (1996)).

Other selectable marker genes include the pat gene (for bialaphos and phosphinothricin resistance), the ALS gene for imidazolinone resistance, the HPH or HYG gene for hygromycin resistance, the Hm1 gene for resistance to the Hc-toxin, and other selective agents used routinely and known to one of ordinary skill in the art. See generally, Yarranton, Curr. Opin. Biotech. 3, 506 (1992); Chistopherson et al., Proc. Natl. Acad. Sci. USA 89, 6314 (1992); Yao et al., Cell 71, 63 (1992); Reznikoff, Mol. Microbiol. 6, 2419 (1992); BARKLEY ET AL., THE OPERON 177-220 (1980); Hu et al., Cell 48, 555 (1987); Brown et al., Cell 49, 603 (1987); Figge et al., Cell 52, 713 (1988); Deuschle et al., Proc. Natl. Acad. Sci. USA 86, 5400 (1989); Fuerst et al., Proc. Natl. Acad. Sci. USA 86, 2549 (1989); Deuschle et al., Science 248, 480 (1990); Labow et al., Mol. Cell. Biol. 10, 3343 (1990); Zambretti et al., Proc. Natl. Acad. Sci. USA 89, 3952 (1992); Baim et al., Proc. Natl. Acad. Sci. USA 88, 5072 (1991); Wyborski et al., Nuc. Acids Res. 19, 4647 (1991); Hillenand-Wissman, Topics in Mol. And Struc. Biol. 10, 143 (1989); Degenkolb et al., Antimicrob. Agents Chemother. 35, 1591 (1991); Kleinschnidt et al., Biochemistry 27, 1094 (1988); Gatz et al., Plant J. 2, 397 (1992); Gossen et al., Proc. Natl. Acad. Sci. USA 89, 5547 (1992); Oliva et al., Antimicrob. Agents Chemother. 36, 913 (1992); Hlavka et al., Handbook of Experimental Pharmacology 78 (1985); and Gill et al., Nature 334, 721 (1988).

The polynucleotide sequence of interest can additionally be operably linked to a sequence that encodes a transit peptide that directs expression of an encoded polypeptide of interest to a particular cellular compartment. Transit peptides that target protein accumulation in higher plant cells to the chloroplast, mitochondrion, vacuole, nucleus, and the endoplasmic reticulum (for secretion outside of the cell) are known in the art. Transit peptides that target proteins to the endoplasmic reticulum are desirable for correct processing of secreted proteins. Targeting protein expression to the chloroplast (for example, using the transit peptide from the RubP carboxylase small subunit gene) has been shown to result in the accumulation of very high concentrations of recombinant protein in this organelle. The pea RubP carboxylase small subunit transit peptide sequence has been used to express and target mammalian genes in plants (U.S. Pat. Nos. 5,717,084 and 5,728,925 to Herrera-Estrella et al.). Alternatively, mammalian transit peptides can be used to target recombinant protein expression, for example, to the mitochondrion and endoplasmic reticulum. It has been demonstrated that plant cells recognize mammalian transit peptides that target endoplasmic reticulum (U.S. Pat. Nos. 5,202,422 and 5,639,947 to Hiatt et al.).

Further, the expression cassette can comprise a 5′ leader sequence that acts to enhance expression (transcription, post-transcriptional processing and/or translation) of an operably associated nucleotide sequence of interest. Leader sequences are known in the art and include sequences from: picornavirus leaders, e.g., EMCV leader (Encephalomyocarditis noncoding region; Elroy-Stein et al., Proc. Natl. Acad. Sci USA, 86, 6126 (1989)).; potyvirus leaders, e.g., TEV leader (Tobacco Etch Virus; Allison et al., Virology, 154, 9 (1986)); human immunoglobulin heavy-chain binding protein (BiP; Macajak and Sarnow, Nature 353, 90 (1991)); untranslated leader from the coat protein mRNA of alfalfa mosaic virus (AMV RNA 4; Jobling and Gehrke, Nature 325, 622 (1987)); tobacco mosaic virus leader (TMV; Gallie, Molecular Biology of RNA, 237-56 (1989)); and maize chlorotic mottle virus leader (MCMV; Lommel et al., Virology 81, 382 (1991)). See also, Della-Cioppa et al., Plant Physiology 84, 965 (1987).

### II. Polynucleotide Sequences of Interest.

The polynucleotide sequence(s) of interest in the expression cassette can be any polynucleotide sequence(s) of interest and can be obtained from prokaryotes or eukaryotes (e.g., bacteria, fungi, yeast, viruses, plants, mammals) or the polynucleotide sequence of interest can be synthesized in whole or in part. Further, the polynucleotide sequence of interest can encode a polypeptide of interest or can be transcribed to produce a functional RNA. In particular embodiments, the functional RNA can be expressed to improve an agronomic trait in the plant (e.g., tolerance to drought, heat stress, high temperature, salt, or resistance to herbicides disease, insects or other pests [e.g., a Bacillus thuringiensis endotoxin], and the like), to confer male sterility, to improve fertility and/or enhance nutritional quality (e.g., enzymes that enhance nutritional quality). A polypeptide of interest can be any polypeptide encoded by a polynucleotide sequence of interest. The polynucleotide sequence may further be used in the sense orientation to achieve suppression of endogenous plant genes, as is known by those skilled in the art (see, e.g., U.S. Pat. Nos. 5,283,184; 5,034,323).

The nucleotide sequence of interest can encode a polypeptide that imparts a desirable agronomic trait to the plant (as described above), confers male sterility, improves fertility and/or improves nutritional quality. Other suitable polypeptides include enzymes that can degrade organic pollutants or remove heavy metals. Such plants, and the enzymes that can be isolated therefrom, are useful in methods of environmental protection and remediation. Alternatively, the heterologous nucleotide sequence can encode a therapeutically or pharmaceutically useful polypeptide or an industrial polypeptide (e.g., an industrial enzyme). Therapeutic polypeptides include, but are not limited to antibodies and antibody fragments, cytokines, hormones, growth factors, receptors, enzymes and the like.

Additional non-limiting examples of polypeptides of interest that are suitable for use with this invention (e.g., to be expressed in a developmental stage-specific or tissue specific manner) include polypeptides associated with nutrient uptake including transport and assimilation of organic and inorganic nutrients. Thus, for example, polypeptides involved in nitrogen transport and assimilation, including but not limited to, nitrite transporter (NiTR1 gene), high affinity nitrate transporter, nitrate and chloride transporter, nitrate reductase, NADH-dependent nitrate reductase, oligopeptide and nitrate transporter, ammonium transporter (Osamt1.1; 1.3; 2.2; 3.1; 5.1), nitrate transporter (Atnrtl 1), symbiotic ammonium transporter, ammonium transporter, NADH-dependent glutamate synthase, nitrate transporter, ammonium transporter (Osamt1.1; 5.2), high affinity nitrate transporter (nar2.1), gln4, gl5, nitrate transporter (nrt1.1), amino acid transport protein, NADH-dependent nitrate reductase (nr1, nia1), nitrate transporter (nrt1-5), ammonium transporter (Osamt2.1; 2.3; 3.3), high affinity nitrate transporter (nar2.1; nar2.2), nitrate transporter (Glycine max mt1.2), ferredoxin-dependent glutamate synthase, high affinity nitrate transporter (nrt2.1)

Other non-limiting examples of polypeptides of interest include those involved in resistance to insects, nematodes and pathogenic diseases. Such polypeptides can include but are not limited to glucosinolates (defense against herbivores), chitinases or glucanases and other enzymes which destroy the cell wall of parasites, ribosome-inactivating proteins (RIPs) and other proteins of the plant resistance and stress reaction as are induced when plants are wounded or attacked by microbes, or chemically, by, for example, salicylic acid, jasmonic acid or ethylene, or lysozymes from nonplant sources such as, for example, T4-lysozyme or lysozyme from a variety of mammals, insecticidal proteins such as Bacillus thuringiensis endotoxin, a-amylase inhibitor or protease inhibitors (cowpea trypsin inhibitor), lectins such as wheatgerm agglutinin, RNAses or ribozymes. Further non-limiting examples include nucleic acids which encode the Trichoderma harzianum chit42 endochitinase (GenBank Acc. No.: 578423) or the N-hydroxylating, multi-functional cytochrome P-450 (CYP79) protein from Sorghum bicolor (GenBank Acc. No.: U32624), or functional equivalents of these, chitinases, for example from beans (Brogue et al. (1991) Science 254:1194-1197), “polygalacturonase-inhibiting protein” (PGIP), thaumatine, invertase and antimicrobial peptides such as lactoferrin (Lee T J et al. (2002) J Amer Soc Horticult Sci 127(2):158-164) (See, e.g., U.S. Pat. No. 8,071,749) as well as the plant defense genes, including but not limited to, PR1, BG2, PRS, and NPR1 (or NIM1).

Also useful with the present invention are nucleotide sequences encoding polypeptides involved in plant hormone production or signaling including, but not limited to, auxins, cytokinins, gibberellins, strigolactones, ethylene, jasmonic acid, and brassinosteroids, as well as other nucleotide and polypeptide sequences that regulate or effect root and leaf growth and development. Non-limiting examples of such nucleotide and/or polypeptide sequences include GA-Deficient-1 (GA1; CPS), Gibberellin 20-Oxidase (GA20ox, GA5 (in At)), Gibberellin 2-beta-dioxygenase (GA2ox), Gibberellin 3-Oxidase (GA3ox), GA-Insensitive (GAI), GA Regulated MYB(GAMYB), GCA2 Growth Controlled By ABA 2 (GCA2), G-Protein Coupled Receptor (GCR1), Glycosyl Hydrolase Family-45 (GH45), tryptophan synthase alpha chain (e.g., GRMZM2G046163, GRMZM2G015892), Auxin Binding Protein 1 (ABP1), IAA-amino acid hydrolase ILR1 (e.g., GRMZM2G091540), phosphoribosylanthranilate transferase, Indole Acetic Acid 17/Auxin Resistant 3(IAA17, AXR3), Indole Acetic Acid 3/Short Hypocotyl (IAA3, SHY2), IAA-lysine synthetase (iaaL), tryptophan monooxygenase (iaaM), IAA-Aspartic Acid Hydrolase (IaaspH), IAA-Glucose Synthase (IAGLU), IndoleAcetamide Hydrolase (IAH), Indole-3-Acetaldehyde Oxidase (IAO), IAA-ModifiedProtein (IAP1), Auxin Response factors (ARFs), small auxin up RNA (SAUR), Induced By Cytokinin 6 (Same as ARR5)(IBC6), Induced By Cytokinin 7 (Same as ARR4) IBC7, Viviparous-14 (Vp14), PLA₂ (Zhu J-K. Annual Review of Plant Biology 2002, 53(1):247-273), ATPLC2 (Benschop et al. Plant Physiology 2007, 143(2):1013-1023), inositol polyphosphate 5-phosphatase (At5PTaseI), calcium-dependent protein kinases (CDPKs), calcineurin B-like (CBL) calcium sensor protein CBL4/SOS3, CIPK-like protein 1, ACC (1-aminocyclopropane-1-carboxylate) synthase, ACC oxidase, phosphatase 2C ABI1, TINY, maize lipoxygenase 7 (GRMZM2G070092), allene oxide synthase (AOS) (e.g., GRMZM2G033098 and GRMZM2G376661), short chain alcohol dehydrogenases (ADH), Tasselseed2 (Ts2), Tasselseed1 (Ts1), Supercentipedel (Scn1/GDI1,e.g., AT2G44100), RDH2 (Carol et al. Nature 2005, 438(7070):1013-1016), G-signaling proteins, Morphogenesis of Root Hair (MRH), AtAGC2-1 (e.g., At3g25250), Cellulose Synthase-Like D3 (CSLD3), xylosyltransferase 2 (e.g., At4g02500, AtXX2), xyloglucan endotransglucosylase/hydrolase 26 (e.g., AtXTH26, At4g28850), xyloglucan endotransglycosylase, xyloglucan galact-osyltransferase (MUR3 (e.g., AT2G20370), ARP2/3 (WURM/DISTORTED 1) complex, and germin-like protein (e.g., AT5G39110).

Other nucleotide sequences and polypeptides that are suitable for use with the present invention include those that confer the “stay-green” phenotype (See, Hortensteiner, S. Trends in Plant Science 14: 155-162 (2009)). Non-limiting examples of such nucleotide sequences include MtSGR, MsSGR (Zhou et al. Plant Physiol. 157: 1483-1496 (2011)), STAY-GREEN (SGR or SGN) (Jiang et al., Plant J52: 197-209 (2007)), Park et al., Plant Cell 19: 1649-1664 (2007)), NONYELLOWING (NYE1) (Ren et al., Plant Physiol 144: 1429-1441 (2007)), and/or GREEN-FLESH (GF) or CHLOROPHYLL RETAINER (CL) (Barry et al., Plant Physiol 147: 179-187 (2008)).

Polynucleotides involved in grain filling are also useful with the present invention and include, but are not limited to GIF1 (GRAIN INCOMPLETE FILLING 1) from rice.

Other non-limiting examples of polypeptides of interest that are suitable for production in plants include those resulting in agronomically important traits such as herbicide resistance (also sometimes referred to as “herbicide tolerance”), virus resistance, bacterial pathogen resistance, insect resistance, nematode resistance, and/or fungal resistance. See, e.g., U.S. Pat. Nos. 5,569,823; 5,304,730; 5,495,071; 6,329,504; and 6,337,431. The polypeptide also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, etc.). Various polypeptides of interest, as well as methods for introducing these polypeptides into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Publication No. 2001/0016956. See also, on the World Wide Web at lifesci.sussex.ac.uk/home/Neil_Crickmore/Bt/.

Nucleotide sequences conferring resistance/tolerance to an herbicide that inhibits the growing point or meristem, such as an imidazalinone or a sulfonylurea can also be suitable in some embodiments of the invention. Exemplary nucleotide sequences in this category code for mutant ALS and AHAS enzymes as described, e.g., in U.S. Pat. Nos. 5,767,366 and 5,928,937. U.S. Pat. Nos. 4,761,373 and 5,013,659 are directed to plants resistant to various imidazalinone or sulfonamide herbicides. U.S. Pat. No. 4,975,374 relates to plant cells and plants containing a nucleic acid encoding a mutant glutamine synthetase (GS) resistant to inhibition by herbicides that are known to inhibit GS, e.g., phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

In embodiments of the invention, the nucleotide sequence increases tolerance of a plant, plant part and/or plant cell to heat stress and/or high temperature. The nucleotide sequence can encode a polypeptide or inhibitory polynucleotide (e.g., functional RNA) that results in increased tolerance to heat stress and/or high temperature. Suitable polypeptide include without limitation water stress polypeptides, ABA receptors, and dehydration proteins (e.g., dehydrins (ERDs)).

In representative embodiments, nucleotide sequences that encode polypeptides that provide tolerance to water stress (e.g., drought) are used. Non-limiting examples of polypeptides that provide tolerance to water stress include: water channel proteins involved in the movement of water through membranes; enzymes required for the biosynthesis of various osmoprotectants (e.g., sugars, proline, and Glycine-betaine); proteins that protect macromolecules and membranes (e.g., LEA protein, osmotin, antifreeze protein, chaperone and mRNA binding proteins); proteases for protein turnover (thiol proteases, Clp protease and ubiquitin); and detoxification enzymes (e.g., glutathione S-transferase, soluble epoxide hydrolase, catalase, superoxide dismutase and ascorbate peroxidase). Non-limiting examples of proteins involved in the regulation of signal transduction and gene expression in response to water stress include protein kinases (MAPK, MAPKKK, S6K, CDPK, two-component His kinase, Bacterial-type sensory kinase and SNF1); transcription factors (e.g., MYC and bZIP); phosopholipase C; and 14-3-3 proteins.

Nucleotide sequences that encode receptors/binding proteins for abscisic acid (ABA) are also useful in the practice of the present invention. Non-limiting examples of ABA binding proteins/receptors include: the Mg-chelatase H subunit; RNA-binding protein FCA; G-protein coupled receptor GCR2; PYR1; PYL5; protein phosphatases 2C ABI1 and ABI2; and proteins of the RCAR (Regulatory Component of the ABA Receptor) family.

In embodiments of the invention, the nucleotide sequence encodes a dehydration protein, also known as a dehydrin (e.g., an ERD). Dehyration proteins are a group of proteins known to accumulate in plants in response to dehydration. Examples include WCOR410 from wheat; PCA60 from peach; DHN3 from sessile oak, COR47 from Arabidopsis thaliana; Hsp90, BN59, BN115 and Bnerd10 from Brassica napus; COR39 and WCS19 from Triticum aestivum (bread wheat); and COR25 from Brassica rapa subsp. Pekinensis. Other examples of dehydration proteins are ERD proteins, which include without limitation, ERD1, ERD2, ERD4, ERD5, ERD6, ERD8, ERD10, ERD11, ERD13, ERD15 and ERD16.

Polypeptides encoded by nucleotide sequences conferring resistance to glyphosate are also suitable for use with the present invention. See, e.g., U.S. Pat. No. 4,940,835 and U.S. Pat. No. 4,769,061. U.S. Pat. No. 5,554,798 discloses transgenic glyphosate resistant maize plants, which resistance is conferred by an altered 5-enolpyruvyl-3-phosphoshikimate (EPSP) synthase gene. Heterologous nucleotide sequences suitable to confer tolerance to the herbicide glyphosate also include, but are not limited to the Agrobacterium strain CP4 glyphosate resistant EPSPS gene (aroA:CP4) as described in U.S. Pat. No. 5,633,435 or the glyphosate oxidoreductase gene (GOX) as described in U.S. Pat. No. 5,463,175. Other heterologous nucleotide sequences include genes conferring resistance to herbicides that act to inhibit the action of acetolactate synthase (ALS), in particular the sulfonylurea-type herbicides (e.g., mutant forms of the acetolactate synthase (ALS) gene that lead to such resistance, in particular the S4 and/or Hra mutations), genes coding for resistance to herbicides that act to inhibit the action of glutamine synthase, such as phosphinothricin or basta (e.g., the bar gene). The bar gene encodes resistance to the herbicide basta, the nptll gene encodes resistance to the antibiotics kanamycin and geneticin, and the ALS gene encodes resistance to the herbicide chlorsulfuron.

Nucleotide sequences coding for resistance to phosphono compounds such as glufosinate ammonium or phosphinothricin, and pyridinoxy or phenoxy propionic acids and cyclohexones are also suitable. See, European Patent Application No. 0 242 246. See also, U.S. Pat. Nos. 5,879,903, 5,276,268 and 5,561,236.

Other suitable nucleotide sequences of interest include those coding for resistance to herbicides that inhibit photosynthesis, such as a triazine and a benzonitrile (nitrilase). See, U.S. Pat. No. 4,810,648. Additional suitable nucleotide sequences coding for herbicide resistance include those coding for resistance to 2,2-dichloropropionic acid, sethoxydim, haloxyfop, imidazolinone herbicides, sulfonylurea herbicides, triazolopyrimidine herbicides, s-triazine herbicides and bromoxynil. Also suitable are nucleotide sequences conferring resistance to a protox enzyme, or that provide enhanced resistance to plant diseases; enhanced tolerance of adverse environmental conditions (abiotic stresses) including but not limited to drought, heat stress, high temperature, cold, excessive soil salinity or extreme acidity or alkalinity; and alterations in plant architecture or development, including changes in developmental timing. See, e.g., U.S. Patent Publication No. 2001/0016956 and U.S. Pat. No. 6,084,155.

Insecticidal proteins useful in the invention may be produced in an amount sufficient to control insect pests, i.e., insect controlling amounts. It is recognized that the amount of production of insecticidal protein in a plant useful to control insects may vary depending upon the cultivar, type of insect, environmental factors and the like. Suitable heterologous nucleotide sequences that confer insect tolerance include those which provide resistance to pests such as rootworm, cutworm, European Corn Borer, and the like. Exemplary nucleotide sequences include, but are not limited to, those that encode toxins identified in Bacillus organisms (see, e.g., WO 99/31248; U.S. Pat. Nos. 5,689,052; 5,500,365; 5,880,275); Bacillus thuringiensis toxic protein genes (see, e.g., U.S. Pat. Nos. 5,366,892; 5,747,450; 5,737,514; 5,723,756; 5,593,881; 6,555,655; 6,541,448; 6,538,109; Geiser, et al. (1986) Gene 48:109); and lectins (Van Damme et al. (1994) Plant Mol. Biol. 24:825). Nucleotide sequences encoding Bacillus thuringiensis (Bt) toxins from several subspecies have been cloned and recombinant clones have been found to be toxic to lepidopteran, dipteran and coleopteran insect larvae (for example, various delta-endotoxin genes such as CrylAa, Cry1Ab, Cry1Ac, Cry1B, Cry1C, Cry1D, Cry1Ea, Cry1Fa, Cry3A, Cry9A, Cry9C and Cry9B; as well as genes encoding vegetative insecticidal proteins such as Vip1, Vip2 and Vip3). A full list of Bt toxins can be found on the worldwide web at Bacillus thuringiensis Toxin Nomenclature Database maintained by the University of Sussex (see also, Crickmore et al. (1998) Microbiol. Mol. Biol. Rev. 62:807-813).

Polypeptides that are suitable for production in plants further include those that improve or otherwise facilitate the conversion of harvested plants and/or plant parts into a commercially useful product, including, for example, increased or altered carbohydrate content and/or distribution, improved fermentation properties, increased oil content, increased protein content, improved digestibility, and increased nutraceutical content, e.g., increased phytosterol content, increased tocopherol content, increased stanol content and/or increased vitamin content. Polypeptides of interest also include, for example, those resulting in, or contributing to, a reduced content of an unwanted component in a harvested crop, e.g., phytic acid, or sugar degrading enzymes. By “resulting in” or “contributing to” is intended that the polypeptide of interest can directly or indirectly contribute to the existence of a trait of interest (e.g., increasing cellulose degradation by the use of a heterologous cellulase enzyme).

In one embodiment, the polypeptide of interest contributes to improved digestibility for food or feed. Xylanases are hemicellulolytic enzymes that improve the breakdown of plant cell walls, which leads to better utilization of the plant nutrients by an animal. This leads to improved growth rate and feed conversion. Also, the viscosity of the feeds containing xylan can be reduced by xylanases. Heterologous production of xylanases in plant cells also can facilitate lignocellulosic conversion to fermentable sugars in industrial processing.

Numerous xylanases from fimgal and bacterial microorganisms have been identified and characterized (see, e.g., U.S. Pat. No. 5,437,992; Coughlin et al. (1993) “Proceedings of the Second TRICEL Symposium on Trichoderma reesei Cellulases and Other Hydrolases” Espoo; Souminen and Reinikainen, eds. (1993) Foundation for Biotechnical and Industrial Fermentation Research 8:125-135; U.S. Patent Publication No. 2005/0208178; and PCT Publication No. WO 03/16654). In particular, three specific xylanases (XYL-I, XYL-II, and XYL-III) have been identified in T. reesei (Tenkanen et al. (1992) Enzyme Microb. Technol, 14:566; Torronen et al. (1992) Bio/Technology 10:1461; and Xu et al, (1998) Appl. Microbiol. Biotechnol. 49:718).

In another embodiment, a polypeptide useful for the present invention can be a polysaccharide degrading enzyme. Plants producing such an enzyme may be useful for generating, for example, fermentation feedstocks for bioprocessing. In some embodiments, enzymes useful for a fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme or other glucoamylases.

Polysaccharide-degrading enzymes include: starch degrading enzymes such as alpha-amylases (EC 3.2.1.1), glucuronidases (E.C. 3.2.1.131), exo-1,4-alpha-D glucanases such as amyloglucosidases and glucoamylase (EC 3.2.1.3), beta-amylases (EC 3.2.1.2), alpha-glucosidases (EC 3.2.1.20), and other exo-amylases, starch debranching enzymes, such as a) isoamylase (EC 3.2.1.68), pullulanase (EC 3.2.1.41), and the like; b) cellulases such as exo-1,4-3-cellobiohydrolase (EC 3.2.1.91), exo-1,3-beta-D-glucanase (EC 3.2.1.39), beta-glucosidase (EC 3.2.1.21); c) L-arabinases, such as endo-1,5-alpha-L-arabinase (EC 3.2.1.99), alpha-arabinosidases (EC 3.2.1.55) and the like; d) galactanases such as endo-1,4-beta-D-galactanase (EC 3.2.1.89), endo-1,3-beta-D-galactanase (EC 3.2.1.90), alpha-galactosidase (EC 3.2.1.22), beta-galactosidase (EC 3.2.1.23) and the like; e) mannanases, such as endo-1,4-beta-D-mannanase (EC 3.2.1.78), beta-mannosidase (EC 3.2.1.25), alpha-mannosidase (EC 3.2.1.24) and the like; f) xylanases, such as endo-1,4-beta-xylanase (EC 3.2.1.8), beta-D-xylosidase (EC 3.2.1.37), 1,3-beta-D-xylanase, and the like; and g) other enzymes such as alpha-L-fucosidase (EC 3.2.1.51), alpha-L-rhamnosidase (EC 3.2.1.40), levanase (EC 3.2.1.65), inulanase (EC 3.2.1.7), and the like.

Further enzymes which may be used with the present invention include proteases, such as fungal and bacterial proteases. Fungal proteases include, but are not limited to, those obtained from Aspergillus, Trichoderma, Mucor and Rhizopus, such as A. niger, A. awamori, A. oryzae and M. miehei.

Other useful enzymes include, but are not limited to, hemicellulases, such as mannases and arabinofuranosidases (EC 3.2.1.55); ligninases; lipases (e.g., E.C. 3.1.1.3), glucose oxidases, pectinases, xylanases, transglucosidases, alpha 1,6 glucosidases (e.g., E.C. 3.2.1.20); cellobiohydrolases; esterases such as ferulic acid esterase (EC 3.1.1.73) and acetyl xylan esterases (EC 3.1.1.72); and cutinases (e.g. E.C. 3.1.1.74).

The nucleotide sequence can encode a reporter polypeptide (e.g., an enzyme), including but not limited to Green Fluorescent Protein, β-galactosidase, luciferase, alkaline phosphatase, the GUS gene encoding β-glucuronidase, and chloramphenicol acetyltransferase.

Where appropriate, the nucleotide sequence of interest may be optimized for increased expression in a transformed plant, e.g., by using plant preferred codons. Methods for synthetic optimization of nucleic acid sequences are available in the art. The nucleotide sequence of interest can be optimized for expression in a particular host plant or alternatively can be modified for optimal expression in monocots. See, e.g., EP 0 359 472, EP 0 385 962, WO 91/16432; Perlak et al., Proc. Natl. Acad. Sci. USA 88, 3324 (1991), and Murray et al., Nuc. Acids Res. 17, 477 (1989), and the like. Plant preferred codons can be determined from the codons of highest frequency in the proteins expressed in that plant.

Additional sequence modifications are known to enhance gene expression in a cellular host. These include elimination of sequences encoding spurious polyadenylation signals, exon-intron splice site signals, transposon-like repeats, and other such well-characterized sequences which may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

### III. Transgenic Plants, Plant Parts and Plant Cells.

The invention also provides transgenic plants, plant parts and plant cells comprising the nucleic acids, expression cassettes and vectors of the invention.

Accordingly, one aspect the invention provides a cell comprising a nucleic acid, expression cassette, and/or vector of the invention. The cell can be transiently or stably transformed with the nucleic acid, expression cassette and/or vector. Further, the cell can be a cultured cell, a cell obtained from a plant, plant part, or plant tissue, or a cell in situ in a plant, plant part or plant tissue. Cells can be from any suitable species, including plant (e.g. corn), bacterial, yeast, insect and/or mammalian cells. In representative embodiments, the cell is a plant cell or bacterial cell.

The invention also provides a plant part (including a plant tissue culture) comprising a nucleic acid, expression cassette, or vector of the invention. The plant part can be transiently or stably transformed with the nucleic acid, expression cassette and/or vector. Further, the plant part can be in culture, can be a plant part obtained from a plant, or a plant part in situ. In representative embodiments, the plant part comprises a cell of the invention.

Seed comprising the nucleic acid, expression cassette, or vector of the invention are also provided. In some embodiments of the present invention, the nucleic acid, expression cassette or vector is stably incorporated into the genome of the seed.

The invention also contemplates a transgenic plant comprising a nucleic acid, expression cassette, and/or vector of the invention. The plant can be transiently or stably transformed with a nucleic acid, expression cassette and/or vector comprising a recombinant promoter sequence of the invention. In representative embodiments, the plant comprises a cell and/or plant part of the invention (as described above).

Still further, the invention encompasses a crop comprising a plurality of the transgenic plants of the invention, as described herein. Nonlimiting examples of the types of crops comprising a plurality of transgenic plants of the invention include an agricultural field, a golf course, a residential lawn or garden, a public lawn or garden, a road side planting, an orchard, and/or a recreational field (e.g., a cultivated area comprising a plurality of the transgenic plants of the invention).

Products harvested from the plants of the invention are also provided. Nonlimiting examples of a harvested product include a seed, a leaf, a stem, a shoot, a fruit, flower, root, biomass (e.g., for biofuel production) and/or extract.

In some embodiments, a processed product produced from the harvested product is provided. Nonlimiting examples of a processed product include a polypeptide (e.g., a recombinant polypeptide), an extract, a medicinal product (e.g., artemicin as an antimalarial agent), a fiber or woven textile, a fragrance, dried fruit, a biofuel (e.g., ethanol), a tobacco product (e.g., cured tobacco, cigarettes, chewing tobacco, cigars, and the like), an oil (e.g., sunflower oil, corn oil, canola oil, and the like), a nut or seed butter, a flour or meal (e.g., wheat or rice flour, corn meal) and/or any other animal feed (e.g., soy, maize, barley, rice, alfalfa) and/or human food product (e.g., a processed wheat, maize, rice or soy food product).

### IV. Methods of Introducing Nucleic Acids.

The invention also provides methods of introducing a nucleic acid, expression cassette and/or vector as described herein into a target plant, plant part or plant cell (including callus cells or protoplasts), seed, plant tissue (including callus), and the like. In exemplary embodiments, the method is practiced to express a polynucleotide sequence of interest that is operably associated with a promoter comprising, consisting essentially of or consisting of a nucleotide sequence of the present invention (e.g., SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, and/or SEQ ID NO:45) as described herein. As described herein, the nucleotide sequences of the invention can be used in any combination. Thus, in some embodiments, a recombinant promoter can comprise, consist essentially of, or consist of one or more different nucleotide sequences of the invention, one or more of the same nucleotide sequence of the invention, or any combination thereof of the same or different nucleotide sequences of the invention. The invention further comprises plants (and progeny thereof), plant parts, seed, tissue culture (including callus) or cells, transiently or stably transformed with the nucleic acids, expression cassettes and/or vectors as described herein.

In representative embodiments, the invention provides a method of producing a plant comprising a nucleic acid of this invention, an expression cassette of this invention and/or a vector of this invention, the method comprising: introducing into a plant cell a nucleic acid of this invention, an expression cassette of this invention and/or a vector of this invention to produce a stably transformed plant cell; and regenerating a stably transformed plant from the plant cell.

In additional embodiments, the present invention provides a method of expressing a polynucleotide sequence of interest in a plant, the method comprising transforming a plant cell with an expression cassette or vector comprising a nucleic acid as described herein operably associated with a polynucleotide sequence of interest to produce a transformed plant cell, regenerating a stably transformed transgenic plant from the transformed plant cell, and expressing the polynucleotide sequence of interest in the plant.

Accordingly, in some embodiments, the present invention provides a method for directing juvenile root- and/or juvenile leaf-specific transcription of a polynucleotide of interest in a plant, the method comprising: introducing into a plant cell a nucleic acid comprising a recombinant promoter that comprises, consists essentially of, or consists of one or more nucleotide sequences of SEQ ID NO:1, SEQ ID NO:2, SEQ ID NO:3, SEQ ID NO:4, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and/or SEQ ID NO:20 (whereby the promoter is in operable association with said one or more nucleotide sequences of interest), wherein the promoter is operably linked to the polynucleotide of interest; and regenerating a plant from said plant cell, whereby the polynucleotide of interest is expressed in juvenile roots and/or juvenile leaves. In some particular embodiments, the recombinant promoter comprises, consists essentially of, or consists of the nucleotide sequence of SEQ ID NO:1, wherein said promoter is further operably associated with a polynucleotide of interest that includes, but is not limited to, a root-specific polynucleotide, a leaf specific polynucleotide, and/or a polynucleotide related to seed development. In other embodiments, the recombinant promoter comprises, consists essentially of, or consists of the nucleotide sequence of SEQ ID NO:1, wherein said promoter is further operably associated with a polynucleotide of interest that includes, but is not limited to polynucleotides encoding low affinity and high affinity nitrate transporters (e.g., Nrt family), ammonium transporters (e.g., AMT family), amino acid and peptide transporters, polypeptides involved in nitrogen assimilation (e.g., Glutamine Synthases Gln1-5), and/or transporters and other polypeptides involved in assimilation of mineral nutrients that act in concert with nitrogen including, but not limited to, phosphate, sulfur, potassium, silicon and the micronutrients including, but not limited to, iron, manganese, zinc, copper, boron and molybdenum.

In other embodiments, the present invention provides a method for directing juvenile to adult transition leaf-specific transcription of a polynucleotide of interest in a plant, the method comprising: introducing into a plant cell a nucleic acid comprising a recombinant promoter that comprises, consists essentially of, or consists of the nucleotide sequence of SEQ ID NO:5 and/or SEQ ID NO:6, wherein the promoter is operably linked to the polynucleotide of interest; and regenerating a plant from said plant cell, whereby the polynucleotide of interest is expressed in juvenile to adult transition leaves.

In still other embodiments, the invention provides a method for directing adult to post-flowering leaf-specific transcription of a polynucleotide of interest in a plant, the method comprising: introducing into a plant cell a nucleic acid comprising a recombinant promoter that comprises, consists essentially of, consists of the nucleotide sequence of SEQ ID NO:8, wherein the promoter is operably linked to the polynucleotide of interest; and regenerating a plant from said plant cell, whereby the polynucleotide of interest is expressed in adult to post-flowering leaves.

A further embodiment of the invention provides a method for directing coleoptile-specific transcription of a polynucleotide of interest in a plant, the method comprising: introducing into a plant cell a nucleic acid comprising a recombinant promoter that comprises, consists essentially of, consists of a nucleotide sequence of SEQ ID NO:21 and/or SEQ ID NO:22, wherein the promoter is operably linked to the polynucleotide of interest; and regenerating a plant from said plant cell, whereby the polynucleotide of interest is expressed in coleoptiles.

In additional embodiments of the invention a method for directing juvenile leaf-specific transcription of a polynucleotide of interest in a plant is provided, the method comprising: introducing into a plant cell a nucleic acid comprising a recombinant promoter that comprises, consists essentially of, consists of one or more nucleotide sequences of SEQ ID NO:23, SEQ ID NO:30, and/or SEQ ID NO:31, wherein the promoter is operably linked to the polynucleotide of interest; and regenerating a plant from said plant cell, whereby the polynucleotide is expressed in juvenile leaves.

In some embodiments of the invention, a method for directing juvenile root-specific transcription of a polynucleotide of interest in a plant is provided, the method comprising: introducing into a plant cell a nucleic acid comprising a recombinant promoter that comprises, consists essentially of, consists of one or more nucleotide sequences of SEQ ID NO:24, SEQ ID NO:25, SEQ ID NO:26, SEQ ID NO:32 and/or SEQ ID NO:33, wherein the promoter is operably linked to the polynucleotide of interest; and regenerating a plant from said plant cell, whereby the polynucleotide of interest is expressed in juvenile roots.

In other embodiments of the invention, a method for directing adult leaf-specific transcription of a polynucleotide of interest in a plant is provided, the method comprising: introducing into a plant cell a nucleic acid comprising a recombinant promoter that comprises, consists essentially of, consists of one or more nucleotide sequences of SEQ ID NO:34, SEQ ID NO:35, and/or SEQ ID NO:36, wherein the promoter is operably linked to the polynucleotide of interest; and regenerating a plant from said plant cell, whereby the polynucleotide of interest is expressed in adult leaves.

In still other embodiments of the invention, a method for directing adult root-specific transcription of a polynucleotide of interest in a plant is provided, the method comprising: introducing into a plant cell a nucleic acid comprising a recombinant promoter that comprises, consists essentially of, consists of one or more nucleotide sequences of SEQ ID NO:37, SEQ ID NO:38, SEQ ID NO:39, SEQ ID NO:40, and/or SEQ ID NO:41, wherein the promoter is operably linked to the polynucleotide of interest; and regenerating a plant from said plant cell, whereby the polynucleotide of interest is expressed in adult roots.

In additional embodiments of the invention, a method for directing pre-flowering leaf-specific transcription of a polynucleotide of interest in a plant is provided, the method comprising: introducing into a plant cell a nucleic acid comprising a recombinant promoter that comprises, consists essentially of, consists of the nucleotide sequence of SEQ ID NO:42, wherein the promoter is operably linked to the polynucleotide of interest; and regenerating a plant from said plant cell, whereby the polynucleotide of interest is expressed in pre-flowering leaves.

In some embodiments, the present invention provides a method for directing reproductive stage leaf-specific transcription of a polynucleotide of interest in a plant, the method comprising: introducing into a plant cell a nucleic acid comprising a recombinant promoter that comprises, consists essentially of, consists of the nucleotide sequence of SEQ ID NO:43, wherein the promoter is operably linked to the polynucleotide of interest; and regenerating a plant from said plant cell, whereby the polynucleotide of interest is expressed in reproductive stage leaves.

In other embodiments, the present invention provides a method for directing reproductive stage root-specific transcription of a polynucleotide of interest in a plant, the method comprising: introducing into a plant cell a nucleic acid comprising a recombinant promoter that comprises, consists essentially of, consists of the nucleotide sequence of SEQ ID NO:44 and/or SEQ ID NO:45, wherein the promoter is operably linked to the polynucleotide of interest; and regenerating a plant from said plant cell, whereby the polynucleotide of interest is expressed in reproductive stage roots.

In still other embodiments, the present invention provides a method for directing root-specific transcription of a polynucleotide of interest in a plant, the method comprising: introducing into a plant cell a nucleic acid comprising a recombinant promoter that comprises, consists essentially of, consists of one or more nucleotide sequences of SEQ ID NO:7, SEQ ID NO:27, SEQ ID NO: 28 and/or SEQ ID NO:29, wherein the promoter is operably linked to the polynucleotide of interest; and regenerating a plant from said plant cell, whereby the polynucleotide of interest is expressed in roots. In some representative embodiments, the promoter comprises, consists essentially of consists of the nucleotide sequence of SEQ ID NO:27 and the polynucleotide of interest is expressed in seminal roots. In other embodiments, the promoter comprises, consists essentially of, consists of the nucleotide sequence of SEQ ID NO:28 and/or SEQ ID NO:29 and the polynucleotide of interest is expressed in nodal roots.

Thus, the present invention can be advantageously practiced to effect the expression of a polynucleotide of interest operably associated with a recombinant promoter as described herein, such that the polynucleotide sequence is expressed at one or more desired stages of plant development (e.g., juvenile, adult, pre-flowering, post-flowering, and the like) and/or in one or more specific tissues (e.g., leaf-, root- and/or root hair-specific expression). As described herein, the nucleotide sequences of the invention can be used in any combination. Further, the nucleotide sequences of the present invention can be used in combination with other promoter elements that confer responsiveness to, for example, environmental cues. Thus, in some representative embodiments, by regulating gene expression in a developmental manner (for example, the juvenile to adult stage transition), the nucleotide sequences of the present invention can be used, for example, to maintain a plant in a juvenile stage in combination with motifs that regulate gene expression in response to, for example, drought, thereby making the plant drought tolerant at the juvenile stage of development.

The invention further encompasses transgenic plants (and progeny thereof), plant parts, and plant cells produced by the methods of the invention, wherein the transgenic plants (and progeny thereof) comprise nucleic acid (and/or expression cassette and/or vector) of the invention that comprises a recombinant promoter comprising a nucleotide sequence of the invention (e.g., SEQ ID NOs:1-45), wherein the recombinant promoter confers developmental stage specific or tissue specific transcription on a polynucleotide of interest operably linked to said promoter. Also provided by the invention are seed produced from the inventive transgenic plants, wherein the seed comprise a nucleic acid, expression cassette and/or vector as described herein stably incorporated into the genome, wherein the nucleic acid, expression cassette and/or vector comprise a recombinant promoter comprising a nucleotide sequence of the invention (e.g., SEQ ID NOs:1-45), wherein the recombinant promoter confers developmental stage specific or tissue specific transcription on a polynucleotide of interest operably linked to said promoter.

Methods of introducing nucleic acids, transiently or stably, into plants, plant tissues, cells, protoplasts, seed, callus and the like are known in the art. Stably transformed nucleic acids can be incorporated into the genome. Exemplary transformation methods include biological methods using viruses and bacteria (e.g., Agrobacterium), physicochemical methods such as electroporation, floral dip methods, ballistic bombardment, microinjection, and the like. Other transformation technology includes the whiskers technology that is based on mineral fibers (see e.g., U.S. Pat. Nos. 5,302,523 and 5,464,765) and pollen tube transformation.

Other exemplary transformation methods include, without limitation, calcium-phosphate-mediated transformation, cyclodextrin-mediated transformation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Thus, in some particular embodiments, the method of introducing into a plant, plant part, plant tissue, plant cell, protoplast, seed, callus and the like comprises bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethyleneglycol-mediated transformation, any other electrical, chemical, physical and/or biological mechanism that results in the introduction of nucleic acid into the plant, plant part and/or cell thereof, or a combination thereof.

In one form of direct transformation, the vector is microinjected directly into plant cells by use of micropipettes to mechanically transfer the recombinant DNA (Crossway, Mol. Gen. Genetics 202: 179 (1985)).

In another protocol, the genetic material is transferred into the plant cell using polyethylene glycol (Krens, et al. Nature 296, 72 (1982)).

In still another method, protoplasts are fused with minicells, cells, lysosomes, or other fusible lipid-surfaced bodies that contain the nucleotide sequence to be transferred to the plant (Fraley, et al., Proc. Natl, Acad. Sci. USA 79, 1859 (1982)).

Nucleic acids may also be introduced into the plant cells by electroporation (Fromm et al., Proc. Natl. Acad. Sci. USA 82, 5824 (1985)). In this technique, plant protoplasts are electroporated in the presence of nucleic acids comprising the expression cassette. Electrical impulses of high field strength reversibly permeabilize biomembranes allowing the introduction of the nucleic acid. Electroporated plant protoplasts reform the cell wall, divide and regenerate. One advantage of electroporation is that large pieces of DNA, including artificial chromosomes, can be transformed by this method.

Ballistic transformation typically comprises the steps of: (a) providing a plant material as a target; (b) propelling a microprojectile carrying the heterologous nucleotide sequence at the plant target at a velocity sufficient to pierce the walls of the cells within the target and to deposit the nucleotide sequence within a cell of the target to thereby provide a transformed target. The method can further include the step of culturing the transformed target with a selection agent and, optionally, regeneration of a transformed plant. As noted below, the technique may be carried out with the nucleotide sequence as a precipitate (wet or freeze-dried) alone, in place of the aqueous solution containing the nucleotide sequence.

Any ballistic cell transformation apparatus can be used in practicing the present invention. Exemplary apparatus are disclosed by Sandford et al. (Particulate Science and Technology 5, 27 (1988)), Klein et al. (Nature 327, 70 (1987)), and in EP 0 270 356. Such apparatus have been used to transform maize cells (Klein et al., Proc. Natl. Acad. Sci. USA 85, 4305 (1988)), soybean callus (Christou et al., Plant Physiol. 87, 671 (1988)), McCabe et al., BioTechnology 6, 923 (1988), yeast mitochondria (Johnston et al., Science 240, 1538 (1988)), and Chlamydomonas chloroplasts (Boynton et al., Science 240, 1534 (1988)).

Alternately, an apparatus configured as described by Klein et al. (Nature 70, 327 (1987)) may be utilized. This apparatus comprises a bombardment chamber, which is divided into two separate compartments by an adjustable-height stopping plate. An acceleration tube is mounted on top of the bombardment chamber. A macroprojectile is propelled down the acceleration tube at the stopping plate by a gunpowder charge. The stopping plate has a borehole formed therein, which is smaller in diameter than the microprojectile. The macroprojectile carries the microprojectile(s), and the macroprojectile is aimed and fired at the borehole. When the macroprojectile is stopped by the stopping plate, the microprojectile(s) is propelled through the borehole. The target is positioned in the bombardment chamber so that a microprojectile(s) propelled through the bore hole penetrates the cell walls of the cells in the target and deposit the polynucleotide sequence of interest carried thereon in the cells of the target. The bombardment chamber is partially evacuated prior to use to prevent atmospheric drag from unduly slowing the microprojectiles. The chamber is only partially evacuated so that the target tissue is not desiccated during bombardment. A vacuum of between about 400 to about 800 millimeters of mercury is suitable.

In alternate embodiments, ballistic/biolistic transformation is achieved without use of microprojectiles. For example, an aqueous solution containing the polynucleotide sequence of interest as a precipitate may be carried by the macroprojectile (e.g., by placing the aqueous solution directly on the plate-contact end of the macroprojectile without a microprojectile, where it is held by surface tension), and the solution alone propelled at the plant tissue target (e.g., by propelling the macroprojectile down the acceleration tube in the same manner as described above). Other approaches include placing the nucleic acid precipitate itself (“wet” precipitate) or a freeze-dried nucleotide precipitate directly on the plate-contact end of the macroprojectile without a microprojectile. In the absence of a microprojectile, it is believed that the nucleotide sequence must either be propelled at the tissue target at a greater velocity than that needed if carried by a microprojectile, or the nucleotide sequenced caused to travel a shorter distance to the target (or both).

It particular embodiments, the nucleotide sequence is delivered by a microprojectile. The microprojectile can be formed from any material having sufficient density and cohesiveness to be propelled through the cell wall, given the particle's velocity and the distance the particle must travel. Non-limiting examples of materials for making microprojectiles include metal, glass, silica, ice, polyethylene, polypropylene, polycarbonate, and carbon compounds (e.g., graphite, diamond). Non-limiting examples of suitable metals include tungsten, gold, and iridium. The particles should be of a size sufficiently small to avoid excessive disruption of the cells they contact in the target tissue, and sufficiently large to provide the inertia required to penetrate to the cell of interest in the target tissue. Particles ranging in diameter from about one-half micrometer to about three micrometers are suitable. Particles need not be spherical, as surface irregularities on the particles may enhance their carrying capacity.

The nucleotide sequence may be immobilized on the particle by precipitation. The precise precipitation parameters employed will vary depending upon factors such as the particle acceleration procedure employed, as is known in the art. The carrier particles may optionally be coated with an encapsulating agents such as polylysine to improve the stability of nucleotide sequences immobilized thereon, as discussed in EP 0 270 356 (column 8).

Alternatively, plants may be transformed using Agrobacterium tumefaciens or Agrobacterium rhizogenes. Agrobacterium-mediated nucleic acid transfer exploits the natural ability of A. tumefaciens and A. rhizogenes to transfer DNA into plant chromosomes. Agrobacterium is a plant pathogen that transfers a set of genes encoded in a region called T-DNA of the Ti and Ri plasmids of A. tumefaciens and A. rhizogenes, respectively, into plant cells. The typical result of transfer of the Ti plasmid is a tumorous growth called a crown gall in which the T-DNA is stably integrated into a host chromosome. Integration of the Ri plasmid into the host chromosomal DNA results in a condition known as “hairy root disease”. The ability to cause disease in the host plant can be removed by deletion of the genes in the T-DNA without loss of DNA transfer and integration. The DNA to be transferred is attached to border sequences that define the end points of an integrated T-DNA.

Transfer by means of engineered Agrobacterium strains has become routine for many dicotyledonous plants. Some difficulty has been experienced, however, in using Agrobacterium to transform monocotyledonous plants, in particular, cereal plants. However, Agrobacterium mediated transformation has been achieved in several monocot species, including cereal species such as rye, maize (Rhodes et al., Science 240, 204 (1988)), and rice (Hiei et al., (1994) Plant 6:271).

While the following discussion will focus on using A. tumefaciens to achieve gene transfer in plants, those skilled in the art will appreciate that this discussion also applies to A. rhizogenes. Transformation using A. rhizogenes has developed analogously to that of A. tumefaciens and has been successfully utilized to transform, for example, alfalfa, Solanum nigrum L., and poplar (U.S. Pat. No. 5,777,200 to Ryals et al.). As described by U.S. Pat. No. 5,773,693 to Burgess et al., it is preferable to use a disarmed A. tumefaciens strain (as described below), however, the wild-type A. rhizogenes may be employed. An illustrative strain of A. rhizogenes is strain 15834.

In particular protocols, the Agrobacterium strain is modified to contain the nucleotide sequences to be transferred to the plant. The nucleotide sequence to be transferred is incorporated into the T-region and is typically flanked by at least one T-DNA border sequence, optionally two T-DNA border sequences. A variety of Agrobacterium strains are known in the art particularly, and can be used in the methods of the invention. See, e.g., Hooykaas, Plant Mot. Biol. 13, 327 (1989); Smith et al., Crop Science 35, 301 (1995); Chilton, Proc. Natl. Acad. Sci. USA 90, 3119 (1993); Mollony et al., Monograph Theor. Appl. Genet NY 19, 148 (1993); Ishida et al., Nature Biotechnol. 14, 745 (1996); and Komari et al., The Plant Journal 10, 165 (1996).

In addition to the T-region, the Ti (or Ri) plasmid contains a vir region. The vir region is important for efficient transformation, and appears to be species-specific.

Two exemplary classes of recombinant Ti and Ri plasmid vector systems are commonly used in the art. In one class, called “cointegrate,” the shuttle vector containing the gene of interest is inserted by genetic recombination into a non-oncogenic Ti plasmid that contains both the cis-acting and trans-acting elements required for plant transformation as, for example, in the PMLJ1 shuttle vector of DeBlock et al., EMBO J3, 1681 (1984), and the non-oncogenic Ti plasmid pGV2850 described by Zambryski et al., EMBOJ2, 2143 (1983). In the second class or “binary” system, the gene of interest is inserted into a shuttle vector containing the cis-acting elements required for plant transformation. The other necessary functions are provided in trans by the non-oncogenic Ti plasmid as exemplified by the pBIN19 shuttle vector described by Bevan, Nucleic Acids Research 12, 8711 (1984), and the non-oncogenic Ti plasmid PAL4404 described by Hoekma, et al., Nature 303, 179 (1983).

Binary vector systems have been developed where the manipulated disarmed T-DNA carrying the heterologous polynucleotide sequence of interest and the vir functions are present on separate plasmids. In this manner, a modified T-DNA region comprising foreign DNA (the nucleic acid to be transferred) is constructed in a small plasmid that replicates in E. coli. This plasmid is transferred conjugatively in a tri-parental mating or via electroporation into A. tumefaciens that contains a compatible plasmid with virulence gene sequences. The vir functions are supplied in trans to transfer the T-DNA into the plant genome. Such binary vectors are useful in the practice of the present invention.

In particular embodiments of the invention, super-binary vectors are employed. See, e.g., U.S. Pat. No. 5,591,615 and EP 0 604 662. Such a super-binary vector has been constructed containing a DNA region originating from the hypervirulence region of the Ti plasmid pTiBo542 (Jin et al., J Bacteriol. 169, 4417 (1987)) contained in a super-virulent A. tumefaciens A281 exhibiting extremely high transformation efficiency (Hood et al., Biotechnol. 2, 702 (1984); Hood et al., J. Bacteriol. 168, 1283 (1986); Komari et al., J Bacteriol. 166, 88 (1986); Jin et al., J. Bacteriol. 169, 4417 (1987); Komari, Plant Science 60, 223 (1987); ATCC Accession No. 37394.

Exemplary super-binary vectors known to those skilled in the art include pTOK162 (Japanese patent Appl. (Kokai) No. 4-222527, EP 504,869, EP 604,662, and U.S. Pat. No. 5,591,616) and pTOK233 (Komari, Plant Cell Reports 9, 303 (1990); Ishida et al., Nature Biotechnology 14, 745 (1996)). Other super-binary vectors may be constructed by the methods set forth in the above references. Super-binary vector pTOK162 is capable of replication in both E. coli and in A. tumefaciens. Additionally, the vector contains the virB, virC and virG genes from the virulence region of pTiBo542. The plasmid also contains an antibiotic resistance gene, a selectable marker gene, and the nucleic acid of interest to be transformed into the plant. The nucleic acid to be inserted into the plant genome is typically located between the two border sequences of the T region. Super-binary vectors of the invention can be constructed having the features described above for pTOK162. The T-region of the super-binary vectors and other vectors for use in the invention are constructed to have restriction sites for the insertion of the genes to be delivered. Alternatively, the DNA to be transformed can be inserted in the T-DNA region of the vector by utilizing in vivo homologous recombination. See, Herrera-Esterella et al., EMBO J. 2, 987 (1983); Horch et al., Science 223, 496 (1984). Such homologous recombination relies on the fact that the super-binary vector has a region homologous with a region of pBR322 or other similar plasmids. Thus, when the two plasmids are brought together, a desired gene is inserted into the super-binary vector by genetic recombination via the homologous regions.

In plants stably transformed by Agrobacteria-mediated transformation, the nucleotide sequence of interest is incorporated into the plant nuclear genome, typically flanked by at least one T-DNA border sequence and generally two T-DNA border sequences.

Plant cells may be transformed with Agrobacteria by any means known in the art, e.g., by co-cultivation with cultured isolated protoplasts, or transformation of intact cells or tissues. The first uses an established culture system that allows for culturing protoplasts and subsequent plant regeneration from cultured protoplasts. Identification of transformed cells or plants is generally accomplished by including a selectable marker in the transforming vector, or by obtaining evidence of successful bacterial infection.

Methods of introducing a nucleic acid into a plant can also comprise in vivo modification of genetic material, methods for which are known in the art. For example, in vivo modification can be used to insert a nucleic acid comprising a promoter sequence of the invention into the plant genome.

Suitable methods for in vivo modification include the techniques described in Gao et. al., Plant J. 61, 176 (2010); Li et al., Nucleic Acids Res. 39, 359 (2011); U.S. Pat. Nos. 7,897,372 and 8,021,867; U.S. Patent Publication No. 2011/0145940 and in International Patent Publication Nos. WO 2009/114321, WO 2009/134714 and WO 2010/079430. For example, one or more transcription affector-like nucleases (TALEN) and/or one or more meganucleases may be used to incorporate an isolated nucleic acid comprising a promoter sequence of the invention into the plant genome. In representative embodiments, the method comprises cleaving the plant genome at a target site with a TALEN and/or a meganuclease and providing a nucleic acid that is homologous to at least a portion of the target site and further comprises a nucleotide sequence of this invention (e.g., SEQ ID NOs:1-45), such that homologous recombination occurs and results in the insertion of the nulceotide sequence of the invention into the genome.

Protoplasts, which have been transformed by any method known in the art, can also be regenerated to produce intact plants using known techniques.

Plant regeneration from cultured protoplasts is described in Evans et al., Handbook of Plant Cell Cultures, Vol. 1: (MacMilan Publishing Co. New York, 1983); and Vasil I. R. (ed.), Cell Culture and Somatic Cell Genetics of Plants, Acad. Press, Orlando, Vol. I, 1984, and Vol. II, 1986). Essentially all plant species can be regenerated from cultured cells or tissues, including but not limited to, all major species of sugar-cane, sugar beet, cotton, fruit trees, and legumes.

Means for regeneration vary from species to species of plants, but generally a suspension of transformed protoplasts or a petri plate containing transformed explants is first provided. Callus tissue is formed and shoots may be induced from callus and subsequently root. Alternatively, somatic embryo formation can be induced in the callus tissue. These somatic embryos germinate as natural embryos to form plants. The culture media will generally contain various amino acids and plant hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

The regenerated plants are transferred to standard soil conditions and cultivated in a conventional manner. The plants are grown and harvested using conventional procedures.

Alternatively, transgenic plants may be produced using the floral dip method (See, e.g., Clough and Bent (1998) Plant Journal 16:735-743, which avoids the need for plant tissue culture or regeneration. In one representative protocol, plants are grown in soil until the primary inflorescence is about 10 cm tall. The primary inflorescence is cut to induce the emergence of multiple secondary inflorescences. The inflorescences of these plants are typically dipped in a suspension of Agrobacterium containing the vector of interest, a simple sugar (e.g., sucrose) and surfactant. After the dipping process, the plants are grown to maturity and the seeds are harvested. Transgenic seeds from these treated plants can be selected by germination under selective pressure (e.g., using the chemical bialaphos). Transgenic plants containing the selectable marker survive treatment and can be transplanted to individual pots for subsequent analysis. See Bechtold, N. and Pelletier, G. Methods Mol Biol 82, 259-266 (1998); Chung, M. H. et al. Transgenic Res 9, 471-476 (2000); Clough, S. J. and Bent, A. F. Plant J 16, 735-743 (1998); Mysore, K. S. et al. Plant J 21, 9-16 (2000); Tague, B. W. Transgenic Res 10, 259-267 (2001); Wang, W. C. et al. Plant Cell Rep 22, 274-281 (2003); Ye, G. N. et al. Plant J., 19:249-257 (1999).

The particular conditions for transformation, selection and regeneration can be optimized by those of skill in the art. Factors that affect the efficiency of transformation include the species of plant, the target tissue or cell, composition of the culture media, selectable marker genes, kinds of vectors, and light/dark conditions. Therefore, these and other factors may be varied to determine an optimal transformation protocol for any particular plant species. It is recognized that not every species will react in the same manner to the transformation conditions and may require a slightly different modification of the protocols disclosed herein. However, by altering each of the variables, an optimum protocol can be derived for any plant species.

Further, the genetic properties engineered into the transgenic seeds and plants, plant parts, and/or plant cells of the present invention described herein can be passed on by sexual reproduction or vegetative growth and therefore can be maintained and propagated in progeny plants. Generally, maintenance and propagation make use of known agricultural methods developed to fit specific purposes such as harvesting, sowing or tilling.

The present invention is more particularly described in the following examples that are intended as illustrative only since numerous modifications and variations therein will be apparent to those skilled in the art.

### EXAMPLES

### Example 1

### Tissue and/or Developmental Stage-Specific Expression of Nitrogen-Related Genes in Maize

Microarray analysis was used to characterize the expression of genes associated with nitrogen transport and assimilation in various maize tissues at different stages of plant development and then to identify over-represented cis-acting promoter motifs corresponding to genes that are up-regulated in particular tissues/stages.

### Materials and Methods

Plant Growth and Tissue Harvest.

Proprietary hybrid seeds (SRG150, Syngenta Biotechnology, Inc., Durham, N.C.) were grown in a greenhouse during the summer of 2007 at the University of Guelph in Ontario, Canada. Plants were grown semi-hydroponically in pots containing Turface® clay soil conditioner (PROFILE Products, LLC, Buffalo Grove, Ill.) at 50% relative humidity with 16 hours of light (about 600 μmol m⁻² s⁻¹) at 28° C. and eight hours of dark at 23° C. Plants were watered with a nutrient solution containing: 0.4 g/L 28-14-14 fertilizer, 0.4 g/L 15-15-30 fertilizer, 0.2 g/L NH₄NO₃, 0.4 g/L of MgSO4.7H₂O and 0.03 g/L of micronutrient mix (S, Co, Cu, Fe, Mn, Mo and Zn). Three biological replicates per tissue/stage were harvested, always at about 11:00 am.

### Microarray Analysis and Normalization.

Total RNA was isolated using Trizol reagent and RNeasy column to purify the extracts from protein and perform a DNAse treatment (Dallas et al, 2005; BMC Genomics. 2005; 6:59; doi: 10.1186/1471-2164-6-59; Bi et al. BMC Genomics 8:281(2007)). Messenger RNA (mRNA) was isolated from 50 maize tissues at different stages of plant development, ranging from seedling emergence (Ve) to 31 days after pollination (31DAP) (three biological replicates per tissue/stage) (Table 1). mRNA was hybridized onto 150 customized maize Affymetrix 82K Unigene arrays (Affymetrix, Inc., Santa Clara, Calif.) as described by Wagner and Radelof, J. BIOTECH. 129:628 (2007). Gene expression was normalized using the RMA method⁵² from Bioconductor⁵³ as described by Gentleman et al., GENOME BIOL. 5:R80 (2004), and Irizarry et al., NUCLEIC ACIDS RES. 31:e15 (2003).

TABLE 1
Developmental stages and tissues sampled.

| | | Visible | Detail of the harvested |
|---|---|---|---|
| Stage | Tissue/Organ | Leaves | sample |
| VE | leaf | 0 | coleoptile |
| VE | seminal root | 0 | root |
| V1 | leaf | 2 | 1st & 2nd leaf |
| V1 | seminal root | 2 | root |
| V2 | seminal root | 4 | seminal root |
| V2 | nodal root | 4 | nodal root |
| V2 | stalk | 4 | stalk |
| V2 | leaf | 4 | leaf (actively growing leaf- |
| | | | 4th leaf) |
| V4 | tassel | 6 | 1 mm tassel meristem & 1 mm |
| | | | uppermost stem below tassel |
| V5 | seminal root | 8 | seminal root |
| V5 | nodal root | 8 | nodal root |
| V5 | stalk | 8 | stalk below tassel (2 cm) |
| V5 | leaf | 8 | leaf (actively growing leaf- |
| | | | 8th leaf, 15 cm including tip) |
| V5 | tassel | 8 | tassel 3-5 mm |
| V7 | ear | 12 | tassel 2 cm |
| V7 | tassel | 12 | top ear shoot |
| V8~V9 | tassel | 13~14 | tassel 12~14 cm |
| V8~V9 | ear | 13~14 | top ear 3~5 mm |
| V10~V11 | tassel | 15~16 | top 10 cm of tassel (~20 cm) |
| V10~V11 | ear | 15~16 | top ear 1~1.5 cm |
| V13~V15 | tassel | 15~16 | spikelet of tassel (~22 cm) |
| V13~V15 | ear | 15~16 | top ear 3~3.5 cm |
| V15~V16 | floret | 15~16 | top ear(5 cm) floret |
| V15~V16 | cob | 15~16 | top ear (5 cm)cob |
| V15~V16 | silk | 15~16 | top ear (5 cm)silk |
| V15~V16 | tassel | 15~16 | spikelet of tassel (top 10 cm) |
| VT | anthers | 15~16 | anther |
| R1 | ovule | 15~16 | R1-ovule of top ear |
| R1 | cob | 15~16 | R1-cob of top ear |
| R1 | silk | 15~16 | R1-silk of top ear |
| R1 | husk | 15~16 | R1-most inner husk of top ear |
| R1 | leaf | 15~16 | R1-15 cm tip of 2nd leaf above |
| | | | top ear |
| R1 | nodal root | 15~16 | R1-adult root |
| R1 | stalk | 15~16 | R1-15 cm stalk below tassel |
| 5DAP | ovule | 15~16 | ovule of top ear |
| 5DAP | cob | 15~16 | cob of top ear |
| 10DAP | embryo | 15~16 | embryo of top ear |
| 10DAP | endosperm | 15~16 | endosperm of top ear |
| 10DAP | leaf | 15~16 | 15 cm tip of 2nd leaf above top |
| | | | ear |
| 17DAP | embryo | 15~16 | embryo of top ear |
| 17DAP | endosperm | 15~16 | endosperm of top ear |
| 17DAP | pericarp | 15~16 | pericarp of top ear |
| 17DAP | leaf | 15~16 | 15 cm tip of 2nd leaf above top |
| | | | ear |
| 24DAP | leaf | 15~16 | 15 cm tip of 2nd leaf above top |
| | | | ear |
| 24DAP | nodal root | 15~16 | root |
| 24DAP | pericarp | 15~16 | pericarp of top ear |
| 24DAP | embryo | 15~16 | embryo of top ear |
| 24DAP | endosperm | 15~16 | endosperm of top ear |
| 31DAP | leaf | 15~16 | 15 cm tip of 2nd leaf above top |
| | | | ear |
| 31DAP | embryo | 15~16 | embryo of top ear |

Identification of Probe Sets Corresponding to Nitrogen-Related Genes.

Array probe sets corresponding to genes associated with nitrogen transport and assimilation were identified using three methods. First, probe sets with no expression (relative expression <100) in any of the 150 microarray experiments (26,989 probe sets) were removed. Next, because the probe sets were designed from the maize Unigene set, the corresponding original Genbank sequences were used to identify potential gene matches via nucleotide BLAST searches against the B73 maize genome (release 4a.53, MaizeSequence.org). Each probe set was composed of 16 probes of 25 nucleotides each. If 75% (12/16) or more of the probes in a given probe set matched the same gene model, that probe set was identified as a match for that gene. If fewer than 75% of the probes in a given probe set matched the same gene model, the probes were considered as partial matches for that gene; a probe was required to have 85% sequence identity with that gene model in order to be considered a valid match of that gene. A total of 33,664 probe sets matching to unique gene models were mapped using these steps. Exonerate alignment (Slater and Birney, BMC BIOINFORMATICS 6:31 (2005)) was used to annotate an additional 9,919 probe sets. Nucleotide BLAST was not successful in identifying expressed sequence tag (EST) matches because of the biases created by gene model issues related to gene-calling software (GeneBuilder or FGENESH). The remaining 12,089 probe sets showed expression (relative expression >100) on at least one microarray, but did not map to the maize genome. After elimination of the probe sets with no expression and cross-hybridizing or redundant probe sets, there were 22,787 high-quality annotated probe sets. Additional methods were used. Specifically, the array probe sequences were re-screened for matches with EST sequences from NCBI using BLAST. Finally, nitrogen uptake and assimilation keywords were used to search gene annotations and protein domains in the B73 maize genome (release 4a.53, MaizeSequence.org), and the search results were each matched to a Genbank protein with the highest homology with the microarray probe set.

Identification of Gene Expression Clusters.

Gene expression clusters were identified using K-means clustering, as described by Hartigan and Wong, J. ROYAL STAT. SOC. SERIES C (APPLIED STAT.) 28:100 (1979).

Statistical Analysis of Tissue-Specific Gene Expression.

Tissue-specific and tissue-selective gene expression was analyzed using the Intersection Union Test (IUT; Berger and Hsu, STAT. SCI. 11:283 (1996)) using R coding modified from BayesianlUT (Katholieke Universiteit Leuven, Leuven, Netherlands; available at ppw.kuleuven.be/okp/software/BayesianlUT/; Van Deun et al., BIOINFORMATICS 25:2588 (2009)). The null hypothesis tested by IUT was that expression of a given gene in the target tissue was not significantly different from expression of that gene in one or more other tissues. The alternate hypothesis tested by IUT was that expression of a given gene was higher in the target tissue than in any other tissue. A gene was determined to be tissue-specific if all possible pairwise t-tests between the target tissue and the other tissues analyzed indicated a significant difference in mean gene expression. Sidak's adjustment (Sidak, J. AMER. STAT. ASSOC. 62:626 (1967)), which is equivalent to the Bonferroni correction, was used to correct the multiple testing methodology of the custom R package.

Statistical Analysis of Differential Gene Expression.

Differential gene expression was measured by fitting linear models to the data using the Limma Package from Bioconductor, as described by Smyth in Gentleman et al., BIOINFORMATICS AND COMPUTATIONAL BIOLOGY SOLUTIONS USING R AND BIOCONDUCTOR (2005). The linear models were adjusted using the empirical Bayesian method (modified t-test) form the Limma Package and were corrected for multiple testing as described by Benjamini and Hochberg, J. ROYAL STAT. SOC. SERIES B 57:289 (1995). The significance threshold of the adjusted p-value was set at 0.05.

Promoter Motif Discovery.

The promoter region (−500 to +1, relative to the ATG start codon) of each of the genes corresponding to a tissue-specific or tissue-selective array probe was analyzed for over-represented de novo cis-acting motifs (≧6 bp) using Promzea (available at promzea.org), a Perl-based motif discovery program that filters and combines the results of three motif discovery tools: Weeder (Pavesi et al., BMC BIOINFORMATICS 8:46 (2007)), MEME (Bailey et al., PROC. SECOND INTL. CONF. INTELLIGENT SYS. MOL. BIOL. 28-36 (1994)) and BioProspector (Liu et al., PAC. SYMP. BIOCOMPUTING 2001:127 (2001))—and statistically validates identified motifs using the hypergeometric test or the binomial test. All significant motifs identified in the aforementioned search were compared to previously defined motifs in the Athamap (Bulow et al., NUCLEIC ACIDS RES. 37:D983 (2009) and PLACE (Higo et al., NUCLEIC ACIDS RES. 27:297 (1999) databases using STAMP software (Mahony and Benos, NUCLEIC ACIDS RES. 35:W253 (2007). Only motifs that were preferentially located at one or more common positions within the promoters of each expression cluster were reported.

### Results

The expression patterns of 65 probe sets, representing genes associated with nitrogen transport and assimilation, were analyzed in various maize tissues at different stages of plant development. Interestingly, the highest expression of nitrogen-related genes in reproductive tissues was in anthers.

Tissue and/or Developmental Stage-Selective Expression.

65 probes, representing nitrogen-related genes, were determined to be differentially expressed in vegetative tissues during particular stages of plant development. Four gene expression clusters were identified.

Cluster 1:

Cluster one probes showed juvenile-selective expression, with peak expression in juvenile (Ve-V2) roots and vegetative stage (Ve-V5) leaves. Probe sets in Cluster 1 corresponded to several interesting categories of genes, including nitrate transporters (NRT1.1, NRT2.1) and ammonium transporters (Osamt1.1, Osamt1.3, Osamt2.2, Osamt3.1, Osamt5.1).

Cluster 2:

Cluster 2 probes showed leaf-selective expression, with peak expression in vegetative stage (Ve-V5) leaves. Probe sets in Cluster 2 corresponded to several interesting categories of genes, including nitrate transporters (NRT1.5, Atntl1), nitrate reductases (NR1, NR2) and a low-affinity nitrite transporter (NiTR1) that had not previously been shown in maize (Sugiura et al., PLANT CELL PHYSIOL. 48:1022 (2007)).

Cluster 3:

Cluster 3 probes showed root-selective expression and were expressed throughout plant development (Ve-24DAP). Probe sets in Cluster 3 corresponded to several interesting categories of genes, including glutamine synthases (Gln4/Gln1-4, Gln5.Gln1-5) and glutamate synthases. Also included in Cluster 3 were probe sets corresponding to ammonium transporters (Osamt1.1, Osamt5.2), the low-affinity nitrite transporter NRT1.1 and the companion protein of the high affinity nitrate transporter complex (NAR2.1).

Cluster 4:

Cluster 4 probes showed leaf-selective expression, with peak expression in older (V5-31DAP) leaves. Probe sets in Cluster 4 corresponded to several interesting categories of genes, including nitrate reductase (NR1) and nitrate transporters (Atntl1), ammonium transporters (Osamt2.1, Osamt2.3, Osamt3.3), as well as one NAR2 paralog and the low-affinity nitrite transporter NiTR1.

Over-Represented Cis-Acting Promoter Motifs.

Several over-represented cis-acting motifs were identified in the promoter regions of the nitrogen-related genes corresponding to probe sets in Clusters 1-4 (Table 2).

Cluster 1:

Several over-represented cis-acting motifs were identified in the promoter regions of the nitrogen-related genes expressed in Cluster 1. As shown in Table 2, three of those motifs are similar to previously identified promoter elements.

As shown in Table 2, one of the over-represented cis-acting promoter motifs identified in Cluster 1, CGACCNTT (SEQ ID NO:1), resembles the core of the pseudo-palindromic nitrogen response element (NRE) that was recently identified in dicot nitrite reductase (NiR) genes as being necessary and sufficient for nitrate-responsive gene transcription (Konishi and Yanagisawa, PLANT J. 63:269 (2010)). Within Cluster 1, the CGACCCTT motifs (SEQ ID NO:10) was over-represented in the promoters of genes encoding nitrate transporters (Nrt1, Nrt2. 1) and ammonium transporters (Table 3).

TABLE 3
NRE/Acore motifs identified in the promoter regions of nitrogen-related
genes corresponding to probe sets in Cluster 1.

| Maize | | | | | | Identity |
|---|---|---|---|---|---|---|
| annotation | Description | NRE/ACore motif | | | Downstream nucleotides | percentage |
| GRMZM2G | high affinity nitrate | −319 | TGATCCT | −313 | GGCTGATCCCACGGGATGAGGC | 93.24% |
| 010280 | transporter | | T | | CAAGCCCA | |
| | (nrt2.1) | −53 | CGACCCT | −47 | CATGTCCATGACACGCCAGAGC | 100.00% |
| | | | T | | TCAATCTT | |
| GRMZM2G | nitrate and chloride | −424 | CGACCTT | −418 | ATGATTTTGGGTCTTCTTTTTG | 96.65% |
| 453320 | transporter | | T | | AAAACGAA | |
| GRMZM2G | ammonium | −292 | CGACCCT | −286 | TTGCCGTGCGCTGCTCGAGTCT | 100.00% |
| 080045 | transporter | | T | | GCCTAACC | |
| | | −223 | CGTGCCT | −217 | CCGTTTTAGGTTTGATTCGTCG | 90.03% |
| | | | T | | ACTTGAAT | |
| | | −95 | CGACCAT | −89 | TTCTTGTCATCTCTTCAGAACA | 91.12% |
| | | | G | | GTCTGAAG | |
| GRMZM2G | symbiotic ammonium | −493 | CCACCGT | −487 | AGATCGGTCACCAGGTCATAGT | 91.12% |
| 082343 | transporter | | T | | CCACCATG | |
| | | −303 | CGATCGT | −297 | CGTCGGGCGATTGTTTATCCCC | 95.61% |
| | | | T | | GGACTAAA | |
| GRMZM2G | ammonium transporter | −208 | CGACACT | −202 | TATTGTAATTTTGGACTAGTCT | 94.29% |
| 028736 | (Osamt1.3) | | T | | CTCTTTTT | |
| | | −146 | CGATCTT | −140 | CTACAGTGCAAGATAATAATGG | 95.61% |
| | | | T | | AGTATCTC | |
| GRMZM2G | ammonium transporter | −169 | CGGCCCT | −163 | GGATCCTGGCCACCGTGGGTGG | 90.67% |
| 175140 | (Osamt1.1) | | G | | GCAGATTC | |
| | | −111 | CGATCCG | −105 | TGTTTGTTTTGCCGAATCAAAA | 93.24% |
| | | | T | | CTGCAATT | |
| GRMZM2G | ammonium transporter | −160 | CGATCCT | −154 | CTCTTCTCTCCTAGAGCCACTC | 98.95% |
| 335218 | (Osamt2.2; Osamt3.1; | | T | | ACCGGCGC | |
| | Osamt5.1) | | | | | |
| | maize transporter | | CGACCCT | | | |
| | consensus | | T | | | |
| | dicotyledon nitrate | | tGACcCT | | | |
| | reductase (Konishi | | T | | | |
| | et Yanagisawa, 2010) | | | | | |

Also as shown in Table 2, one of the over-expressed cis-acting promoter motifs identified in Cluster 1, CCACGTGC (SEQ ID NO:2), resembles the reverse complement of the G-box 10 element, which was first characterized in tobacco (Nicotianatabacum) and has been shown to confer near-constitutive gene expression in roots, leaves, seeds and some reproductive tissues (Ishige et al., PLANT J. 18:443 (1999)). The CCACGTGC motif also resembles the E-Box (CANNTG), which has been shown to be critical for nitrate induction of the Nia1 (nitrate reductase) promoter in Arabidopsis (Wang et al., PLANT PHYSIOL. 154:423 (2010)).

Also as shown in Table 2, one of the over-expressed cis-acting promoter motifs identified in Cluster 1, NNANGCSGCW (SEQ ID NO:4), resembles the Negative Regulator of Glucose-repressed Genes 1 (NRG1), which was upstream of genes involved in regulating cell sugar status in yeast (Park et al., MOL. CELL. BIOL. 19:2044 (1999); Zhou and Winston, BMC GENETICS 2:5 (2001)).

Cluster 2:

Several over-represented cis-acting motifs were identified in the promoter regions of the nitrogen-related genes expressed in Cluster 2. As shown in Table 2, two of those motifs are similar to previously identified promoter elements.

As shown in Table 2, one of the over-represented cis-acting promoter motifs identified in Cluster 2, AGTCGG (SEQ ID NO:5), resembles a low temperature responsive element (LTRE), also called C/DRE, that mediates ABA-independent response to cold, amplified by light viaphytochrome signalling (Kim et al., J. BIOL. CHEM. 277:38781 (2002)).

Also as shown in Table 2, one of the over-represented cis-acting promoter motifs identified in Cluster 2, TAGYCRGC (SEQ ID NO:6), resembles the YAP1 element, which was first identified as the binding site of the yeast YAP1 protein, a conserved protein across eukaryotes including Arabidopsis (Babiyuchuk et al., J. BIOL. CHEM. 270:26624 (1995)). YAP1 has been shown to regulate the yeast cell cycle in response to oxidative stress, but also able to regulate cell elongation (Carmel-Harel et al., MOL. MICROBIOL. 39:595 (2001); Moye-Rowley et al., GENES DEV. 3:283 (1989); Yokoyama et al., EMBO REP. 7:519 (2006)).

Cluster 3:

Several over-represented cis-acting motifs were identified in the promoter regions of the nitrogen-related genes expressed in Cluster 3. As shown in Table 2, one of those motifs is similar to at least one previously identified promoter element.

As shown in Table 2, one of the over-represented cis-acting promoter motifs identified in Cluster 3, TRTCCGTACG (SEQ ID NO:7), resembles the binding site of the Forkhead (FHL1) transcription factor in yeast, a repressor of ribosome-encoding genes during glucose starvation (Hermann-Le Denmat et al., MOL. CELL. BIOL. 14:2905 (1994)).

Cluster 4:

Several over-represented cis-acting motifs were identified in the promoter regions of the nitrogen-related genes expressed in Cluster 4. As shown in Table 2, one of those motifs (NANGAG; SEQ ID NO:8) is a previously unidentified promoter element.

### DISCUSSION

### Global Analysis of Nitrogen-Related Genes During Maize Development

Recent transcriptome studies have highlighted global gene expression patterns in maize, examining juvenile leaves over a 24 hour period (Jońezyk et al., PLoS ONE 6:e23628 (2011); a maturation gradient along a juvenile leaf (Li et al., NAT. GENETICS 42:1060 (2010)); leaves during the transition from juvenile to adult vegetative stages (Strable et al., PLANT J. 56:1045 (2008)); and different organs across development with a focus on lignin biosynthetic genes (Sekhon et al., PLANT J. 66:553 (2011)). The focus of this study was to examine the expression of nitrogen transporter and assimilation genes across maize development, particularly in roots and leaves. Four vegetative gene expression clusters were identified, specifically an early stage-selective cluster (Cluster 1), an early leaf-selective cluster (Cluster 2), a root-selective cluster (Cluster 3) and a late leaf-selective cluster (Cluster 4). This clustering pattern demonstrates that distinct subsets of nitrogen-related paralogs are preferentially expressed in leaves compared to roots in maize. This data is consistent with recent data from Arabidopsis showing differences in the expression of nitrogen-related genes in young roots versus young leaves during nitrogen starvation (Krapp et al., PLANT PHYSIOL. 157:1255 (2011)).

Second, our data demonstrates that distinct subsets of nitrogen-related genes are selectively expressed at different developmental phases of the shoot in maize (Poethig, PLANT PHYSIOL. 154:541 (2010); Poethig, SCIENCE 250:923 (1990)). For example, the shift from vegetative to reproductive development altered the expression of nitrogen-related genes in leaves, consistent with changes in nitrogen demand during grain fill (Peng et al., FIELD CROPS RES. 115:85 (2010)).

It is noteworthy that the expression of Cluster 1 nitrogen-related genes in maize roots was particularly selective for juvenile stages of development. This latter result suggests that shoot-based phases of development (Poethig, SCIENCE 250:923 (1990)), for instance juvenile versus adult, may have biological impacts on the root transcriptome, a finding that warrants further investigation. Previous studies have demonstrated that root growth is inhibited by accumulation of nitrate in the shoot (Scheible et al., PLANT J. 11:671 (1997)) and that nitrogen stress responses are highly coordinated between the shoot and root systems of maize (Gaudin et al., CROP SCI. 51:2780 (2011); Gaudin et al., PLANT CELL ENVIRON. 34:2122 (2011); Peng et al., FIELD CROPS RES. 115:85 (2010)).

A particularly interesting finding from this study was the high level of gene expression of many nitrogen-related probe sets in anthers. In fact, >14/65 probe sets showed peak expression in anthers (VT stage), including probe sets that showed root or shoot-selective expression during vegetative development. Given that the normalization method used, named RMA (Irizarry et al., NUCLEIC ACIDS. RES, 31:e15 (2003)), standardizes the gene expression ranges between arrays (e.g. relative gene expression from anther RNA has the same range than leaf RNA after normalization), this result does not appear to be an artefact.

Promoter Motifs Underlying Stage-Selective Gene Expression.

A proximal 43 bp nitrogen response element (NRE) was recently identified in the promoters of Arabidopsis nitrite reductase (NiR) genes and shown to be both necessary and sufficient for nitrate-induced gene expression and independent of repression by glutamine (Konishi et al., PLANT J. 63:269 (2010)). An alignment of dicot NiR promoters revealed a pseudo-palindrome bipartite consensus motif (5′-(t/c)GACcCTTN₁₀AAG(a/g)-3 in the promoter region at approximately −100 bp to −240 bp (Konishi et al., PLANT J. 63:269 (2010)).

Here we show that a conserved 8 bp motif within this region is also conserved upstream of maize genes (consensus: CGACCCTT) encoding nitrate and ammonium transporters (Table 3). The motif is preferentially located in the −200 to −250 bp region of the corresponding promoters (Tables 2, 3). We therefore suggest that this 8 bp motif defines an important cis-acting element of vegetatively expressed nitrogen-related genes in angiosperms. We propose that this conserved 8 bp motif be referred to as the NRE/Acore motif (NRE-43 angiosperm core motif).

Prior to the discovery of the proximal NRE motif noted above (Konishi et al., Plant J. 63:269 (2010)), a distal NRE [core A(c/g)TCA] was shown to be conserved in the promoters of nitrate reductase (NR) and NiR genes in both dicots and monocots including maize, but was not tested for sufficiency of nitrate induction (Hwang et al., PLANT MOL. BIOL. 36:331 (1998)). In maize, several of the distal NRE [A(g/c)TCA] were located in the −500 to −1000 bp promoter region (Hwang et al., PLANT MOL. BIOL. 36:331 (1998)), more distal than the region analyzed in this study, perhaps explaining why we did not recover this motif in our search.

Our analysis revealed another nitrogen-related motif of interest, the E-box motif (CANNTG) forming the core of the G-box 10 element (Table 2). The E-box motif overlaps the HVH21 motif which was shown to be important for nitrate induction of the Arabidopsis Nia1 promoter (Wang et al., PLANT PHYSIOL. 154:423 (2010)). Both the E-box and the G-box are related and bind bHLH transcription factors, of which >147 members have been identified in Arabidopsis (Toledo-Ortiz et al., PLANT CELL ONLINE 15:1749 (2003)).

In addition to these nitrogen-associated motifs, two cis-acting elements related to carbon availability were identified in our study, NRG1 and FHL1 (Table 2). The NRG1 motif represents the binding site of the yeast NRG1 protein, a repressor of sucrose synthase (SUC2), glucose invertase, galactokinase 1, 4 and genes involved in gluconeogenesis and the Krebs Cycle (Park et al., MOL. CELL. BIOL. 19:2044 (1999); Zhou and Winston, BMC GENETICS 2:5 (2001)). This glucose-repression pathway is conserved between yeast and plants (Jang and Sheen, TRENDS PLANT SCI. 2:208 (1997); Ramon et al., THE ARABIDOPSIS BOOK at 1 (2008)). Increased sugar levels in roots have been shown to activate the expression of the nitrate transporter genes Nrt2.1 and Nrt1.1 in Arabidopsis (Lej ay et al., PLANT J. 18:509 (1999)). In Cluster 1, the NRG1-like motif was similarly found in the promoters of Nrt1.1 and Nrt2.1. As Cluster 1 genes were more expressed early in development, when the maize seedling is heterotrophic and dependent on the grain reserve, one possibility is that the NRG1 motif helps to regulate nitrogen uptake based on the availability of sugar from the grain. Carbon is needed for the energy required for nitrogen uptake/assimilation and to provide carbon skeletons for amino acid synthesis (Masclaux-Daubresse et al., ANNALS BOTANY 105:1141 (2010)).

The second carbon-related motif was FHL1 (Table 2), corresponding to the binding site of the FHL1 protein, a repressor of genes important for ribosome biogenesis during glucose starvation in yeast (Hermann-Le Denmat et al., MOL. CELL. BIOL. 14:2905 (1994); Kim et al., J. BIOL. CHEM. 277:38781 (2002)). The tobacco ortholog of FHL1 is functional in yeast demonstrating the conservation of this protein across eukaryotes (Kim et al., J. BIOL. CHEM. 277:38781 (2002)). FHL1 is regulated by the Target of Rapamycin (TOR) pathway identified in both yeast and plants, and shown to play a central role in turning protein translation capacity to nutrient availability in the former during growing tissues (Diaz-Troya et al., AUTOPHAGY 4:851 (2008); Dobrenel et al., BIOCHEM. SOC. TRANSACTIONS 39:477 (2011)). The FHL1 motif was over-represented in genes encoding nitrate and ammonium transporters, nitrate reductase and glutamine synthases in Cluster 3, the root-selective expression cluster. It may be that the FHL1/TOR regulon plays an important role in coordinating carbon availability and nitrogen uptake and assimilation with the protein synthesis machinery in maize roots.

Nitrite Transporter Expression.

We report the expression of probe sets corresponding to putative maize ortholog(s) of a nitrite transporter (NiTR), a gene class not previously been reported in maize. A gene encoding NiTR (Nar1) was initially reported in plants in Chlamydomonas chloroplasts (Rexach et al., PLANT CELL ONLINE 12:1441 (2000)). In higher plants, an NiTR gene was first reported in cucumber (CsNitr1-L), along with a functional ortholog tested in Arabidopsis (At1g68570) where a knockout mutation showed a five-fold increase in nitrite accumulation in leaves (Sugiura et al., PLANT CELL PHYSIOL. 48:1022 (2007)). The cucumber NiTR protein was localized to the inner envelope membrane of chloroplasts, where it was hypothesized to load nitrite from the cytoplasm into the stroma of the chloroplast during nitrate assimilation (Sugiura et al., PLANT CELL PHYSIOL. 48:1022 (2007)). Consistent with chloroplast localization, transcripts of the putative maize NiTR(s) orthologs were detected in the two leaf-selective expression clusters (Clusters 2 and 4) with strong expression in the husk leaves surrounding the cob, but not in the root-selective clusters.

### Example 2

### Tissue- and/or Stage-Specific Gene Expression in Maize

Microarray analysis was used to identify gene expression clusters in various maize tissues at different stages of plant development and then to identify over-represented cis-acting promoter motifs corresponding to each gene expression cluster. Finally, organ- and/or stage-specific genes that may serve as developmental markers for stages within juvenile maize development were identified.

### Materials and Methods

Plant Growth and Tissue Harvest.

Proprietary hybrid seeds (SRG150, Syngenta Biotechnology, Inc., Durham, N.C.) were grown in a greenhouse during the summer of 2007 at the University of Guelph in Ontario, Canada. Plants were grown semi-hydroponically in pots containing Turface® clay soil conditioner (PROFILE Products, LLC, Buffalo Grove, Ill.) at 50% relative humidity with 16 hours of light (about 600 μmol m⁻² s⁻¹) at 28° C. and eight hours of dark at 23° C. Plants were watered with a nutrient solution containing: 0.4 g/L 28-14-14 fertilizer, 0.4 g/L 15-15-30 fertilizer, 0.2 g/L NH₄NO₃, 0.4 g/L of MgSO4.7H₂O and 0.03 g/L of micronutrient mix (S, Co, Cu, Fe, Mn, Mo and Zn). Three biological replicates per tissue/stage were harvested, always at about 11:00 am.

Microarray Analysis and Normalization.

Total RNA was isolated using Trizol reagent and RNeasy column to purify the extracts from protein and perform a DNAse treatment (Dallas et al, 2005; BMC Genomics. 2005; 6: 59; doi: 10.1186/1471-2164-6-59; Bi et al. BMC Genomics 8:281(2007)). Messenger RNA (mRNA) was isolated from 50 maize tissues at different stages of plant development, ranging from seedling emergence (Ve) to 31 days after pollination (31DAP) (three biological replicates per tissue/stage) (Table 1). mRNA was hybridized onto 150 customized maize Affymetrix 82K Unigene arrays (Affymetrix, Inc., Santa Clara, Calif.) as described by Wagner and Radelof, J. BIOTECH. 129:628 (2007). Gene expression was normalized using the RMA method from Bioconductor as described by Gentleman et al., GENOME BIOL. 5:R80 (2004), and Irizarry et al., NUCLEIC ACIDS RES. 31:e15 (2003).

Identification of Gene Expression Clusters.

Gene expression clusters were identified using K-means clustering, as described by Hartigan and Wong, J. ROYAL STAT. SOC. SERIES C (APPLIED STAT.) 28:100 (1979).

Statistical Analysis of Tissue-Specific Gene Expression.

Tissue-specific and tissue-selective gene expression was analyzed using the Intersection Union Test (IUT; Berger and Hsu, STAT. SCI. 11:283 (1996)) using R coding modified from BayesianlUT (Katholieke Universiteit Leuven, Leuven, Netherlands; available at ppw.kuleuven.be/okp/software/BayesianlUT/; Van Deun et al., BIOINFORMATICS 25:2588 (2009)). The null hypothesis tested by IUT was that expression of a given gene in the target tissue was not significantly different from expression of that gene in one or more other tissues. The alternate hypothesis tested by IUT was that expression of a given gene was higher in the target tissue than in any other tissue. A gene was determined to be tissue-specific if all possible pairwise t-tests between the target tissue and the other tissues analyzed indicated a significant difference in mean gene expression. Sidak's adjustment (Sidak, J. AMER. STAT. ASSOC, 62:626 (1967)), which is equivalent to the Bonferroni correction, was used to correct the multiple testing methodology of the custom R package.

Statistical Analysis of Differential Gene Expression.

Differential gene expression was measured by fitting linear models to the data using the Limma Package from Bioconductor, as described by Smyth in Gentleman et al., BIOINFORMATICS AND COMPUTATIONAL BIOLOGY SOLUTIONS USING R AND BIOCONDUCTOR (2005). The linear models were adjusted using the empirical Bayesian method (modified t-test) form the Limma Package and were corrected for multiple testing as described by Benjamini and Hochberg, J. ROYAL STAT, SOC. SERIES B 57:289 (1995). The significance threshold of the adjusted p-value was set at 0.05.

Gene Ontology Detection.

To link the microarray probe sets to known genes, protein sequences corresponding to the probes were retrieved from the Genbank® database using a customized Peri script and then matched to the filtered B73 maize genome (release 4a.53, MaizeSequence.org; Schnable et al., SCIENCE 326:1112 (2009)). A list of MaizeSequence.org gene IDs was created for each tissue comparison, and a gene ontology (GO) term was assigned to each retrieved gene using agriGO (Du et al., NUCLEIC ACIDS RES. 38:W64 (2010)). The agriGO hypergeometric test was used to identify GO terms that were enriched for a specific tissue/stage.

Promoter Motif Discovery.

The promoter region (−500 to +1, relative to the ATG start codon) of each of the genes corresponding to a tissue-specific or tissue-selective array probe was analyzed for over-represented de novo cis-acting motifs (≧6 bp) using Promzea (available at promzea.org), a Perl-based motif discovery program that filters and combines the results of three motif discovery tools (Weeder (Pavesi et al., BMC BIOINFORMATICS 8:46 (2007)), MEME (Bailey et al., PROC. SECOND INTL. CONF. INTELLIGENT SYS, MOL. BIOL. 28-36 (1994)) and BioProspector (Liu et al., PAC, SYMP. BIOCOMPUTING 2001:127 (2001))) and statistically validates identified motifs using the hypergeometric test or the binomial test. All significant motifs identified in the aforementioned search were compared to previously defined motifs in the Athamap (Bulow et al., NUCLEIC ACIDS RES. 37:D983 (2009) and PLACE (Higo et al., NUCLEIC ACIDS RES. 27:297 (1999) databases using STAMP software (Mahony and Benos, NUCLEIC ACIDS RES. 35:W253 (2007). Only motifs that were preferentially located at one or more common positions within the promoters of each expression cluster were reported.

### Results

Combining results from all tissues and stages, 53,554 probe sets were expressed (relative expression >100), representing 64.8% of the array. 6,955 (13%) of the expressed probe sets were selectively-expressed in tissues from juvenile plants (i.e., plants in the Ve, V1 and V2 stages of plant development). Interestingly, there were 145 probe sets that were selective for a single-stage juvenile root or leaf tissue, identifying these as developmental markers.

Ninety-five percent (51,156) of the expressed probe sets were expressed in above-ground tissues (stalk, leaves, etc.). Of those, 41,948 (82%) of the expressed probe sets were expressed in leaves; 77.3% of the expressed probe sets were detected leaves from juvenile plants and 74.3% were detected in leaves from older plants. Four thousand three hundred and thirty three (8.1%) of the expressed probe sets were selectively-expressed in leaves from juvenile plants.

41,450 (77.3%) of the expressed probe sets were expressed in below-ground tissues (seminal/nodal roots); 72.2% of the expressed probe sets were expressed in roots from juvenile plants and 74.1% were detected in roots from older plants. 1,785 (3.3%) of the expressed probe sets were selectively-expressed in roots from juvenile plants. 2,767 (5.1%) of the expressed probe sets were selectively-expressed in roots from older plants.

### Stage-Selective Leaf Expression.

Ve Coleoptile-Selective Expression:

43 probe sets, representing 23 genes, showed coleoptile-selective expression at the Ve stage of plant development. Several Ve coleoptile-selective genes of interest are shown in Table 4, including a gene that encodes indole-3-glycerol-phosphate synthase (IGPS) and a gene that encodes indole-3-acetic acid (IAA). IAA is an important enzyme in the precursor pathway for synthesis of auxin (Ouyang et al., PLANT J. 24:327 (2004)) and has previously been shown to be produced in the coleoptiles tip of maize plants (Mori et al., PLANT SCI, 168:467 (2005)). IGPS catalyzes the fourth step in tryptophan biosynthesis (Ouyang et al., PLANT J. 24:327 (2004)) and is also involved in the synthesis of protective secondary metabolites, including DIMBOA (2,4-dihydroxy-7-methoxy-1,4-benzoxazin-3-one, KEGG: zma00402), an important anti-pathogen compound produced by grasses (Frey et al., PHYTOCHEMISTRY 70:1645 (2009)). Consistent with these findings, genes encoding three enzymes in the DIMBOA biosynthetic pathway also showed Ve coleoptile-selective expression: Benzoxazinless1, Benzoxazinone Synthesis 2 and Benzoxazinone Synthesis 8. Frey et al., PHYTOCHEMISTRY 70:1645 (2009). Interestingly, Cambier et al., PHYTOCHEMISTRY 53:223 (2000), have shown that DIMBOA is abundant in maize seeds and seedlings up to 10 days after germination, consistent with our gene expression data.

TABLE 4
Exemplary leaf/stage-specific genes

| Gene name | Description |
|---|---|
| Ve Leaf | |
| GRMZM2G169516 | Putative indole-3-glycerol-phosphate synthase, |
| | hypothetical protein LOC100274566 |
| GRMZM2G085381 | Benzoxazineless 1, Indole-3-glycerol phosphate |
| | lyase, chloroplastic Precursor (EC 4.1.2.8) |
| GRMZM2G085661 | Benzoxazinone synthesis 2, benzoxazineless 2, |
| | Cytochrome P450 71C4 |
| GRMZM2G085054 | Benzoxazinone synthesis8 (bx8), UDP- |
| | glucosyltransferase |
| V2 Leaf | |
| GRMZM2G099367 | Ortholog of Arabidopsis chloroplast |
| | ACCLIMATION OF PHOTOSYNTHESIS |
| | TO ENVIRONMENT (APE1) |
| GRMZM2G477236 | lil3 protein |
| GRMZM2G007160 | chloroplastic hydrolase, alpha beta fold |
| | family protein |
| GRMZM2G012717 | light responsive zinc finger (B-box type) |
| | family protein |
| GRMZM2G034243 | chloroplast NAD(P)H dehydrogenase complex, |
| | NDH-DEPENDENT CYCLIC ELECTRON FLOW |
| | 1 (NDF2) |
| GRMZM2G038494 | chloroplastic GTP-binding protein-related |
| GRMZM2G066107 | chloroplast CAAX amino terminal protease |
| | family protein |
| GRMZM2G067853 | chloroplast ATPase cadmium-transporting |
| GRMZM2G074857 | EMB1374, chloroplast sulfur E |
| GRMZM2G105539 | anion-transporting ATPase family protein |
| | located in chloroplast |
| GRMZM2G111216 | CHLOROPLAST STEM-LOOP BINDING |
| | PROTEIN 41 |
| GRMZM2G319109 | FZO-LIKE (FZL) GTP binding GTPase thiamin- |
| | phosphate diphosphorylase regulation of |
| | organization of the thylakoid network |

Several over-represented cis-acting motifs were identified in the promoters of Ve coleoptile-selective genes. Two of those motifs, which we refer to as Maize Ve-stage Leaf (MVeL) motifs include MVeL1 (SEQ ID NO:21) and MVeL2 (SEQ ID NO:22) are similar to previously identified cis-acting regulatory elements RE-alpha and RE-beta, respectively. Degenhardt et al., PLANT CELL ONLINE 8:31 (1996). The RE motifs were first characterized as adjacent elements in the promoter of the Lemnagibbalight-harvesting complex II gene Lhcb2.1 and were shown to be responsible for phytochrome-mediated regulation, facilitating promoter repression in darkness. Id.

V1 Leaf-Selective Expression:

15 probe sets, representing 5 genes, showed leaf-selective expression at the V1 stage of plant development. Given the number of genes in the gene expression cluster, no over-represented cis-acting motifs were identified in the promoters of V1 leaf-selective genes.

V2 Leaf-Selective Expression:

59 probe sets, representing 29 genes, showed leaf-selective expression at the V2 stage of plant development. Ten of the 29 V2 leaf-selective genes are associated with chloroplasts. Several V2 leaf-selective genes of interest are shown in Table 4, including an ortholog of the Arabidopsis Acclimation of photosynthesis to environment (Ape1) gene and a gene encoding the Light-Harvesting Like 3 (LIL3) protein. Ape1 mutants have been shown to reduce the ability of a plant to acclimate following a shift from low light to high light and are unable to restore rates of photosynthesis and/or levels of chlorophyll (Walters et al., PLANT PHYS. 131:472 (2003)). LIL3 appears to be the essential protein that binds to chlorophyll during chloroplast biogenesis to enable formation of the light harvesting complex (LHC) following a shift from extended darkness to light (de-etiolation) (Reisinger et al., FEBs LETTERS 582:1547 (2008)). These data suggest the existence of light-regulated gene expression specific to V2 leaves in maize.

Several over-represented cis-acting motifs were identified in the promoters of V2 leaf-selective genes. One of those motifs, referred to as Maize V2-stage Leaf (MV2L) motifs, is MV2L1 (SEQ ID NO:23) which is similar to a previously identified ABA-regulated, cis-acting ABRE3 element found in the barley Hva22 gene. Shen and Ho, PLANT CELL ONLINE 7:295 (1995). MV2L1 also contains an ACGT A core previously identified in the A-box subfamily of ACGT elements, which have been shown to bind bZIP transcription factors (Foster et al., FASEB J. 8:192 (1994)) and a perfect 5 bp match to SORLIP1, a motif that is over-represented in phytochromeA-induced promoters (Hudson et al., PLANT PHYSIOL. 133:1605 (2003)).

### Stage-Selective Root Expression.

Ve Root-Selective Expression:

20 probe sets, representing 11 genes, showed root-selective expression at the Ve stage of plant development. Several over-represented cis-acting motifs were identified in the promoters of Ve root-selective genes. One of these motifs, referred to as Maize Ve-stage Root (MVeR) motifs, is MVeR1 (SEQ ID NO:24) is similar to the GATGG core of the in vivo HOX (homeodomain) binding site of the PBC-HOX1/LAB heterodimer complex [TGAT(t/g)GAT(t/g)] from Drosophila, which is required to specify anteroposterior segment identity in multicellular animals (Ebner et al., DEV. 132:1591 (2005); Mann et al., CURRENT TOPICS IN DEVELOPMENTAL BIOLOGY 63-101 (2009)).

V1 ROOT-SELECTIVE EXPRESSION:

17 probe sets, representing 10 genes, showed root-selective expression at the V1 stage of plant development. Several V1 root-selective genes of interest are shown in Table 5, including a homolouge of AtNtr1.1, which encodes a high-affinity nitrogen transporter (Crawford and Glass, TRENDS PLANT SCI. 3:389 (1998); Gaugin et al., PLANT CELL ENVRION. 34:2122 (2011)), and a gene encoding zeaxanthinepoxidase (ZEP). ZEP, trace elements of which have previously been found in roots, converts zeazanthin to violaxanthin (Howitt and Pogson, PLANT CELL ENVIRON. 29:435 (2006)) and catalyzes the first step of abscisic acid (ABA) biosynthesis. Given its importance in ABA biosynthesis, ZEP loci are often referred to as Aba1 and Aba2. These data are suggestive of stage- and tissue-specific regulation of nitrogen uptake and stress responses, consistent with previous studies carried out in Arabidopsis and tobacco (Audran et al., PLANT PHYSIOL. 118:1021 (1998); Krapp et al., PLANT PHYSIOL. 157:1255 (2011)).

TABLE 5
Exemplary root/stage-specific genes

| Gene name | Description |
|---|---|
| V1 Root (seminal) | |
| GRMZM2G086496 | Ortholog of Arabidopsis NRT1.1, CHL1 |
| GRMZM2G127139 | zeaxanthinepoxidase, chloroplast precursor |
| V2 Root (seminal) | |
| GRMZM2G072529 | ACC oxidase |
| GRMZM2G166639 | ACC oxidase |
| GRMZM2G114503 | RADIALIS, ortholog of ATRL6 |
| GRMZM2G028736 | ammonium transmembrane transporter, ortholog of |
| | ATAMT1; 2 |
| GRMZM2G070087 | Rice protein inorganic phosphate transporter 1-7, |
| | putative |

Several over-represented cis-acting motifs were identified in the promoters of V1 root-selective genes. One of these motifs, referred to as Maize V1-stage Root (MV1R) motifs, is MV1R1 (SEQ ID NO:25), which is similar to the C1gR binding site (GTTCGC GCG) in Streptomyces bacteria, and which regulates the transcription of genes involved in an unusual form of prokaryotic differentiation (Bellier and Mazodier, J. BACTERIOLOGY 186:3238 (2004)).

V2 seminal root-selective expression:

17 probe sets, representing 19 genes, showed seminal root-selective expression at the V2 stage of plant development. Several V2 seminal root-selective genes of interest are shown in Table 5, including genes encoding two distinct aminocyclopropanecarboxylate (ACC) oxidases, the Radialis (RAD) gene, a putative homologue of a rice phosphatase transporter and a homologue of an Arabidopsis ammonium transporter. ACC oxidases catalyze the last step in the biosynthesis of ethylene, a hormone that has been shown to play a critical role during root development (Ruzicka et al., PLANT CELL ONLINE 19:2197 (2007). At higher concentrations, ethylene represses root cell elongation via auxin (Id.) and can inhibit the growth of immature primary and lateral roots (Id.; Gallie et al., PLANT MOLEC. BIOL. 69:195 (2009); Tian et al., NEW PHYTOLOGIST 184:918 (2009)). At low concentrations, ethylene has been shown to promote lateral root initiation and enlargement (Ivanchenko et al., PLANT J. 55:335 (2008)). Thus, V2 seminal root-specific expression of one or more ACC oxidases may help to regulate the change in root architecture observed between juvenile and adult maize stages (Singh et al., PLANT SOIL 333:287 (2010). The maize Rad gene is a putative homologue of AtATRL6/RSM3, which encodes a MYB transcription factor first identified in Antirrhinum as regulating floral asymmetry and shown to be expressed along ab-adaxial or radial domains (Baxter et al., PLANT J. 52:105 (2007)). Interestingly, in Arabidopsis, a mutation in a paralog of ATRL6, RSM1 has been shown to participate in an important ethylene-auxin cross-talk signalling pathway involving HOOKLESS 1 (Hamaguchi et al., BIOSCI. BIOTECH. BIOCHEM. 72:2687 (2008)).

Several over-represented cis-acting motifs were identified in the promoters of V2 seminal root-selective genes. One of these motifs, referred to as Maize V2-stage Root (MV2R) motifs, is MV2R1 (SEQ ID NO:26), which is similar to the cis-acting plant sugar response element (SURE box, consensus (a/t)(t/c)ACT(a/g)T(t/g)) (Grierson et al., PLANT J. 5:815 (1994)). The SURE box was first identified as the SP8b motif (TACTATT) in the sugary roots of sweet potatoes (Ishiguro et al., MOL. GEN. GENETICS 244:563 (1994)), which binds the WRKY-family SP8 transcription factor in the sugary roots of sweet potatoes (Agarwal et al., MOL. BIOL. REP. 38:3883 (2011))³⁴³⁵. Subsequently, it has been demonstrated that a few WRKY transcription factors, such as SP8, may independently bind to SURE as well as a canonical W-box element (Agarwal et al., MOL. BIOL. REP. 38:3883 (2011); Rushton et al., TRENDS PLANT SCI. 15:247 (2010)). Included on this list of WRKY proteins is the transcription factor SUSIBA2/HvWRKY46, which regulates expression of anabolic carbohydrate enzymes in barley endosperm (Sun et al., PLANT CELL ONLINE 15:2076 (2003)). These data suggest that a SURE-WRKY regulon may operate in V2 stage maize roots to modulate gene expression based on sugar availability.

V2 Nodal Root-Selective Expression:

No probe sets showed selective expression for V2 nodal roots.

### Differential Gene Expression in Seminal and Nodal Roots.

Microarray analysis was used to further characterize the differences in gene expression between seminal roots and nodal roots in young maize plants. To reduce the effects of stage-specific expression differences (see discussion above), results were pooled from the V2 and V5 stages of plant development.

A total of 1035 probe sets were differentially expressed between the two root organ types: 711 probe sets were up-regulated in seminal roots as compared to nodal roots and 334 probe sets were up-regulated in nodal roots as compared to seminal roots. In this case, the differentially expressed probe sets represented various types of genes. For example, probe sets corresponding to at least one gene associated with heme binding were selectively up-regulated in each of the two root organ types. Both root organ types likewise showed selective up-regulation of probe sets corresponding to at least one gene associated with iron ion binding and probe corresponding to at least one gene associated with electron carrier activity.

Probe sets corresponding to several interesting categories of genes were selectively up-regulated in nodal roots. For instance, probe sets corresponding to genes associated with antioxidant activity and oxidative stress responses were up-regulated in nodal roots, suggesting that the differential expression of such genes in nodal roots may play a role in the hormonal control of root development (De Tullio et al., PLANT PHYSIOL. BIOCHEM. 48:328 (2010)). Likewise, probe sets corresponding to genes associated with sexual reproduction were also up-regulated in nodal roots but not in seminal roots.

Probe sets corresponding to genes associated with chloroplasts and photosynthesis, including the thylakoid membrane, were selectively up-regulated in seminal roots. Somewhat surprisingly, probe sets corresponding to 7 out of the 21 nuclear genes associated with Photosystem I were also selectively up-regulated in seminal roots. Previous studies have shown that Photosystem I genes can be expressed in roots, but the underlying reason is not well understood (Stockel and Oelmüller, J. BIOL. CHEM. 279:10243 (2004); Sullivan and Gray, PLANT CELL ONLINE 11:901 (1999); Yabe et al., PLANT CELL ONLINE 16:993 (2004)). It has been inferred that plastids in dark-treated shoots and/or in roots could signal the expression of nuclear-encoded photosynthesis genes (Sullivan and Gray, PLANT CELL ONLINE 11:901 (1999)). These results suggest that plastids in maize seminal roots are able to trigger the expression of nuclear-encoded photosynthetic genes, but that those in nodal roots are not able to do so. As expected, probe sets corresponding to photosynthetic genes were less expressed in roots as compared to foliar tissues.

TABLE 6
Photosynthetic genes up-regulated in seminal roots compared to nodal roots.

| | | | Description |
|---|---|---|---|
| | | | (direct description or transfer |
| Name | GO terms | GO source | from target) |
| GRMZM2G013342 | GO:0015979 photosynthesis | IPR003685 | Photosystem I protein PsaD |
| | GO:0009538 photosystem I reaction center | | |
| GRMZM2G149428 | GO:0016020 membrane | IPR001344 | Chlorophyll A-B binding protein |
| | GO:0009765 photosynthesis, light harvesting | | |
| GRMZM2G017290 | GO:0015979 photosynthesis | IPR003666 | Photosystem I reaction centre |
| | GO:0009538 photosystem I reaction center | | protein PsaF, subunit III |
| GRMZM2G024150 | GO:0015979 photosynthesis | IPR003685 | Photosystem I protein PsaD |
| | GO:0009538 photosystem I reaction center | | |
| GRMZM2G012397 | GO:0009522 photosystem I | IPR017493 | — |
| | GO:0015979 photosynthesis | IPR016370 | — |
| | | IPR000549 | Photosystem I PsaG/PsaK protein |
| GRMZM2G016066 | GO:0003824 catalytic activity | IPR008990 | Electron transport accessory protein |
| | GO:0009536 plastid | IPR003375 | Photosystem I reaction centre |
| | GO:0015979 photosynthesis | | subunit IV/PsaE |
| | GO:0009538 photosystem I reaction center | | |
| GRMZM2G161673 | GO:0046406 magnesium protoporphyrin IX | IPR013217 | — |
| | methyltransferase activitv | | |
| | GO:0015995 chlorophyll biosynthetic process | IPR013216 | Methyltransferase type 11 |
| | GO:0050825 ice binding | IPR010940 | Magnesium-protoporphyrin IX |
| | | | methvltransferase, C-terminal |
| | GO:0042309 homoiothermy | IPR010251 | Magnesium protoporphyrin O- |
| | | | methvltransferase |
| | GO:0050826 response to freezing | IPR000104 | Antifreeze protein, type I |
| | GO:0015979 photosynthesis | | |
| GRMZM2G451224 | GO:0015979 photosynthesis | IPR004928 | Photosystem I reaction centre |
| | GO:0009538 photosystem I reaction center | | subunit VI |
| GRMZM2G377855 | GO:0009522 photosystem I | IPR017494 | — |
| | GO:0015979 photosynthesis | IPR000549 | Photosystem I PsaG/PsaK protein |
| GRMZM2G448174 | GO:0031361 integral to thylakoid membrane | IPR002325 | Cytochrome f |
| | GO:0015979 photosynthesis | | |
| | GO:0020037 heme binding | | |
| | GO:0009055 electron carrier activity | | |

One cis-acting motif was identified in the promoters of genes up-regulated in seminal roots as compared to nodal roots. This motif, referred to as a Maize Seminal Root (MSR) motif, is MSR1 (SEQ ID NO:27), which is is similar to the anaerobic response motif (AACAGGG) associated with abiotic stress (Mohanty et al., ANNALS BOTANY 96:669 (2005)). MSR1 is also similar to the E-box motif (AACAGG) implicated in the binding of class AbHLH transcription factors (Hsu et al., MOL. CELL. BIOL. 14:1256 (1994)).

Two over-represented cis-acting motifs were identified in the promoter regions of genes up-regulated in nodal roots as compared to seminal roots. These motifs, referred to as Maize Nodal Root (MNR) motifs, include MNR1 and MNR2. MNR1 (SEQ ID NO:28), is similar to the CT-rich motif known to be an enhancer of the CaMV 35S promoter (Pauli et al., J. VIROL. 78:12120 (2004)). MNR1 is also similar to a motif (CTCTCTC) over-represented in dark-induced Arabidopsis promoters that are under diurnal regulation (Janaki and Joshi, IN SILICO BIOL. 4:149 (2004)), which may be to help explain the differences in the expression of photosynthesis genes discussed above. The reverse complement of MNR1 is similar to the GAGAG elements in Drosophila that bind transcription factors containing a GAF domain, which domain is conserved in plant sensory proteins (e.g., ethylene receptors), including those involved in phytochrome B signaling (Su and Lagarias, PLANT CELL ONLINE 19:2124 (2007); van Steensel et al., PROC. NATL. ACAD. SCI. 100:2580 (2003)). MNR2 (SEQ ID NO:29) is similar to the A-box subfamily of ACGT elements (ACGTA), which have been shown to bind bZIP transcription factors (Foster et al., FASEB J. 8:192 (1994)).

### Differential Gene Expression in Juvenile and Adult Plants.

Juvenile Versus Adult Leaves:

A total of 187 probe sets, representing 96 genes, were differentially expressed between leaves of juvenile plants and leaves of adult plants; genes were up-regulated in leaves from juvenile plants as compared to leaves from adult plants and genes were up-regulated in leaves from adult plants as compared to leaves from juvenile plants. The differentially expressed probe sets represented various types of genes.

Probe sets corresponding to several interesting categories of genes were up-regulated in leaves from juvenile plants as compared to leaves from adult plants. For instance, probe sets corresponding to genes associated with growth (e.g., fatty acid biosynthesis) and transferase activity were up-regulated in leaves from juvenile plants.

Although numerous probe sets were up-regulated in leaves from adult plants as compared to leaves from juvenile plants, GO annotation categories were assigned because only 24 genes could be confidently identified as corresponding to the up-regulated probe sets. Interestingly, five of the probe sets that were up-regulated in leaf 8 at the V5 stage (adult plant) as compared to leaves from juvenile plants were not expressed in leaf 8 at any other stage of development nor were they expressed in any other vegetative tissue at any stage of development. One of these probe sets corresponded to a gene encoding an uncharacterized DUF607 superfamily protein containing a coiled-coiled domain also found in mitochondrial calcium uniporter proteins (Interpro Protein Domain: IPR006769, GRMZM2G425863). The remaining probe sets had no clear gene annotation in any plant genome. The promoters of these genes may serve as useful markers of adult vegetative development.

Two over-represented cis-acting motifs were identified in the promoter regions of genes up-regulated in juvenile leaves as compared to adult leaves. Those motifs, which we refer to as Maize Juvenile Leaf (MJL) motifs, are described in Table 7. The approximate positions of MJL1 (SEQ ID NO:30) and MJL2 (SEQ ID NO:31) in the promoter regions of the up-regulated genes (Preferential position) are shown therein.

Three over-represented cis-acting motifs were identified in the promoter regions of genes up-regulated in adult leaves as compared to juvenile leaves. Those motifs, which we refer to as Maize Adult Leaf (MAL) motifs, are described in Table 7. The approximate positions of MAL1 (SEQ ID NO:34), MAL2 (SEQ ID NO:35) and MAL3 (SEQ ID NO:36) in the promoter regions of the up-regulated genes (preferential position) are shown therein.

TABLE 7
Exemplary over-represented cis-acting motifs identified in the promoter
regions of genes corresponding to probe sets that were differentially
expressed in tissue from juvenile and adult plants

| | | Preferential | |
|---|---|---|---|
| Motif name | Consensus/Reverse | position | Up-regulated in . . . |
| MJL1 | TTCACCTTCCA/TGGAAGGTGAA | −200 −150 | juvenile leaves |
| MJL2 | TACCTTTTC/GAAAAGG | −100 −50 | juvenile leaves |
| MJR1 | TCCTTTTCCT/AGGAAAAGGA | −500 −450 | juvenile roots |
| MJR2 | TGGTTTCCCT/AGGGAAACCA | −500 −450 | juvenile roots |
| MAL1 | CATGCAACAA/TTGTTGCATG | −300 −250 | adult leaves |
| MAL2 | ACACCACG/CGTGGTGT | −100 −50 | adult leaves |
| MAL3 | CCC [symbol] /AACGGGGG | −50 +1 | adult leaves |
| MAR1 | GTTTGTTTG/CAAACAAAC | −150 −100 | adult roots |
| MAR2 | TNGAAACAAA/TTTGTTTCNA | −200 −150 | adult roots |
| MAR3 | CCACGC/GCGTGG | −100 −50 | adult roots |
| MAR4 | T [symbol] TCCC/GGGAAACGGA | −50 +1 | adult roots |
| MAR5 | CAGACTGC/GCAGTCTG | −50 +1 | adult roots |

Juvenile Versus Adult Roots:

A total of 370 probe sets, representing 208 genes, were differentially expressed between roots of leaf 4/V2 stage (juvenile) and roots at the leaf 8/V5 stage (adult; seminal and nodal roots pooled). The differentially expressed probe sets represented various types of genes. Genes that were up-regulated in both juvenile and adult stage roots included those related to tetrapyrrole and heme/iron binding. Roots require the tetrapyrrole siroheme, an essential co-factor for nitrite and sulfite reduction (Mochizuki et al., TRENDS PLANT SCI. 15:488 (2010)). Genes that were up-regulated only in juvenile stage roots were related to oxido-reductive activities, known also to be involved in nutrient uptake and assimilation (Dechorgnat et al., J. EXP. BOTANY 62:1349 (2011)). Genes only up-regulated in adult-stage roots were related to antioxidant and oxidative stress responses, known to be involved in hormonal control of root development including auxin regulation of the root apical meristem (De Tullio et al., BIOLOGIA PLANTARUM 48:161 (2004)).

Two over-represented cis-acting motifs were identified in the promoter regions of genes up-regulated in juvenile V2 roots as compared to adult roots and termed Maize Juvenile Root (MJR) motifs (see Table 7). The approximate positions of MJR1 (SEQ ID NO:32) and MJR2 (SEQ ID NO:33) in the promoter regions of the up-regulated genes (Preferential position) are shown in Table 7.

Five over-represented cis-acting motifs were identified in the promoter regions of genes up-regulated in V5 roots compared to V2 roots, and we refer to these as motifs of Maize Adult Roots (MAR1-5). These motifs are referred to as Maize Adult Root (MAR) motifs and are described in Table 7. The approximate positions of MAR1 (SEQ ID NO:37), MAR2 (SEQ ID NO:38), MAR3 (SEQ ID NO:39), MAR4 (SEQ ID NO:40) and MAR5 (SEQ ID NO:41) in the promoter regions of the up-regulated genes (Preferential position) are provided. As shown in Table 7, MAR1 and MAR2 have a similar AAAACAA core.

### Commonalities Amongst Juvenile-Specific and Adult-Specific Promoter Motifs.

To further characterize the age-related differences in gene expression in tissues taken from juvenile (V2) plants and adults (V5) plants, the over-represented cis-acting motifs identified in juvenile/adults leaves and roots were compared.

Juvenile Leaf and Juvenile Root Promoter Motifs Share Common Elements:

As shown in Table 7, the two cis-acting promoter motifs identified in leaves from juvenile plants, MJL1 (SEQ ID NO:30) and MJL2 (SEQ ID NO:31), and the two cis-acting promoter motifs identified in leaves from juvenile roots, MJR1 (SEQ ID NO:32) and MJR2 (SEQ ID NO:33), share a common pair of adjacent trinucleotides (shown in bold and italics, respectively). Furthermore, MJL2 and MJR1 share a common 7 bp sequence (underlined) wherein the adjacent trinucleotides are separated by the same nucleotide. Although this type of apparent motif sharing might be considered an artifact if probe sets corresponding to many of the same genes had been up-regulated in juvenile leaves and juvenile roots, out of the 122 promoter regions (34 from leaves and 88 from roots) used to identify over-represented cis-acting promoter motifs in juvenile plants, only 3 were shared by juvenile leaves and juvenile roots. These data suggest that juvenile-selective promoters in maize roots and shoots may be activated by a common family of transcription factors, suggesting root-shoot coordination of gene expression.

The MJL1,2 and MJR1,2 motifs (reversed: 7-10 G/A nucleotides) resembled the Motif 2 binding site of MADS-box transcription factor, FLOWERING LOCUS C (FLC) (Deng et al., PROC. NATL. ACAD. SCI. 108:6680 (2011)). FLC is a key regulator of vegetative and reproductive transitions in Arabidopsis (Id.). FLC binding site Motif 2 represented 39% of the 505 gene targets bound by FLC in Arabidopsis, and was shown to be G/A rich AGAMOUS (Id). The MJL1,2 and MJR1,2 motifs also resembled Motifl of FLC and other MADS-box factors [CArG-box, consensus 5′-CC(A/T)₄NNGG-3′] which include APETALA (AP1,3 but not AP2) and AGAMOUS (Riechmann et al., NUCLEIC ACIDS RES. 24:3134 (1996); Shiraishi et al., PLANT J. 4:385 (1993)). It has been reported that a terminal AAA followed by GG nucleotides are highly conserved in the CArG box (Deng et al., PROC. NATL. ACAD. SCI. 108:6680 (2011)); this sequence was found in MJL1, MJL2 and MJR1. The core of MJL1 (CTTCC) also exactly matched part of the MADS Agamous transcription factor binding site AG301 (Shiraishi et al., PLANT J. 4:385 (1993)). This was of interest because an APETAL1-like protein and a MADS box protein were associated with phase change in this study (Table 7, see text below).

The MJL and MJR motifs also resembled the degenerate binding site of GT transcription factors in plants called the GT element. GT elements contain one or two G nucleotides followed by four-five T or A nucleotides [5′-G-(A/G)-(T/A)-A-A-(T/A)-3′]. When reversed, MJR1 contains the 6 bp binding site (GT-lconsensus motif, GGAAAA) for GT-1-like trihelix transcription factors, which have diverse functions in plants including mediating dark/light cues and stress tolerance responses (e.g. involving salicylic acid) (Zhou, TRENDS PLANT SCI. 4:210 (1999)). The core of MJR2 matches the GT-motif binding site (TGGTTT) of AtMYB2 (Hoeren et al., GENETICS 149:479 (1998)). GT-1 and GT-2 transcription factors are related to S1F factors (see below) and have been shown to be expressed in both root and shoot (Villain et al., J. BIOL. CHEM. 271:32593 (1996)), consistent with the motif being detected in both juvenile root and shoot expressed promoters.

MJL and MJR motifs also matched additional promoter motifs from the literature. The GAAAAG core sequence common to MJL2 and MJR1 were present in a 10 bp motif (GAAAAGc/tGAA) responsible for gene expression in plant embryo suspensor tissues (Kawashima et al., PROC. NATL. ACAD. SCI. 106:3627 (2009)). The core of MJL2 and MJR1 was identical to the pyrimidine box (P-box, CCTTTT) that comprises one of the tripartite cis-acting elements required for gibberellin (GA) responses, termed the GA responsive complex (GARC) (Gubler and Jacobsen, PLANT CELL ONLINE 4:1435 (1992); Rogers et al., PLANT PHYSIOL. 105:151 (1994); Skriver et al., PROC. NATL. ACAD. SCI. 88:7266 (1991)). The reverse sequences of MJL2 and MJR1 were also identical to the cis-element (AAAAG) that binds Dof (DNA binding with one finger) transcription factors implicated in numerous processes including carbon metabolism, light- and leaf-regulated gene regulation (Yanagisawa and Sheen, PLANT CELL ONLINE 10:75 (1998)). Finally, the MJR1 core also matched the NF-AT binding site (reversed, TTTTCC) in mammals (Tsytsykova et al., J. BIOL. CHEM. 271:3763 (1996)).

These data suggest common G/A-rich promoter motifs, perhaps binding sites for FLC, help to coordinate root and leaf gene expression associated with juvenility in maize.

Adult Leaf and Adult Root Promoter Motifs Share Common Elements:

As shown in Table 7, each of the three cis-acting promoter motifs identified in leaves from adult plants, MAL 1 (SEQ ID NO:34), MAL2 (SEQ ID NO:35) and MAL3 (SEQ ID NO:36) shared at least one common sequence with at least one of the five cis-acting promoter motifs identified in leaves from adult roots, MAR1 (SEQ ID NO:37), MAR2 (SEQ ID NO:38), MAR3 (SEQ ID NO:39), MAR4 (SEQ ID NO:40) and MAR5 (SEQ ID NO:41), or the reverse complement thereof. The MAL1 motif, the reverse complement of the MAR1 motif and the MAR2 motif share a common AACAA element (shown in italics); that element is extended to comprise a common nucleotide on either end (AAACAAA, underlined) in the MAR2 motif and the reverse complement of the MAR1 motif. The MAL2 and MAR3 motifs share a common CCACG element (shown in bold italics), The MAL3 and MAR4 motifs share a common CCGTT element (shown in bold italics). Although this type of apparent motif sharing might be considered an artifact if probe sets corresponding to many of the same genes had been up-regulated in adult leaves and adults roots, out of the 147 promoter regions (24 from leaves and 123 from roots) used to identify over-represented cis-acting promoter motifs in adult plants, only 3 were shared by adult leaves and adult roots. These data suggest that related transcription factors may regulate adult vegetative phase identity in both roots and shoots in maize.

The 5 bp core of MAL1 (AACAA) matched the core of MAR1 and MAR2 (Table 7). These sequences matched the AACA-box shown to bind a subset of MYB transcription factors in plants, including MYB regulators of abiotic stress (MYBCORE, reversed CTGTTG) and seed storage protein expression (AACAA or AACAAA) (Takaiwa et al., PLANT MOL. BIOL. 30:1207 (1996); Urao et al., PLANT CELL ONLINE 5:1529 (1993)). This core motif also overlapped the sequence CAACA in MAL1. (with similar sequences in MAR1 and MAR2) which binds the N-terminus of the AP2 transcription factor domain (Kagaya et al., NUCLEIC ACIDS RES. 27:470 (1999)). This result was of interest as an AP2-like transcription factor (GLOSSY15) has been shown to regulate the juvenile-adult transition in maize leaf epidermis Evans et al., DEV. 120:1971 (1994); Moose and Sisco, GENES DEV. 10:3018 (1996)). The enlarged core of MAR1 and MAR2 (reversed, TTTGTTT) also matched the 7 bp core binding site of FOX D3 [Forkhead box D3, consensus A(a/t)T(a/g)TTTGTTT], a winged helix transcription factor family transcription factor in animals required to maintain pluripotent stem cells during embryogensis (Sutton et al., J. BIOL. CHEM. 271:23126 (1996)). MAL1 contains the internal 5 bp (TTGTT) of FOX D3. In general, TTTGTTT is associated with many developmentally regulated promoters in animals (Id.).

The 5 bp core of MAL2 (CCACG) matched the core of MAR3 (Table 7). This core is identical to the binding site for bZIP proteins that require cooperative binding, such as with NF-Y factors (e.g. UPR promoters, ERSE box) (Liu and Howell, PLANT CELL ONLINE K 22:782 (2010); Martinez and Chrispeels, PLANT CELL ONLINE 15:561 (2003); Schindler et al., PLANT CELL ONLINE 4:1309 (1992); Yoshida et al., MOL. CELL. BIOL. 21:1239 (2001)).

The 5 bp core of MAL3 (CCGTT) matched the core of MAR4 (Table 7). This sequence, when reversed (AACGG) matched the core of the MYB transcription factor binding site (MYBcore) in plants (Planchais et al., PLANT MOL. BIOL. 50:109 (2002)). In MAL3, this motif overlapped a sequence similar to the anoxia inducible cis-element CCCCCG (Gupta and Goldwasser, NUCLEIC ACIDS RES. 24:4768 (1996); Mann et al., BMC BIOTECH. K 11:74 (2011)).

Finally, the 5′ region of MAL1 (CATGC) and the 3′ region of MAR5 (ACTGC) weakly resembled one another, and contained an ATG trinucleotide core (pink, Table 2). The MAL1 sequence is identical to the pentanucleotide core of the RY-motif (g/c)CATGCxx(g/c) in plants which mediates developmental gene expression (Bobb et al., NUCLEIC ACIDS RES. 25:641 (1997); Hoffman and Donaldson, EMBO J. 4:883 (1985)). In MAR5, the CATGC core overlapped the recognition site (CAGAC) of the Smad transcription factor family in the TGFβ signalling pathway (Demler et al., EMBO J. 17:3091 (1998); Pais et al., RNA 16:489 (2010)). Intriguingly, a Smad-interacting protein (DAWDLE, containing a Forkhead Associated Domain) has recently been characterized in Arabidopsis and is involved in the biogenesis of miRNAs; this is of interest because parallel regulators (Serrate, Hasty) of miRNA processing affect juvenile to adult phase change in plants (Yu et al., PROC. NATL. ACAD. SCI. 105:10073 (2008)). Finally, MAR5 was related to an ABA responsive motif (ABRE motif III) (Shen et al., PLANT MOL. BIOL. 54:111 (2004)).

It is noteworthy that MAR1, MAR2 and MAR5 all had putative associations with Forkhead transcription factors. It may be that one or members of this family play an important regulatory role in adult roots in maize.

These results suggest that related MYB and bZIP transcription factors, and possibly AP2, Forkhead and RY motif binding proteins, regulate adult stage gene expression in maize roots and leaves. The existence of multiple binding protein matches to each of our retrieved motifs may simply be an artifact of probability, or may reflect cooperative regulation, for example different transcription factors that respond to developmental age compared to organ type.

Shared Phase Change Genes Associated with Juvenility or Adulthood:

A total of 14 probe sets, representing 12 genes, were differentially expressed in both the leaves and roots of juvenile plants as compared to the leaves and roots of adult plants. These genes may thus be critical for vegetative phase change as their expression is based on developmental phase rather than organ type. This shared list of genes included at least two genes putatively linked to wax biosynthesis (Table 8).

TABLE 8
Genes corresponding to probe sets differentially expressed in both the leaves and roots of juvenile/adult plants

| | | | Leaves | | | Roots | | |
|---|---|---|---|---|---|---|---|---|
| | Description | | Fold | Average | | Fold | Average | |
| Probe | (probe set similarity | Homology protein | change | expression | | change | expression | |
| set | to existing protein) | in maize genome | (log2) | (log2) | P-value | (log2) | (log2) | P-value |
| 1 | Apetala1-like protein | GRMZM2G147716_P01 | 7.46 | 7.86 | 2.14E−44 | 4.40 | 7.86 | 2.46E−37 |
| 2 | CER1 expressed | GRMZM2G066578_P01 | 3.71 | 7.29 | 2.35E−06 | 3.99 | 7.29 | 5.45E−15 |
| 3 | CER1 protein | GRMZM2G066578_P01 | 3.32 | 6.62 | 1.21E−05 | 3.73 | 6.62 | 6.34E−15 |
| 4 | Arabidopsis long chain | GRMZM2G101875_P02 | −2.55 | 8.20 | 8.42E−09 | −2.19 | 8.20 | 7.61E−13 |
| | acyl-CoA synthetase 1 | | | | | | | |
| 5 | Putative rice protein | GRMZM2G151567_P01 | 6.36 | 7.06 | 5.52E−31 | 5.11 | 7.06 | 1.36E−35 |
| | SHR5-receptor-like | | | | | | | |
| | kinase | | | | | | | |
| 6 | No Description | na | 2.22 | 8.56 | 9.15E−06 | 2.04 | 8.56 | 1.56E−10 |
| 7 | No Description | na | 2.57 | 5.60 | 0.000522 | 2.23 | 5.60 | 5.16E−07 |
| 8 | hypothetical protein | na | 2.48 | 7.13 | 0.000291 | 2.08 | 7.13 | 7.75E−07 |
| 9 | m28 protein, LpMADS3 | GRMZM2G147716_P01 | 5.52 | 7.78 | 6.61E−39 | 2.03 | 7.78 | 3.82E−17 |
| 10 | m28 protein, LpMADS3 | GRMZM2G147716_P01 | 6.93 | 9.87 | 3.78E−42 | 4.86 | 9.87 | 8.38E−42 |
| 11 | Glossy1-like gene, rice | GRMZM2G066578_P01 | 3.85 | 6.66 | 2.50E−06 | 3.86 | 6.66 | 2.83E−13 |
| | CER1 protien | | | | | | | |
| 12 | Putative rice protein | GRMZM2G151567_P01 | 7.07 | 5.93 | 4.69E−34 | 5.40 | 5.93 | 6.21E−37 |
| | SHR5-receptor-like | | | | | | | |
| | kinase | | | | | | | |
| 13 | Putative rice protein | GRMZM2G151567_P01 | 6.87 | 5.61 | 4.30E−31 | 5.71 | 5.61 | 4.86E−37 |
| | SHR5-receptor-like | | | | | | | |
| | kinase | | | | | | | |
| 14 | Putative rice protein | GRMZM2G151567_P01 | 6.23 | 4.96 | 9.29E−27 | 4.87 | 4.96 | 3.33E−30 |
| | SHR5-receptor-like | | | | | | | |
| | kinase | | | | | | | |

In maize, differences in epicuticular wax are used as visual markers to distinguish juvenile from adult leaves (Poethig, SCIENCE 250:923 (1990)). During the early steps of wax biosynthesis, C16 and C18 long-chain fatty acids are synthesized in plastids, and they are subsequently mobilized to the cytosol where they are activated by co-enzyme A by a long chain acyl-CoA synthetase (CER8 in Arabidopsis) (Lü et al., PLANT J. 59:553 (2009)). Later, long-chain acyl-CoAs can be modified to alkanes, perhaps by CER1 in Arabidopsis, a putative aldehyde decarbonylase (Aarts et al., PLANT CELL ONLINE 7:2115 (1995); Lü et al., PLANT J. 59:553 (2009); Sturaro et al., PLANT PHYSIOL. 138:478 (2005)). One of the differentially expressed probes matched a long chain acyl-CoA synthetase while three probes matched CER1, of which one was annotated as Glossy 1 in maize (probe set 11, Table 8). Glossy1 is an ortholog of Cer1; GLOSSY1 is active in juvenile leaves but not in adult leaves, and a mutation in Glossy1 causes the adult wax phenotype in juvenile leaves in maize (Sturaro et al., PLANT PHYSIOL. 138:478 (2005)). To our knowledge, a change in wax composition has not previously been noted during the juvenile to adult transition in maize roots. However, leaf wax biosynthesis pathway enzyme GL8 has been reported to be expressed in maize roots (Xu et al., PLANT PHYSIOL. 115:501 (1997)). These results suggest that that the juvenile to adult vegetative transition in maize involves coordinated changes in epicuticular wax gene expression in both roots and shoots, a novel finding.

Among probes corresponding to regulatory genes that were regulated by developmental phase rather than organ type were: an SHR5-receptor-like kinase; an APETALA1-like protein; and a MADS box protein orthologous to MADS3 in Lolium perenne (Table 8) (Petersen et al., J. PLANT PHYSIOL. 161:439 (2004)). APETALA (AP) and MADS-box proteins are known to play significant regulatory roles during plant development (Bommert et al., PLANT CELL PHYSIOL. 46:69 (2005)). As noted above, motifs moderately resembling the binding sites of AP/MADS-box proteins were over-represented in gene expression clusters in this study.

### Differential Gene Expression in Pre-Flowering and Post-Flowering Plants.

Microarray analysis was used to further characterize the age-related differences in gene expression in the leaves and roots of pre-flowering (Ve, V1, V2 and V5 (i.e., growing coleoptile, growing juvenile leaves, and growing adult leaf 8)) and post-flowering (R1, 10DAP, 17DAP, 24DAP and 31DAP) plants. For these comparisons, leaves/roots were pooled within each of the two stages of development.

Growing Pre-Flowering Leaves Versus Mature Post-Flowering Leaves:

The leaf results integrated changes caused by the floral transition (pre- versus post-flowering), the position of the leaf on the stalk (lower versus higher), as well as the metabolic age of the leaves (growing versus expanded). A total of 4936 probes, representing 9.1% of the probes expressed (in at least one of the 50 tissues of the complete microarray data set), were differentially expressed in expanding lower leaves during juvenile/adult stages compared to fully-expanded higher leaves during reproductive development: 2409 probes were up-regulated in growing lower leaves while 2527 probes were up-regulated in expanded, higher leaves. Genes associated with small conjugating protein ligase activity, required for ubiquitin-mediated protein degradation, were over-represented in mature leaves during reproductive stages, consistent with senescence and/or nutrient scavenging.

A subset of the differentially expressed probes (100 probes with the highest p-values, representing 47 genes) was used to search for over-represented promoter motifs. One motif, which we refer to as a motif of Maize Old Reproductive Stage Leaves (MORSL1, consensus ACCATT (SEQ ID NO:43); preferential location: −400-350, −150-100) was associated with promoters that were up-regulated in higher leaves during reproductive stages. This motif matched the core of the S1F binding site (CCATG, CCATT, CCATT) conserved in many plastid related genes (Zhou et al., J. BIOL. CHEM. 267:23515 (1992)), and exactly matched the S1F1 site (−160 bp) in the nuclear-encoded plastid ribosomal protein L21 gene (Lagrange et al., PLANT CELL ONLINE 9:1469 (1997)). The S1F1 motif appears to mediate repression of genes required for photosynthesis in non-photosynthetic tissues (Villain et al., J. BIOL. CHEM. 269:16626 (1994)), consistent with photosynthesis being repressed in older leaves compared to young growing leaves. This interpretation is also consistent with an earlier study showing that the largest class of genes up-regulated in juvenile leaves compared to leaf 9 (adult leaves) was associated with photosynthesis (Strable et al., PLANT J. 56:1045 (2008)).

In lower, pre-flowering growing leaves, GO annotations showed that genes associated with various biosynthetic activities were over-represented, including cellulose biosynthesis and iron scavenging (enterobactin biosynthesis), along with genes involved in microtubule movement. Growing tissues are dependent on membrane trafficking and cytoskeleton reorganization to permit cell growth and division (Hussey et al., ANN. REV. PLANT BIOL. 57:109 (2006); Jiirgens, ANN. REV, CELL DEV. BIOL. 20:481 (2004)). These results are consistent with gene expression directed towards leaf growth.

A subset of the differentially expressed probes (100 probes with the highest p-values, representing 65 genes) was used to search for over-represented promoter motifs associated with gene expression in lower, growing leaves at pre-flowering stages. A motif which we refer to as a motif of Maize Young Lower Leaves (MYLL1, consensus GGAACG (SEQ ID NO:42; preferential location: −50-100), was over-represented in the promoters of genes that were up-regulated in lower, growing leaves. A literature search suggests that this motif has not been identified in plants; however when reversed (CGTTCC), it exactly matches a putative cis-element over-represented in the promoters of the yeast transcription factor family YAP, a member of the bZIP family which is abundant in plants (Fernandes et al., MOL. CELL. BIOL. 17:6982 (1997); Kielbasa et al., BIOINFORMATICS 17:1019 (2001)).

Pre Flowering Roots Versus Post-Flowering Roots:

94 probes, representing 80 genes, were down-regulated in juvenile/adult roots (Ve-V5 stage, seminal and nodal roots combined) compared to roots of reproductive stage shoots. No genes with an inverse expression pattern were identified (IUT test).

An unusual feature of the up-regulated post-flowering root cluster was an over-abundance of genes linked to mitochondrial transport and ATP/GTP-dependent helicase activity. RNA helicases remodel RNA and RNA-protein complexes involved in ribosome biogenesis, mRNA splicing and export, translation and degradation (Linder and Jankowsky, NAT. REV. MOL. CELL. BIOL. 12:505 (2001)). DEAD-box RNA helicases have been implicated in helping to mediate root responses to potassium (K+) and iron (Fe) (Ricachenevsky et al., MOL. BIOL. REP. 37:3735 (2010); Xu et al., FEBS J. 278:2296 (2011)).

Two over-represented cis-acting motifs, referred to as motifs of Maize Old Reproductive Stage Roots (MORSR), were over-represented in the promoters of genes that were down-regulated in pre-flowering roots (juvenile and adult) but up-regulated in reproductive stage roots: G(g/c)GTGATT (SEQ ID NO:44) (MORSR1) and GGGCCNG (SEQ ID NO:45) (MORSR2). The reverse complement of the MORSR1 motif matched the CACCC box which binds an uncharacterized protein in the promoter of maize histone genes (Brignon and Chaubet, PLANT J.K 4:445 (1993)). In Drosophila and mammals, CACCC elements bind Kruppel-like zinc fingers involved in cell proliferation and death, differentiation and development (Pearson et al., INTL. J. BIOCHEM. CELL BIOL. 40:1996 (2008)). The core of MORSR2 was identical to the SORLIP2AT cis-element (Sequences Over-Represented in Light-Induced Promoters, GGGCC) which was computationally identified based on its high frequency in the promoters of phytochrome A-induced genes in Arabidopsis (Hudson and Quail, PLANT PHYSIOL. 133:1605 (2003)).

### Example 3

### Expression of Promoter Motifs in Transgenic Plants

A β-glucuronidase (GUS) reporter gene construct can be generated by cloning truncated promoters (i.e., minimal promoters), both wild-type and mutated variants, into a Gateway® cloning vector (Life Technologies, Grand Island, N.Y.). Using standard molecular biology techniques known in the art such as restriction enzyme digestion and ligation (See, e.g., Sambrook & Russell (2001). Molecular Cloning: A Laboratory Manual, Third Edition, Cold Spring Harbor Laboratory Press, Cold Spring Harbor, N.Y., United States of America), the truncated promoters can be constructed to comprise one or more nucleotide sequences of the invention (e.g., SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, and/or SEQ ID NO:45). These recombinant promoters comprising one or more nucleotide sequences of this invention can further be constructed to comprise one or more other full or partial cis-regulatory elements. The constructs with a promoter comprising one or more of the nucleotide sequences of SEQ ID NOs:1-45 can then be operably linked to a GUS reporter, It is noted that any suitable reporter gene or a gene of interest can be operably linked to the recombinant promoter.

The construct comprising the recombinant promoter can then be stably transformed into plant cells using standard transformation procedures, such as, for example, agrobacteria-mediated transformation or particle bombardment. The resultant transgenic seedlings can be planted and sampled before and after the developmental growth stage of interest (for example, before and after the transition from juvenile to adult growth, between different juvenile stages of development, or before and after the transition from non-flowering to flowering, and the like), and the whole plant and/or selected tissues will be subjected to a standard assay for expression of the marker gene or other gene operably associated with the recombinant promoter. The expression level and pattern of expression driven by the recombinant promoter comprising one or more nucleotide sequences of SEQ ID NOs:1-45 will be compared.

The foregoing is illustrative of the present invention, and is not to be construed as limiting thereof. The invention is defined by the following claims, with equivalents of the claims to be included therein.

All publications, patent applications, patents and other references cited herein are incorporated by reference in their entireties for the teachings relevant to the sentence and/or paragraph in which the reference is presented.

1-15. (canceled)

16. An isolated nucleic acid comprising a promoter having one or more nucleotide sequences selected from the group consisting of SEQ ID NOs: 1-6, 8-26 and 30-45, wherein the promoter confers developmental stage specific transcription when operably linked to a polynucleotide of interest.

17. An isolated nucleic acid comprising a promoter having one or more nucleotide sequences of SEQ ID NO:7, SEQ ID NO:27, SEQ ID NO: 28 and/or SEQ ID NO:29, wherein the promoter confers root specific transcription when operably linked to a polynucleotide of interest.

18. The isolated nucleic acid of claim 16, wherein the promoter is operably linked to a polynucleotide of interest.

19. The isolated nucleic acid of claim 17, wherein the promoter is operably linked to a polynucleotide of interest.

20. An expression cassette comprising the isolated nucleic acid of claim 18.

21. An expression cassette comprising the isolated nucleic acid of claim 19.

22. A vector comprising the expression cassette of claim 20.

23. A vector comprising the expression cassette of claim 21.

24. A plant cell comprising the vector of claim 22.

25. A plant cell comprising the vector of claim 23.

26. A plant comprising the plant cell of claim 24.

27. A plant comprising the plant cell of claim 25.

28. A method for directing developmental stage specific transcription of a polynucleotide of interest in a plant, the method comprising:
introducing into a plant cell a nucleic acid of claim 16, wherein the promoter is operably linked to the polynucleotide; and
regenerating a plant from said plant cell, wherein the developmental stage specific transcription is juvenile root- and/or juvenile leaf-specific transcription, adult leaf-specific transcription, adult to post flowering leaf-specific transcription, coleoptile-specific transcription, juvenile leaf-specific transcription, juvenile root-specific transcription, adult leaf-specific transcription, adult root-specific transcription, preflowering leaf-specific transcription, reproductive stage leaf-specific transcription, or reproductive stage root-specific transcription, whereby the polynucleotide is expressed in juvenile roots and/or juvenile leaves, juvenile to adult transition leaves, adult to post-flowering transition leaves, coleoptiles, juvenile leaves, juvenile roots, adult leaves, adult roots, pre-flowering leaves, reproductive stage leaves, or reproductive stage roots.

29. A method for directing root-specific transcription of a polynucleotide of interest in a plant, the method comprising:
introducing into a plant cell a nucleic acid of claim 17, wherein the promoter is operably linked to the polynucleotide; and
regenerating a plant from said plant cell, whereby the polynucleotide is expressed in roots.

30. A method of producing a plant comprising the nucleic acid of claim 16, the method comprising:
introducing into a plant cell the nucleic acid of claim 16 to produce a stably transformed plant cell; and
regenerating a stably transformed plant from the plant cell.

31. A method of producing a plant comprising the nucleic acid of claim 17, the method comprising:
introducing into a plant cell the nucleic acid of claim 17 to produce a stably transformed plant cell; and
regenerating a stably transformed plant from the plant cell.

32. A stably transformed plant produced by the method of claim 30.

33. A stably transformed plant produced by the method of claim 31.

34. A seed of the plant of claim 32.

35. A seed of the plant of claim 33.

36. A product or process product harvested from the plant of claim 32.

37. A product or process product harvested from the plant of claim 33.

38. A crop comprising a plurality of the plant of claim 32.

39. A crop comprising a plurality of the plant of claim 33.
